(19) United States
(12) Patent Application Publication
(10) Pub. No.: US 2011/0118234 A1
(43) Pub. Date: May 19, 2011

(54) Urea Compounds as Gamma Secretase Modulators

(76) Inventors: Kaustav Biswas, Calabasas, CA (US); Jian J. Chen, Camarillo, CA (US); James R. Falsey, Moorpark, CA (US); Vijay Keshav Gore, Thousand Oaks, CA (US); Qingyian Liu, Camarillo, CA (US); Vu Van Ma, Simi Valley, CA (US); Stephanie J. Mercede, Thousand Oaks, CA (US); Robert M. Rzasa, Ventura, CA (US); Christopher M. Tegley, Thousand Oaks, CA (US); Jiawang Zhu, Thousand Oaks, CA (US)

(21) Appl. No.: 12/990,999

(22) PCT Filed: May 4, 2009
(86) PCT No.: PCT/US09/42711

§ 371 Date: Jan. 24, 2011

Related U.S. Application Data
(60) Provisional application No. 61/126,480, filed on May 5, 2008.
(60) Provisional application No. 61/127,434, filed on May 13, 2008.

(51) Int. Cl. A61K 31/55; C07D 213/53; A61K 31/44; C07D 405/12; A61K 31/4433; C07D 413/12; A61K 31/5377; C07D 403/12; A61K 31/47; A61K 31/4439; A61K 31/444; A61P 25/28

(52) U.S. Cl. 514/212.07; 546/332; 514/357; 546/282.7; 514/337; 544/131; 514/235.5; 546/171; 514/314; 546/283.4; 514/336; 546/273.4; 514/338; 546/275.4; 514/341; 546/272.1; 514/340; 546/265; 514/332; 540/523

(57) ABSTRACT

The present invention provides compounds Formula (I) that are gamma secretase modulators and are therefore useful for the treatment of diseases treatable by modulation of gamma secretase such as Alzheimer's disease. Also provided are pharmaceutical compositions containing such compounds and processes for preparing such compounds.

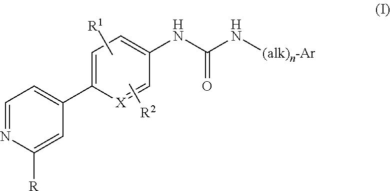

### CROSS-REFERENCE

This application claims priority to U.S. provisional application Nos. 61/126,480, filed May 5, 2008 and 61/127,434, filed May 13, 2008, the disclosures of which are incorporated herein by reference in their entirety.

### FIELD OF THE INVENTION

The present invention provides compounds that are gamma secretase modulators and are therefore useful for the treatment of diseases treatable by modulation of gamma secretase such as Alzheimer's disease. Also provided are pharmaceutical compositions containing such compounds and processes for preparing such compounds.

### BACKGROUND

Alzheimer's disease (AD) is the most common cause of dementia, resulting in loss of memory, cognition, reasoning, judgement, and orientation. AD is characterized by the presence of extracellular amyloid plaques, intracellular neurofibrillary tangles, in addition to loss of synapses and neurons in the brain. The main constituent of amyloid plaques is β-amyloid (Aβ) a 4 kDa peptide.

Accumulation of Aβ is thought to be an early and critical step in the pathogenesis of Alzheimer's Disease (AD). Aβ elicits a cascade of toxic and inflammatory events that ultimately lead to neuronal death and cognitive impairment. The Aβ peptide results from proteolysis of Amyloid Precursor Protein (APP). The APP protein is a transmembrane protein consisting of a large extracellular domain and a short cytoplasmic tail. Aβ sequence encompasses parts of the extracellular and transmembrane domains of APP.

APP can be processed via either of two routes, a non-amyloidogenic and an amyloidogenic pathway. Most of the APP is processed through the non-amyloidogenic pathway, whereby the protease α-secretase cleaves APP within the Aβ domain to release a large soluble N-terminal fragment (sAPPα) and a non-amyloidogenic C-terminal fragment (C83). This fragment is further processed by γ-secretase to produce a 22-24 residue peptide (p3).

In the amyloidogenic pathway, APP is cleaved by β-secretase (BACE1), generating a shorter N-terminal domain (sAPPβ) and an amyloidogenic C-terminal (C99). This C99 fragment is subsequently cleaved by γ-secretase. The γ-secretase is a protease formed by a complex of proteins: Presenilin-1 (PS-1), Nicastrin, PEN-2, and APH-1. Proteolysis of APP intermediates by γ-secretase yields Aβ peptides of varying length (Aβ37, Aβ38, Aβ39, Aβ40, Aβ42). Of these peptides, Aβ42 is the least soluble, most aggregating species and the principal component of toxic oligomers and amyloid plaques in AD brain. All known mutations causing early onset Familial AD either increase total Aβ formation or increase the ratio of Aβ42 to Aβ40. Therefore agents that can block the formation of Aβ42 should be useful for the treatment of AD.

One proposed treatment involves modulation of γ-secretase activity to selectively reduce the production of Aβ42 while increase the production of the shorter chain isoforms (such as Aβ37, 38, and 39). These isoforms are believed to be less prones to self-aggregate and are more easily cleared from the brain and or less toxic. Several classes of compounds are proposed as γ-secretase modulators (referred hereon as GSM), see, Imbimbo B. P, et al., J Pharmacol Exp Ther. 2007 December; 323(3):822-30; WO2007054739; WO2006043064; and WO2007124351.

The present invention provides a new class of compounds that selectively reduce the production of Aβ42 peptide by modulation of γ-secretase and hence are useful in the treatment of Alzheimer's disease.

### SUMMARY

In one aspect, provided herein are compounds of Formula (I):

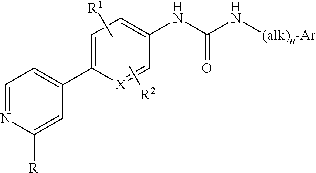

where:

X is —CH— or —N—;

n is 0 or 1;

alk is a straight or branched alkyl of 1 to 6 carbon atoms where one, two, or three hydrogen atoms of the alkyl chain are replaced by alkoxy, hydroxyl or halo;

R is alkyl;

R¹ and R² are independently hydrogen, alkyl, alkoxy, hydroxy, or halo;

Ar is:

(i) aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or
(ii) a group of formula (a):

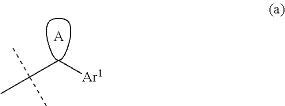

ring A is cycloalkyl optionally substituted with halo, hydroxyl, alkoxy, oxo, or haloalkyl; or monocyclic heterocyclyl wherein if the heterocyclyl ring contains a nitrogen ring atom, the nitrogen atom is optionally substituted with alkyl, or acyl, acyloxycarbonyl;

Ar¹ is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or

a pharmaceutically acceptable salt thereof provided the compound of Formula (I) is not:

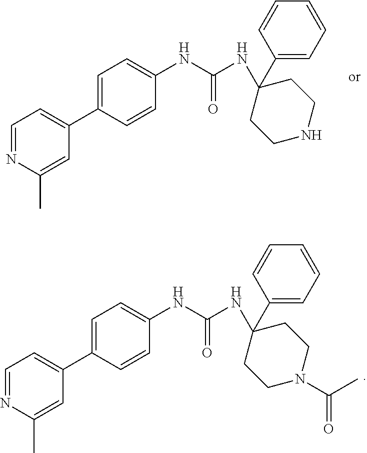

In a second aspect, provided is a pharmaceutical composition comprising a compound of Formula (I), a pharmaceutically acceptable salt thereof or a mixture of a compound of Formula (I) and a pharmaceutically acceptable salt thereof; and a pharmaceutically acceptable excipient.

In a third aspect, this invention is directed to a method of treating Alzheimer's disease by inhibition of γ-secretase in a patient which method comprises administering to the patient a pharmaceutical composition comprising a compound of Formula (I) a pharmaceutically acceptable salt thereof, or a mixture of a compound of Formula (I) and a pharmaceutically acceptable salt thereof and a pharmaceutically acceptable excipient.

In a fourth aspect, this invention is directed to an intermediate of formula:

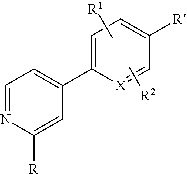

where:

X, R, R¹ and R² are as defined for Formula (I) above; and

R′ is NH₂, NHPG, —CON₃, or —N═C═O where PG is a suitable amino protecting group; or a salt thereof.

In a fourth aspect, this invention is directed to use of a compound of Formula (I) or a salt thereof as a medicament.

In a fifth aspect, this invention is directed to use of a compound of Formula (I) or a salt thereof in the preparation of a medicament for use in the treatment of Alzheimer's disease.

### DETAILED DESCRIPTION

### Definitions

Unless otherwise stated, the following terms used in the specification and claims are defined for the purposes of this application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means a —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” or “alkoxyalkoxy” means a —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, each as defined above, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means a —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —CONH₂, methylaminocarbonyl, 2-dimethylaminocarbonyl, and the like.

“Aminosulfonyl” means a —SO₂NRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetyl, propionyl, benzoyl, pyridinylcarbonyl, and the like. When R is alkyl, the radical is also referred to herein as alkylcarbonyl.

“Acylamino” means a —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined herein, e.g., acetylamino, propionylamino, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above.

“Aryloxy” means a —OR radical where R is aryl as defined above, e.g., phenoxy, naphthyloxy.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkylalkyl” means a -(alkylene)-R radical where R is cycloalkyl as defined above; e.g., cyclopropylmethyl, cyclobutylmethyl, cyclopentylethyl, or cyclohexylmethyl, and the like.

“Cycloalkoxy” means a —OR radical where R is cycloalkyl as defined above, e.g., cyclopropoxy, cyclobutoxy, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., dimethylamino, phenylmethylamino, and the like.

“Fused cycloalkyl” means cycloalkyl ring as defined above that is fused to one or two aryl or heteroaryl ring as defined herein e.g., tetrahydronaphthyl, 5,6,7,8-tetrahydroquinolinyl, and the like.

“Halo” means fluoro, chloro, bromo, or iodo, preferably fluoro or chloro.

“Haloalkyl” means alkyl radical as defined above, which is substituted with one or more halogen atoms, preferably one to five halogen atoms, preferably fluorine or chlorine, including those substituted with different halogens, e.g., —CH₂Cl, —CF₃, —CHF₂, —CH₂CF₃, —CF₂CF₃, —CF(CH₃)₃, and the like. When the alkyl is substituted with only fluoro, it is referred to in this application as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to in this application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means a —OR radical where R is hydroxyalkyl as defined above.

“Heterocyclyl” means a saturated or unsaturated monovalent monocyclic group of 5 to 8 ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this application as “bicyclic heterocyclyl” ring and when it is not fused to aryl or heteroaryl, it is referred as “monocyclic heterocyclyl”. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heterocyclylalkyl” means a -(alkylene)-R radical where R is heterocyclyl ring as defined above e.g., tetraydrofuranylmethyl, piperazinylmethyl, morpholinylethyl, and the like.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Heteroaralkyl” means a -(alkylene)-R radical where R is heteroaryl as defined above.

“Heteraryloxy” means a —OR radical where R is heteroaryl as defined above, e.g., pyridinyloxy, thiophenyloxy, and the like.

“Monosubstituted amino” means a —NHR radical where R is alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., methylamino, 2-phenylamino, hydroxyethylamino, and the like.

“Modulation of γ-secretase activity” as used herein means the production of Aβ42 produced by γ-secretase is reduced in the presence of the compounds of the Invention.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfonyl” means a —SO₂R radical where R is alkyl, haloalkyl, aryl, aralkyl, heteroaryl, heteroaralkyl, heterocyclyl, or heterocyclylalkyl, each as defined herein, e.g., methylsulfonyl, phenylsulfonyl, benzylsulfonyl, pyridinylsulfonyl, and the like.

The phrase in the definition of group Ar in the claims and in the specification of this application “ . . . wherein each of the aforementioned ring is optionally substituted with R^{a}, R^{b}, or R^{c} independently selected from . . . ” and similar phrases used for others groups in the claims and in the specification with respect to the compound of Formula (I) means that the rings can be mono-, di-, or trisubstituted unless indicated otherwise.

“Treating” or “treatment” of a disease includes:

preventing the disease, i.e. causing the clinical symptoms of the disease not to develop in a mammal that may be exposed to or predisposed to the disease but does not yet experience or display symptoms of the disease;

inhibiting the disease, i.e., arresting or reducing the development of the disease or its clinical symptoms; or

relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

Representative compounds of the Invention are shown in Tables 1 and 2 below:

Compounds of Formula (I) where Ar is n, alk, and Ar are shown below are.

TABLE 1

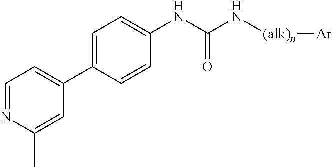

| Cpd # | Stereo | n | Alk | Ar |
|---|---|---|---|---|
| 1 | (R) | 1 | —CH(CH₃)— | naphth-1-yl |
| 2 | — | 1 | —(CH₂)₂— | 2,7-dimethylindol-3-yl |
| 3 | — | 0 | 0 | 1,2,3,4-tetrahydronaphth-1-yl |
| 4 | — | 1 | —(CH₂)— | naphth-1-yl |
| 5 | — | 1 | —(CH₂)₂— | 5-chloro-2-methylindol-3-yl |
| 6 | (R) | 0 | 0 | 2-(pyridin-2-yl)-1,2,3,4- |
| | | | | tetrahydronaphth-1-yl |
| 7 | — | 1 | —(CH₂)₂— | indol-3-yl |
| 8 | — | 1 | —(CH₂)₂— | 5-bromo-2-methylindol-3-yl |
| 9 | — | 1 | —C(CH₃)₂— | phenyl |
| 10 | — | 0 | 0 | 6-chloro-2,2-dimethylbenzopyran-4-yl |
| 11 | — | 1 | —(CH₂)₂— | 2-methyl-7-(2-propyl)indol-3-yl |
| 12 | (R) | 1 | —CH(CH₃)— | 2-chlorophenyl |
| 13 | — | 1 | —(CH₂)₂— | 1,2-dimethylindol-3-yl |
| 14 | — | 0 | 0 | 6-bromo-2,2-dimethylbenzopyran-4-yl |
| 12 | — | 1 | —CH₂— | 3-trifluoromethylphenyl |
| 13 | — | 1 | —(CH₂)₂— | 4-fluorophenyl |
| 14 | — | 0 | 0 | (S)-6-ethyl-2-methyl-2- |
| | | | | methoxymethylbenzopyran-4-yl |
| 15 | (S) | 1 | —CH(CH₃)— | 2-chlorophenyl |
| 16 | (S) | 0 | 0 | 1,2,3,4-tetrahydronaph-1-yl |
| 17 | — | 0 | 0 | 9H-fluoroen-9-yl |
| 18 | — | 1 | —(CH₂)₂— | 2-methylindol-3-yl |
| 19 | — | 1 | —(CH₂)₂— | 2,4-dichlorophenyl |
| 20 | — | 0 | 0 | 6,8-dichloro-2,2-dimethylbenzopyran-4-yl |
| 21 | — | 1 | —CH₂— | 2-chloro-6-trifluoromethylphenyl |
| 22 | (R) | 1 | —CH(CH₃)— | phenyl |
| 23 | (R) | 1 | —CH(CH₃)— | cyclohexyl |
| 24 | — | 0 | 0 | naphth-1-yl |
| 25 | — | 0 | 0 | (S)-2-tert-butyl-7,7-dimethyl-5,6,7,8- |
| | | | | tetrahydroquinazolin-5-yl |
| 26 | — | 1 | —C(CH₃)₂— | 4-fluorophenyl |
| 27 | — | 1 | —CH₂CH(CH₃)— | phenyl |
| 28 | — | 1 | —CH₂— | 3-fluoro-6-trifluoromethylphenyl |
| 29 | — | 1 | —(CH₂)₃— | phenyl |
| 30 | — | 1 | —(CH₂)₃— | 2-fluorophenyl |
| 31 | — | 1 | —CH₂— | 2-difluoromethoxyphenyl |
| 32 | — | 0 | 0 | 3,5-ditrifluoromethylphenyl |
| 33 | — | 0 | 0 | 2,3-dihydro-1H-inden-1-yl |
| 34 | — | 1 | —CH₂— | biphen-3-yl |
| 35 | — | 1 | —(CH₂)₂— | 3-methoxyphenyl |
| 36 | — | 1 | —C(CH₃)₂— | 6-chloropyridin-2-yl |
| 37 | — | 1 | —(CH₂)₂— | 2-pyridin-3-ylindol-3-yl |
| 38 | — | 1 | —CH₂— | 3-methylphenyl |
| 39 | (R) | 0 | 0 | 6-(hydroxymethyl)-1,2,3,4- |
| | | | | tetrahydronaphth-1-yl |
| 40 | (R) | 0 | 0 | 1,2,3,4-tetrahydronaphth-2-yl |
| 41 | — | 0 | 0 | 2-(2-pyridin-2-ylethyl)phenyl |
| 42 | (S) | 0 | 0 | 6-ethyl-3,4-dihydro-2H-chromen-4-yl |
| 43 | — | 1 | —CH(CH₃)— | 2,3-difluoropyridin-4-yl |
| 44 | — | 0 | 0 | 2,6-dichloropyridin-4-yl |
| 45 | — | 0 | 0 | 2,3,4,5-tetrahydro-1H-benzo[b]azepin-5-yl |
| 46 | — | 1 | —CH₂— | benzothiophen-3-yl |
| 47 | — | 0 | 0 | 4-[1,2,4]triazol-1-ylphenyl |
| 48 | — | 0 | 0 | 6-bromopyridin-3-yl |
| 49 | — | 0 | 0 | 2,3-dihydrobenzo[b][1,4]dioxin-7-yl |
| 50 | — | 1 | —(CH₂)₂— | thiophen-2-yl |
| 51 | — | 0 | 0 | 4-pyrazol-1-ylphenyl |
| 52 | — | 0 | 0 | 4-oxazol-5-ylphenyl |
| 53 | — | 1 | —CH₂— | 3-morpholin-4-ylphenyl |
| 54 | (R) | 1 | —CH₂— | 6-(4-methylpyridin-1- |
| | | | | ylmethyl)1,2,3,4-tetrahydronaphth-1-yl |
| 55 | — | 0 | 0 | biphen-4-yl |
| 56 | — | 1 | —CH₂— | 1-hydroxycyclohexyl |
| 57 | (R) | 1 | —CH(CH₃)— | naphth-2-yl |
| 58 | (S) | 1 | —CH(CH₃)— | naphth-2-yl |
| 59 | — | 1 | —CH(CH₃)CH₂— | 4-fluorophenyl |
| 60 | — | 1 | —CH(CH₃)— | 2-fluorophenyl |
| 61 | — | 1 | —CH₂— | naphth-2-yl |
| 62 | — | 0 | 0 | 4(R)-3,3-dimethyl-3,4-dihydro-2H- |
| | | | | chromen-4-yl |
| 63 | — | 1 | —CH(CH₃)— | 3-fluorophenyl |
| 64 | — | 1 | —(CH₂)₂— | 4-bromophenyl |
| 65 | — | 1 | —CH(CH₃)— | 2-methoxyphenyl |
| 66 | — | 1 | —CH(n-C₃H₇)— | phenyl |
| 67 | — | 1 | —CH(CH₃)— | 4-bromophenyl |
| 68 | — | 1 | —CH(CH₃)— | 3-methoxyphenyl |
| 69 | — | 0 | 0 | (1R,2S)-2-phenylcyclopropyl |
| 70 | (R) | 1 | —CH(C₂H₅)— | phenyl |
| 71 | (S) | 1 | —CH(C₂H₅)— | phenyl |
| 72 | — | 1 | —(CH₂)₂— | 4-chlorophenyl |
| 73 | — | 1 | —(CH₂)₂— | 4-cyanophenyl |
| 74 | — | 0 | — | 3-morpholin-4-ylphenyl |
| 75 | | 1 | —CH(CH₃)— | 4-methylphenyl |
| 76 | | 1 | —CH(CH₃)— | 4-chlorophenyl |
| 77 | | 0 | — | quinolin-5-yl |
| 78 | | 1 | —CH(CH₃)— | 3-chlorophenyl |
| 79 | | 1 | —CH(CH₃)— | 3-bromophenyl |
| 80 | | 1 | —CH(CH₃)— | 2-bromophenyl |
| 81 | | 1 | —CH₂— | quinolin-4-yl |
| 82 | (R) | 1 | —CH(CH₃)— | quinolin-2-yl |
| 83 | (S) | 1 | —CH(CH₃)— | quinolin-2-yl |
| 84 | (R) | 1 | —CH(CH₃)— | quinolin-4-yl |
| 85 | (S) | 1 | —CH(CH₃)— | quinolin-4-yl |
| 86 | | 1 | —CH₂— | quinolin-2-yl |
| 87 | (R) | 1 | —CH(CH₃)— | 2-fluorophenyl |
| 88 | (S) | 1 | —CH(CH₃)— | 2-fluorophenyl |
| 89 | | 0 | — | 5-bromo-2-fluorophenyl |
| 90 | | 1 | —CH(CH₃)— | furan-2-yl |
| 91 | | 1 | —CH(CH₃)— | benzofuran-2-yl |
| 92 | | 1 | —CH(CH₃)— | 1-difluoromethylbenzimidazol-2-yl |
| 93 | | 1 | —CH(CH₃)— | 1-ethyl3,5-dimethylpyrazol-4-yl |
| 94 | | 0 | — | tetrahydropyran-3-yl |
| 95 | | 1 | CH₂ | 3-methylisoxazol-5-yl |
| 96 | | 0 | — | tetrahydrofuran-3-yl |
| 97 | | 1 | —CH(CF₃)— | phenyl |
| 98 | | 1 | —CH(CH₃)— | 2-chlorophenyl |
| 99 | | 1 | —CH(CH₃)— | 2-methylphenyl |
| 100 | | 1 | —CH(CH₃)— | 5-fluoropyridin-2-yl |
| 111 | | 1 | —CH(CH₃)— | 5-chloropyridin-2-yl |
| 112 | | 1 | —CH(CH₃)— | 6-methoxypyridin-3-yl |
| 113 | | 1 | —CH(CH₃)— | 3-difluoromethoxyphenyl |
| 114 | | 1 | —CH(CH₃)— | isoquinolin-3-yl |
| 115 | | 1 | —CH(CH₃)— | quinolin-7-yl |
| 116 | | 1 | —CH(CH₂F)— | phenyl |
| 117 | | 1 | —CH(CH₂OCH₃)— | phenyl |
| 118 | | 1 | —CH[CHF(CH₃)]₂— | phenyl |
| 119 | | 1 | —CH(CH₃)— | 2,4-dichlorophenyl |
| 120 | | 1 | —CH(CH₃)— | 4-methoxyphenyl |
| 121 | | 1 | —CH(CH₃)— | 3-methyl-2-oxo-2,3,4,5-tetrahydro- |
| | | | | 1H-benzo[d]azepin-1-yl |
| 122 | | 0 | — | 2-bromophenyl |
| 123 | | 0 | — | 3′-fluorobipheny-3-yl |
| 124 | | 0 | — | 4′-methoxybiphen-2-yl |
| 125 | | 0 | — | 3-furan-2-ylphenyl |
| 126 | | 0 | — | 3-n-butoxyphenyl |
| 127 | | 0 | — | 3′-methoxybiphen-3-yl |
| 128 | | 0 | — | 2-propylthiophenyl |
| 129 | | 0 | — | 3-thiophen-3-ylphenyl |
| 130 | | 0 | — | 3-pyridin-4-ylphenyl |
| 131 | | 0 | — | 2-fluoro-5-cyanophenyl |
| 132 | | 0 | — | 5-cyano-2-methylphenyl |
| 133 | | 0 | — | 2-thiophen-2-ylphenyl |
| 134 | | 0 | — | 3-pyridin-3-ylphenyl |
| 135 | | 0 | — | 5-chloro-2-fluorophenyl |
| 136 | | 0 | — | 3-ethoxyphenyl |
| 137 | | 0 | — | 3-thiophen-2-ylphenyl |
| 138 | | 0 | — | 2-methyl-5-trifluoromethylphenyl |
| 139 | | 0 | — | 3-n-propoxyphenyl |
| 140 | | 0 | — | 2-n-propoxyphenyl |
| 141 | | 0 | — | 3-tert-butylphenyl |
| 142 | | 0 | — | 2-pyridin-3-ylphenyl |
| 143 | | 0 | — | 5-bromo-2-methylphenyl |
| 144 | | 0 | — | 2-n-propylphenyl |
| 145 | | 0 | — | 5-chloro-2-methylphenyl |
| 146 | | 0 | — | 3-ethoxyphenyl |
| 147 | | 0 | — | 2-fluoro-5-trifluoromethylphenyl |
| 148 | | 0 | — | 2-ethylphenyl |
| 149 | | 0 | — | 3-methylthiophenyl |
| 150 | | 0 | — | 2,5-difluorophenyl |
| 151 | | 0 | — | 4′-fluorobiphen-3-yl |
| 152 | | 0 | — | 5-fluoro-2-methoxyphenyl |
| 153 | | 0 | — | 2-cyanophenyl |
| 154 | | 0 | — | 3-(2-methylthiazol-4-yl)phenyl |
| 155 | | 0 | — | biphen-2-yl |
| 156 | | 0 | — | 2-cyanophenyl |
| 157 | | 0 | — | 2-benzoylphenyl |
| 158 | | 0 | — | 2-trifluoromethylphenyl |
| 159 | | 0 | — | 5-fluoro-2-methylphenyl |
| 160 | | 0 | — | 2-chloro-5-methoxyphenyl |
| 161 | | 0 | — | 2-chloro-5-methylphenyl |
| 162 | | 0 | — | 2-phenoxyphenyl |
| 163 | | 0 | — | 3-cyanophenyl |
| 164 | | 0 | — | 2-acetylphenyl |
| 165 | | 0 | — | 2-methoxybiphen-5-yl |
| 166 | | 0 | — | 2-cyano-5-methylphenyl |
| 167 | | 0 | — | 3-isopropoxyphenyl |
| 168 | | 0 | — | 3-benzoylphenyl |
| 169 | | 0 | — | 2-chloro-5-cyanophenyl |
| 170 | | 0 | — | 4′-cyanobiphen-3-yl |
| 171 | | 0 | — | 2-fluoro-5-methylphenyl |
| 172 | | 0 | — | 3-phenoxyphenyl |
| 173 | | 0 | — | 2-methoxy-5-methylphenyl |
| 174 | | 0 | — | 2-chloro-5-trifluoromethylphenyl |
| 175 | | 0 | — | 2,5-dichlorophenyl |
| 176 | | 0 | — | 2-fluorophenyl |
| 177 | | 0 | — | 3-methoxyphenyl |
| 178 | | 0 | — | 3-ethylphenyl |
| 179 | | 0 | — | 2-methylphenyl |
| 180 | | 0 | — | 2-methylthiophenyl |
| 181 | | 0 | — | 2-methoxy-5-trifluoromethylphenyl |
| 182 | | 0 | — | 5-chloro-2-cyanophenyl |
| 183 | | 0 | — | 2-ethoxyphenyl |
| 184 | | 0 | — | 2-isopropylphenyl |
| 185 | | 0 | — | 3-tert-butylphenyl |
| 186 | | 0 | — | 2-isobutylphenyl |
| 187 | | 0 | — | 2-methoxyphenyl |
| 188 | | 0 | — | 2-trifluoromethylphenyl |
| 189 | | 0 | — | 3-methylphenyl |
| 190 | | 0 | — | 3-fluorophenyl |
| 191 | | 0 | — | 5-isopropyl-2-methylphenyl |
| 192 | | 0 | — | R-3,3-dimethyltetrahydropyran-4-yl |
| 193 | | 0 | — | cyclohexyl |
| 194 | | 0 | — | cyclopentyl |
| 195 | | 0 | — | phenyl |
| 196 | | 1 | —CH₂— | furan-2-yl |
| 197 | | 0 | — | 2-methyl-5-trifluoromethylfuran-4-yl |
| 198 | | 0 | — | 5-methyl-1-phenylpyrazol-4-yl |
| 199 | | 0 | — | 5-methyl-3-phenylisoxazol-4-yl |
| 200 | | 0 | — | 3-methyl-5-phenylisoxazol-4-yl |
| 201 | | 0 | — | 2,5-dimethyl-1-phenylpyrazol-4-yl |
| 202 | | 0 | — | 2,6-dichlorophenyl |
| 203 | | 0 | — | 3-chloro-4-methoxyphenyl |
| 204 | | 0 | — | 5-chloro-2-methoxyphenyl |
| 205 | | 0 | — | 5-chloro-2,4-dimethoxyphenyl |
| 206 | | 0 | — | 3,5-difluorophenyl |
| 207 | | 1 | —CH[CH₂CH(CH₃)₂]— | 2-bromophenyl |
| 208 | | 1 | —CH(CH₃)— | 4-pyridin-3-ylphenyl |
| 209 | | 1 | —CH(CH₃)— | 3-cyanophenyl |
| 210 | | 1 | —CH(CH₃)— | 4-cyanophenyl |
| 211 | | 1 | —CH(CH₃)— | 3-methylphenyl |
| 212 | | 1 | —CH(CH₃)— | quinolin-6-yl |
| 213 | | 1 | —CH(CH₃)— | 3-methoxyquinolin-6-yl |
| 214 | | 1 | —CH[CH₂CH(CH₃)₂]— | phenyl |
| 215 | | 1 | —CH(CHF₂)— | phenyl |
| 216 | | 1 | —CH(CH₃)— | isoquinolin-6-yl |
| 217 | | 1 | —CH(CH₃)— | 3,5-ditrifluoromethylphenyl |
| 218 | | 0 | — | 4-ethoxy-5-isopropyl-2-methylphenyl |
| 219 | | 0 | — | 4-phenylthiazol-2-yl |
| 220 | | 1 | —CH₂C(CH₃)₂— | phenyl |
| 221 | | 1 | —C(CH₃)₂CH₂— | phenyl |
| 222 | | 1 | —C(CH₃)₂— | 2-fluorophenyl |
| 223 | | 0 | 1 | 1-methylcyclohexyl |
| 224 | | 1 | —C(CH₃)(CH₂CH₃)CH₂— | phenyl |
| 225 | | 1 | —C(CH₃)₂— | 3-fluorophenyl |
| 226 | | 1 | —C(CH₃)₂— | thiophen-3-yl |
| 227 | | 1 | —C(CH₃)₂— | benzofuran-5-yl |
| 228 | | 1 | —C(CH₃)₂— | thiophen-5-yl |
| 229 | | 1 | —C(CH₃)₂— | 5-bromothiophen-2-yl |
| 230 | | 1 | —C(CH₃)₂— | 4-bromothiophen-2-yl |

Table 2 shows representative compounds of Formula (I) where Ar is a ring of formula (a) are shown below.

TABLE 2

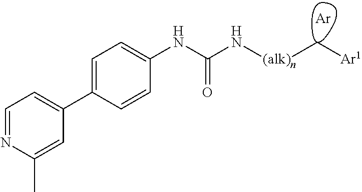

| Cpd # | n | Alk | A | Ar¹ |
|---|---|---|---|---|
| 1 | 0 | 0 | cyclobutyl | phenyl |
| 2 | 0 | 0 | cyclohexyl | phenyl |
| 3 | 0 | 0 | 1-(tert-butoxy- | phenyl |
| | | | carbonyl)piperidin-4-yl | |
| 4 | 0 | 0 | cyclopentyl | phenyl |
| 5 | 0 | 0 | cyclopropyl | 4-F-phenyl |
| 6 | 0 | 0 | tetrahydropyran-4-yl | phenyl |
| 7 | 0 | 0 | tetrahydropyran-4-yl | 2-F-phenyl |
| 8 | 0 | 0 | 1-methoxycarbonyl- | phenyl |
| | | | piperidin-4-yl | |
| 9 | 1 | —CH₂— | cyclopentyl | phenyl |
| 10 | 1 | —CH₂— | cyclobutyl | phenyl |
| 11 | 1 | —CH₂— | cyclopropyl | phenyl |
| 12 | 1 | —CH₂— | tetrahydropyran-4-yl | phenyl |
| 13 | 1 | —CH₂— | cyclopropyl | phenyl |
| 14 | 0 | — | tetrahydrofurn-3-yl | 4-chlorophenyl |
| 15 | 0 | — | cyclobutyl | 4-bromophenyl |
| 16 | 0 | — | cyclohexyl | 3-fluorophenyl |
| 17 | 0 | — | cyclohexyl | 2-fluorophenyl |
| 18 | 1 | —CH₂— | cyclopentyl | 6-chloropyridin-2-yl |
| 19 | 1 | —CH₂— | cyclopentyl | pyridine-2-yl |
| 20 | 0 | — | cyclobutyl | 3 -bromophenyl |
| 21 | 1 | —CH₂— | cyclopentyl | 2-chloropyridin-4-yl |
| 22 | 1 | —CH₂— | cyclopentyl | pyridin-4-yl |
| 23 | 1 | —CH₂— | cyclopentyl | 2-cyanopyridin-4-yl |
| 24 | 0 | — | cyclobutyl | 4-cyanophenyl |
| 25 | 0 | — | cyclopentyl | 6-chloropyridin-2-yl |
| 26 | 0 | — | cyclopentyl | pyridin-2-yl |
| 27 | 0 | — | cyclopentyl | 2-chloropyridin-4-yl |
| 28 | 0 | — | cyclopentyl | pyridin-4-yl |
| 29 | 0 | — | cyclopentyl | 5-bromopyridin-4-yl |
| 30 | 0 | — | cyclopentyl | 6-fluoropyridin-2-yl |
| 31 | 0 | — | cyclobutyl | 6-chloropyridin-2-yl |
| 32 | 0 | — | cyclopentyl | pyridin-3-yl |
| 33 | 0 | — | cyclopentyl | 6-methoxypyridin-2-yl |
| 34 | 0 | — | 4-oxocyclohexyl | phenyl |
| 35 | 0 | — | 4-hydroxycyclohexyl | phenyl |
| 36 | 0 | — | 4,4-difluorocyclohexyl | phenyl |
| 37 | 1 | —CH₂— | cyclobutyl | 2-methoxyphenyl |

### EMBODIMENTS

A. In one embodiment, the compound of Formula (I) is:

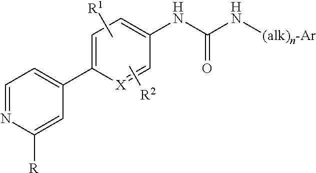

where:

X is —CH— or —N—;

n is 0 or 1;

alk is a straight or branched alkyl of 1 to 6 carbon atoms where one, two, or three hydrogen atoms of the alkyl chain are replaced by alkoxy, hydroxyl or halo;

R is alkyl;

R¹ and R² are independently hydrogen, alkyl, alkoxy, hydroxy, or halo;

Ar is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or

a pharmaceutically acceptable salt thereof.

I. Within A above, in one embodiment, the compound of Formula (I) is represented by the structure:

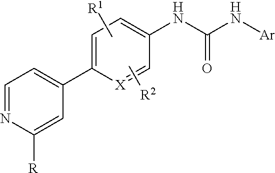

(a) Within embodiment (I), one group of compounds is that wherein X is —N—.
(b) Within embodiment (I), another group of compounds is that wherein X is —CH—.
(c) Within embodiment (I), yet another group of compounds is that wherein R¹ and R² are hydrogen.

Within group (c), one group of compounds is that wherein X is —CH—.

Within group (c), another group of compounds is that wherein X is —N—.

Within groups (a), (b) and (c) and groups contained therein, in one group of compounds R is methyl or ethyl, more preferably methyl.

(i) Within groups (a) through (c) and groups contained therein, one group of compounds is that wherein Ar is aryl optionally substituted as described above. Within this group, one group of compounds is that wherein Ar is phenyl or naphthyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl.

Within this group, another group of compounds is that wherein Ar is naphth-1-yl, phenyl, 2-chlorophenyl, 3-trifluoromethylphenyl, 4-fluorophenyl, 2-chlorophenyl, 2,4-dichlorophenyl, 2-chloro-6-trifluoromethylphenyl, 3-fluoro-6-trifluoromethylphenyl, 2-fluorophenyl, 2-difluoromethoxyphenyl, 3,5-ditrifluoromethylphenyl, biphen-3-yl, 3-methoxyphenyl, 3-methylphenyl, 2-(2-pyridin-2-ylethyl)phenyl, 4-[1,2,4]triazol-1-ylphenyl, 4-pyrazol-1-ylphenyl, 4-oxazol-5-ylphenyl, 3-morpholin-4-ylphenyl, or biphen-4-yl.

(ii) Within groups (a) through (c) and groups contained therein, another group of compounds is that wherein Ar is heteroaryl optionally substituted as described above. Within this group, one group of compounds is that wherein Ar is pyridyl, thienyl, furanyl, indolyl, benzothiophenyl, quinolinyl, isoquinolinyl, pyrimidinyl, pyrrolyl, pyrazolyl, pyridazinyl, pyrazinyl, benzimidazolyl, or benzoxazolyl optionally substituted as described above. Within this group, one group of compounds is that wherein Ar is heteroaryl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl. Within this group, another group of compounds is that wherein Ar is 2,7-dimethylindol-3-yl, 5-chloro-2-methylindol-3-yl, indol-3-yl, 5-bromo-2-methylindol-3-yl, 2-methyl-7-(2-propyl)indol-3-yl, 1,2-dimethylindol-3-yl, 2-methylindol-3-yl, 6-chloropyridin-2-yl, 2-pyridin-3-ylindol-3-yl, 2,3-difluoropyridin-4-yl, 2,6-dichloropyridin-4-yl, benzothiophen-3-yl, 6-bromopyridin-3-yl, or thiophen-2-yl.
(iii) Within groups (a) through (c) and groups contained therein, another group of compounds is that wherein Ar is cycloalkyl or heterocyclyl optionally substituted as described above. Within this group, one group of compounds is that wherein Ar cycloalkyl or heterocyclyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl. Within this group, another group of compounds is that wherein Ar is 1,2,3,4-tetrahydronaphth-1-yl, 2-(pyridin-2-yl)-1,2,3,4-tetrahydronaphth-1-yl, 6-chloro-2,2-dimethylbenzopyran-4-yl, 6-bromo-2,2-dimethylbenzopyran-4-yl, (S)-6-ethyl-2-methyl-2-methoxymethylbenzopyran-4-yl, 6,8-dichloro-2,2-dimethylbenzopyran-4-yl, cyclohexyl, (S)-2-tert-butyl-7,7-dimethyl-5,6,7,8-tetrahydroquinazolin-5-yl, 2,3-dihydro-1H-inden-1-yl, 6-(hydroxymethyl)-1,2,3,4-tetrahydronaphth-1-yl, 1,2,3,4-tetrahydronaphth-2-yl, 6-ethyl-3,4-dihydro-2H-chromen-4-yl, 2,3,4,5-tetrahydro-1H-benzo[b]azepin-5-yl, 2,3-dihydrobenzo[b][1,4]dioxin-7-yl, 6-(4-methylpyridin-1-ylmethyl)1,2,3,4-tetrahydronaphth-1-yl, or 1-hydroxycyclohexyl.
(iv) Within groups (a) through (c) and groups contained therein, another group of compounds is that wherein Ar is fused cycloalkyl optionally substituted as described above. Within this group, one group of compounds is that wherein Ar fused cycloalkyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl.
(v) Within the groups (a) through (c) and groups contained therein, another group of compounds is that wherein Ar is aryl, heteroaryl, cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, or acyl and R^{b} and R^{c} are independently selected from alkyl, halo, haloalkyl, aryl, heteroaryl, heterocyclyl, or aryloxy; where the aromatic or alicyclic ring in R^{a}, R^{b} and R^{c} is optionally substituted with R^{d}, R^{e} or R^{f} which are independently selected from alkyl, halo, cyano, or alkoxy; preferably Ar is 3-morpholin-4-ylphenyl; 4-methylphenyl; 4-chlorophenyl; 2-chlorophenyl; 3-chlorophenyl; 3-bromophenyl; 2-bromophenyl; 2-fluorophenyl; 3-fluorophenyl; 2,4-dichlorophenyl; 4-methoxyphenyl; 3-difluoromethoxyphenyl; 5-bromo-2-fluorophenyl; phenyl; 2-methylphenyl; 3-n-butoxyphenyl; 2-fluoro-5-cyanophenyl; 5-cyano-2-methylphenyl; 5-chloro-2-fluorophenyl; 3-ethoxyphenyl; 2-methyl-5-trifluoromethylphenyl; 3-n-propoxyphenyl; 2-n-propoxyphenyl; 3-tert-butylphenyl; 2-pyridin-3-ylphenyl; 5-bromo-2-methylphenyl; 2-n-propylphenyl; 5-chloro-2-methylphenyl; 3-ethoxyphenyl; 2-fluoro-5-trifluoromethylphenyl; 2-ethylphenyl; 3-methylthiophenyl; 2,5-difluorophenyl; 5-fluoro-2-methoxyphenyl; 2-cyanophenyl; 2-benzoylphenyl; 2-trifluoromethylphenyl; 5-fluoro-2-methylphenyl; 2-chloro-5-methoxyphenyl; 2-chloro-5-methylphenyl; 2-phenoxyphenyl; 3-cyanophenyl; 2-acetylphenyl; 2-cyano-5-methylphenyl; 3-isopropoxyphenyl; 3-benzoylphenyl; 2-chloro-5-cyanophenyl; 3-phenoxyphenyl; 2-methoxy-5-methylphenyl; 2-chloro-5-trifluoromethylphenyl; 2,5-dichlorophenyl; 3-methoxyphenyl; 3-ethylphenyl; 2-methylphenyl; 2-methylthiophenyl; 2-methoxy-5-trifluoromethylphenyl; 5-chloro-2-cyanophenyl; 2-ethoxyphenyl; 2-isopropylphenyl; 2-isobutylphenyl; 2-methoxyphenyl; 2-trifluoromethylphenyl; 3-methylphenyl; 5-isopropyl-2-methylphenyl; 2,6-dichlorophenyl; 3-chloro-4-methoxyphenyl; 5-chloro-2-methoxyphenyl; 5-chloro-2,4-dimethoxyphenyl; 3,5-difluorophenyl; 4-pyridin-3-ylphenyl; 4-cyanophenyl; quinolin-5-yl; quinolin-4-yl; quinolin-2-yl; furan-2-yl; benzofuran-2-yl; 1-difluoromethylbenzimidazol-2-yl; 3,5-dimethylpyrazol-4-yl; tetrahydropyran-3-yl; 3-methylisoxazol-5-yl; tetrahydrofuran-3-yl; 5-fluoropyridin-2-yl; 5-chloropyridin-2-yl; 6-methoxypyridin-3-yl; isoquinolin-3-yl; quinolin-7-yl; 3′-fluorobipheny-3-yl; 4′-methoxybiphen-2-yl; 3-furan-2-ylphenyl; 3′-methoxybiphen-3-yl; 2-propylthiophenyl; 3-thiophen-3-ylphenyl; 3-pyridin-4-ylphenyl; 2-thiophen-2-ylphenyl; 3-pyridin-3-ylphenyl; 3-thiophen-2-ylphenyl; 4′-fluorobiphen-3-yl; 3-(2-methylthiazol-4-yl)phenyl; biphen-2-yl; 2-methoxybiphen-5-yl; 4′-cyanobiphen-3-yl; R-3,3-dimethyltetrahydropyran-4-yl; cyclohexyl; cyclopentyl; phenyl; furan-2-yl; 2-methyl-5-trifluoromethylfuran-4-yl; 5-methyl-1-phenylpyrazol-4-yl; 5-methyl-3-phenylisoxazol-4-yl; 3-methyl-5-phenylisoxazol-4-yl; 2,5-dimethyl-1-phenylpyrazol-4-yl; quinolin-6-yl; 3-methoxyquinolin-6-yl; isoquinolin-6-yl; 3,5-ditrifluoromethylphenyl; 4-ethoxy-5-isopropyl-2-methylphenyl; 4-phenylthiazol-2-yl; 1-methylcyclohexyl; thiophen-3-yl; benzofuran-5-yl; thiophen-5-yl; 5-bromothiophen-2-yl; or 4-bromothiophen-2-yl.
II. Within A above, in another embodiment, the compound of Formula (I) is represented by the structure:

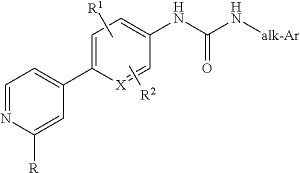

III. Within A above, in yet another embodiment, the compound of Formula (I) is represented by the structure:

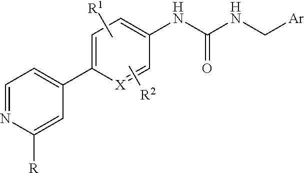

Within this group, in one group of compounds n is 1 and alk is —CH₂—, —(CH₂)₂—, —(CHCH₃)—, or —C(CH₃)₂—

IV. Within A above, in yet another embodiment, the compound of Formula (I) is represented by the structure:

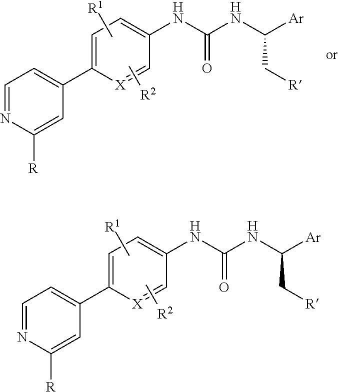

where R′ is methyl, ethyl, n-propyl, n-butyl, or isobutyl.
(a) Within embodiments (II), (III), and (IV), one group of compounds is that wherein X is —N—.
(b) Within embodiments (II), (III), and (IV), another group of compounds is that wherein X is —CH—.
(c) Within embodiments (II), (III), and (IV), yet another group of compounds is that wherein R¹ and R² are hydrogen.

Within group (c), one group of compounds is that wherein X is —CH—.

Within group (c), another group of compounds is that wherein X is —N—.

Within group (c) and groups contained therein, in one group of compounds R is methyl.

(i) Within groups (II), (III), and (IV), and groups contained therein, one group of compounds is that wherein Ar is aryl optionally substituted as described above. Within this group, one group of compounds is that wherein Ar is phenyl or naphthyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl.

Within this group, another group of compounds is that wherein Ar is naphth-1-yl, phenyl, 2-chlorophenyl, 3-trifluoromethylphenyl, 4-fluorophenyl, 2-chlorophenyl, 2,4-dichlorophenyl, 2-chloro-6-trifluoromethylphenyl, 3-fluoro-6-trifluoromethylphenyl, 2-fluorophenyl, 2-difluoromethoxyphenyl, 3,5-ditrifluoromethylphenyl, biphen-3-yl, 3-methoxyphenyl, 3-methylphenyl, 2-(2-pyridin-2-ylethyl)phenyl, 4-[1,2,4]triazol-1-ylphenyl, 4-pyrazol-1-ylphenyl, 4-oxazol-5-ylphenyl, 3-morpholin-4-ylphenyl, or biphen-4-yl.

(ii) Within groups (II), (III), and (IV), and groups contained therein, another group of compounds is that wherein Ar is heteroaryl optionally substituted as described above. Within this group, one group of compounds is that wherein Ar is pyridyl, thienyl, furanyl, indolyl, benzothiophenyl, quinolinyl, isoquinolinyl, pyrimidinyl, pyrrolyl, pyrazolyl, pyridazinyl, pyrazinyl, benzimidazolyl, or benzoxazolyl optionally substituted as described above. Within this group, one group of compounds is that wherein Ar is heteroaryl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl. Within this group, another group of compounds is that wherein Ar is 2,7-dimethylindol-3-yl, 5-chloro-2-methylindol-3-yl, indol-3-yl, 5-bromo-2-methylindol-3-yl, 2-methyl-7-(2-propyl)indol-3-yl, 1,2-dimethylindol-3-yl, 2-methylindol-3-yl, 6-chloropyridin-2-yl, 2-pyridin-3-ylindol-3-yl, 2,3-difluoropyridin-4-yl, 2,6-dichloropyridin-4-yl, benzothiophen-3-yl, 6-bromopyridin-3-yl, or thiophen-2-yl.
(iii) Within groups (II), (III), and (IV), and groups contained therein, yet another group of compounds is that wherein Ar is cycloalkyl or heterocyclyl optionally substituted as described above. Within this group, one group of compounds is that wherein Ar cycloalkyl or heterocyclyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl. Within this group, another group of compounds is that wherein Ar is 1,2,3,4-tetrahydronaphth-1-yl, 2-(pyridin-2-yl)-1,2,3,4-tetrahydronaphth-1-yl, 6-chloro-2,2-dimethylbenzopyran-4-yl, 6-bromo-2,2-dimethylbenzopyran-4-yl, (S)-6-ethyl-2-methyl-2-methoxymethylbenzopyran-4-yl, 6,8-dichloro-2,2-dimethylbenzopyran-4-yl, cyclohexyl, (S)-2-tert-butyl-7,7-dimethyl-5,6,7,8-tetrahydroquinazolin-5-yl, 2,3-dihydro-1H-inden-1-yl, 6-(hydroxymethyl)-1,2,3,4-tetrahydronaphth-1-yl, 1,2,3,4-tetrahydronaphth-2-yl, 6-ethyl-3,4-dihydro-2H-chromen-4-yl, 2,3,4,5-tetrahydro-1H-benzo[b]azepin-5-yl, 2,3-dihydrobenzo[b][1,4]dioxin-7-yl, 6-(4-methylpyridin-1-ylmethyl)1,2,3,4-tetrahydronaphth-1-yl, or 1-hydroxycyclohexyl.
(iv) Within groups (II), (III), and (IV), and groups contained therein, yet another group of compounds is that wherein Ar is fused cycloalkyl optionally substituted as described above. Within this group, one group of compounds is that wherein Ar fused cycloalkyl optionally substituted with R^{a} and R^{b} where R^{a} is halo, alkyl, haloalkyl, or alkoxy and R^{b} is halo, alkyl, haloalkyl, alkoxy, haloalkoxy, aryl, heteroaryl, heterocyclyl, monosubstituted amino, disubstituted amino, cyano, acyl, or aralkyl.
(v) Within groups (II), (III) and (IV), and groups contained therein, yet another group of compounds is that wherein Ar is aryl, heteroaryl, cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, or acyl and R^{b} and R^{c} are independently selected from alkyl, halo, haloalkyl, aryl, heteroaryl, heterocyclyl, or aryloxy; where the aromatic or alicyclic ring in R^{a}, R^{b} and R^{c} is optionally substituted with R^{d}, R^{e} or R^{f} which are independently selected from alkyl, halo, cyano, or alkoxy; preferably Ar is 3-morpholin-4-ylphenyl; 4-methylphenyl; 4-chlorophenyl; 2-chlorophenyl; 3-chlorophenyl; 3-bromophenyl; 2-bromophenyl; 2-fluorophenyl; 3-fluorophenyl; 2,4-dichlorophenyl; 4-methoxyphenyl; 3-difluoromethoxyphenyl; 5-bromo-2-fluorophenyl; phenyl; 2-methylphenyl; 3-n-butoxyphenyl; 2-fluoro-5-cyanophenyl; 5-cyano-2-methylphenyl; 5-chloro-2-fluorophenyl; 3-ethoxyphenyl; 2-methyl-5-trifluoromethylphenyl; 3-n-propoxyphenyl; 2-n-propoxyphenyl; 3-tert-butylphenyl; 2-pyridin-3-ylphenyl; 5-bromo-2-methylphenyl; 2-n-propylphenyl; 5-chloro-2-methylphenyl; 3-ethoxyphenyl; 2-fluoro-5-trifluoromethylphenyl; 2-ethylphenyl; 3-methylthiophenyl; 2,5-difluorophenyl; 5-fluoro-2-methoxyphenyl; 2-cyanophenyl; 2-benzoylphenyl; 2-trifluoromethylphenyl; 5-fluoro-2-methylphenyl; 2-chloro-5-methoxyphenyl; 2-chloro-5-methylphenyl; 2-phenoxyphenyl; 3-cyanophenyl; 2-acetylphenyl; 2-cyano-5-methylphenyl; 3-isopropoxyphenyl; 3-benzoylphenyl; 2-chloro-5-cyanophenyl; 3-phenoxyphenyl; 2-methoxy-5-methylphenyl; 2-chloro-5-trifluoromethylphenyl; 2,5-dichlorophenyl; 3-methoxyphenyl; 3-ethylphenyl; 2-methylphenyl; 2-methylthiophenyl; 2-methoxy-5-trifluoromethylphenyl; 5-chloro-2-cyanophenyl; 2-ethoxyphenyl; 2-isopropylphenyl; 2-isobutylphenyl; 2-methoxyphenyl; 2-trifluoromethylphenyl; 3-methylphenyl; 5-isopropyl-2-methylphenyl; 2,6-dichlorophenyl; 3-chloro-4-methoxyphenyl; 5-chloro-2-methoxyphenyl; 5-chloro-2,4-dimethoxyphenyl; 3,5-difluorophenyl; 4-pyridin-3-ylphenyl; 4-cyanophenyl; quinolin-5-yl; quinolin-4-yl; quinolin-2-yl; furan-2-yl; benzofuran-2-yl; 1-difluoromethylbenzimidazol-2-yl; 3,5-dimethylpyrazol-4-yl; tetrahydropyran-3-yl; 3-methylisoxazol-5-yl; tetrahydrofuran-3-yl; 5-fluoropyridin-2-yl; 5-chloropyridin-2-yl; 6-methoxypyridin-3-yl; isoquinolin-3-yl; quinolin-7-yl; 3′-fluorobipheny-3-yl; 4′-methoxybiphen-2-yl; 3-furan-2-ylphenyl; 3′-methoxybiphen-3-yl; 2-propylthiophenyl; 3-thiophen-3-ylphenyl; 3-pyridin-4-ylphenyl; 2-thiophen-2-ylphenyl; 3-pyridin-3-ylphenyl; 3-thiophen-2-ylphenyl; 4′-fluorobiphen-3-yl; 3-(2-methylthiazol-4-yl)phenyl; biphen-2-yl; 2-methoxybiphen-5-yl; 4′-cyanobiphen-3-yl; R-3,3-dimethyltetrahydropyran-4-yl; cyclohexyl; cyclopentyl; phenyl; furan-2-yl; 2-methyl-5-trifluoromethylfuran-4-yl; 5-methyl-1-phenylpyrazol-4-yl; 5-methyl-3-phenylisoxazol-4-yl; 3-methyl-5-phenylisoxazol-4-yl; 2,5-dimethyl-1-phenylpyrazol-4-yl; quinolin-6-yl; 3-methoxyquinolin-6-yl; isoquinolin-6-yl; 3,5-ditrifluoromethylphenyl; 4-ethoxy-5-isopropyl-2-methylphenyl; 4-phenylthiazol-2-yl; 1-methylcyclohexyl; thiophen-3-yl; benzofuran-5-yl; thiophen-5-yl; 5-bromothiophen-2-yl; or 4-bromothiophen-2-yl.
B. In another embodiment the compounds of Formula (I) are represented by the structure:

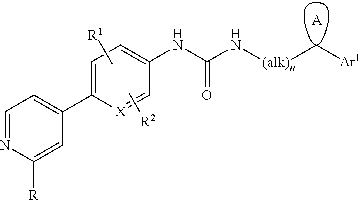

where:

X is —CH— or —N—;

n is 0 or 1;

alk is a straight or branched alkyl of 1 to 6 carbon atoms;

R is alkyl;

R¹ and R² are independently hydrogen, alkyl, alkoxy, hydroxy, or halo; ring A is cycloalkyl optionally substituted with halo, hydroxyl, alkoxy, oxo, or haloalkyl; or monocyclic heterocyclyl wherein if the heterocyclyl ring contains a nitrogen ring atom, the nitrogen atom is optionally substituted with alkyl, or acyl, acyloxycarbonyl;

Ar¹ is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino

V. Within B, in one embodiment, the compound of Formula (I) is represented by the structure (IIa):

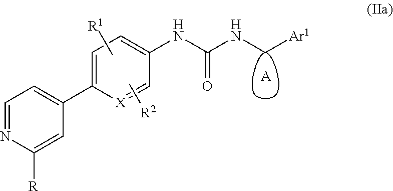

VI. Within B, in another embodiment, the compound of Formula (I) is represented by the structure (IIb).

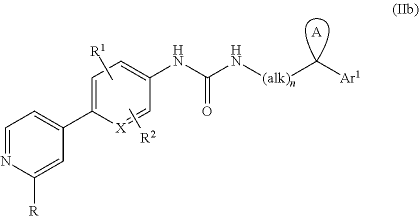

where n is 1. Within this embodiment, in one group of compounds, n is 1 and alk is —CH₂—. Within this embodiment, in another group of compounds, n is 1 and alk is —CH₂—, —(CH₂)₂—, —(CHCH₃)— or —C(CH₃)₂—;
(a′) Within embodiments (V) and (VI) and groups contained therein, one group of compounds is that wherein X is —N—.
(b′) Within embodiments (V) and (VI) and groups contained therein, another group of compounds is that wherein X is —CH—.
(c′) Within embodiments (V) and (VI) and groups contained therein, yet another group of compounds is that wherein R¹ and R² are hydrogen.

Within group (c′), one group of compounds is that wherein X is —CH—.

Within group (c′), another group of compounds is that wherein X is —N—.

(d′) Within embodiments (V), (VI), (a′), (b′), (c′) and groups contained therein; one group of compounds is that wherein R³ is methyl or ethyl. Within this group (d), in one group of compounds R is methyl.
(i) Within the embodiments (V), (VI), (a′), (b′), (c′), and (d′); and groups contained therein, one group of compounds is that wherein A is cycloalkyl. Within this group (i), in one group of compounds A is cyclopropyl, cyclobutyl, cyclopentyl, or cyclohexyl. Within this group (i), in one group of compounds A is cyclopropyl or cyclopentyl.
(ii) Within the embodiments (V), (VI), (a′), (b′), (c′), and (d′); and groups contained therein, one group of compounds is that wherein A is monocyclic heterocyclyl as defined above. Within this group (ii), in one group of compounds, A is piperidin-1-yl or tetrahydropyran-4-yl wherein the nitrogen atom of the piperidin-4-yl ring is optionally substituted with alkoxycarbonyl.
(A) Within the embodiments (V), (VI), (a′), (b′), (c′), and (d′), (i) and (ii); and groups contained therein, in one group of compounds, Ar¹ is aryl optionally substituted as described above.

Within this group, one group of compounds is that wherein Ar¹ is phenyl optionally substituted with R^{a} which is halo, alkyl, haloalkyl, or alkoxy and/or R^{b} which is halo, alkyl, haloalkyl, alkoxy, cycloalkyl, aryl, aryloxy, amino, monosubstituted amino, disubstituted amino, cyano, acyl, or aralkyl.

Within this group, another group of compounds is that wherein Ar¹ is phenyl, 2-fluorophenyl, 3-fluorophenyl, 4-chlorophenyl, 4-methoxyphenyl, 4-cyanophenyl, 3-morpholinylphenyl, 4-morphorlinylphenyl or 4-fluorophenyl.

(B) Within the embodiments (V), (VI), (a′), (b′), (c′), and (d′), (i) and (ii); and groups contained therein, in one group of compounds Ar¹ is heteroaryl optionally substituted as described above. Within this group, another group of compounds is that wherein Ar¹ is 2-chloro-6-pyridyl, or 2-chloro-6-pyrazinyl.

Within this group, one group of compounds is that wherein Ar¹ is heteroaryl optionally substituted with R^{a} which is halo, alkyl, haloalkyl, or alkoxy and/or R^{b} which is selected from halo, alkyl, haloalkyl, alkoxy, cycloalkyl, aryl, aryloxy, amino, monosubstituted amino, disubstituted amino, cyano, acyl, or aralkyl.

(C) Within the embodiments (V), (VI), (a′), (b′), (c′), and (d′), (i) and (ii); and groups contained therein, in one group of compounds Ar¹ is cycloalkyl optionally substituted as described above.
(D) Within the embodiments (V), (VI), (a′), (b′), (c′), and (d′), (i) and (ii); and groups contained therein, in one group of compounds Ar¹ is fused cycloalkyl, or heterocyclyl optionally substituted as described above.
(E) Within the embodiments (V), (VI), (a′), (b′), (c′), and (d′), (i) and (ii); and groups contained therein, in one group of compounds Ar¹ is phenyl or heteroaryl each ring optionally substituted with R^{a} which is halo, cyano, or alkoxy; preferably Ar¹ is phenyl, 4-fluorophenyl, 2-fluorophenyl, 4-chlorophenyl, 4-bromophenyl, 3-fluorophenyl, 6-chloropyridin-2-yl, pyridine-2-yl, 3-bromophenyl, 2-chloropyridin-4-yl, pyridine-4-yl, 2-cyanopyridin-4-yl, 4-cyanophenyl, 2-chloropyridin-4-yl, 5-bromopyridin-4-yl, 6-fluoropyridin-2-yl, pyridin-3-yl, 6-methoxypyridin-2-yl, or 2-methoxyphenyl.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of formula (I) where Ar is as defined in the Summary of the Invention can be prepared as described in Scheme A below.

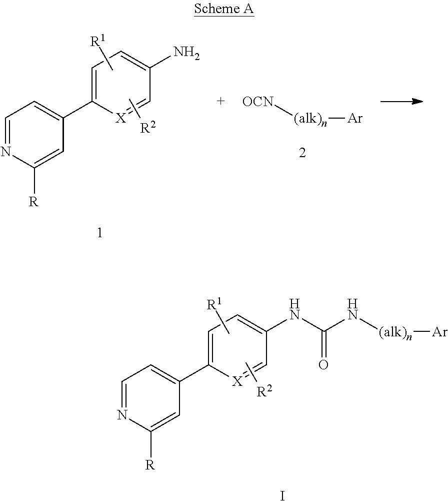

Compounds of formula (I) are synthesized by coupling of an amine of formula 1 with an isocyanate of formula 2 optionally in the presence of an organic base such as Hunig's base, pyridine, and the like and in an aprotic solvents such as THF, toluene, and the like.

Amines of formula 1 are either commercially available or can be synthesized by coupling an aryl halide of formula 3 or 6 with a boronic acid compound of formula 4 or 5 respectively, under Suzuki coupling reaction conditions as shown below:

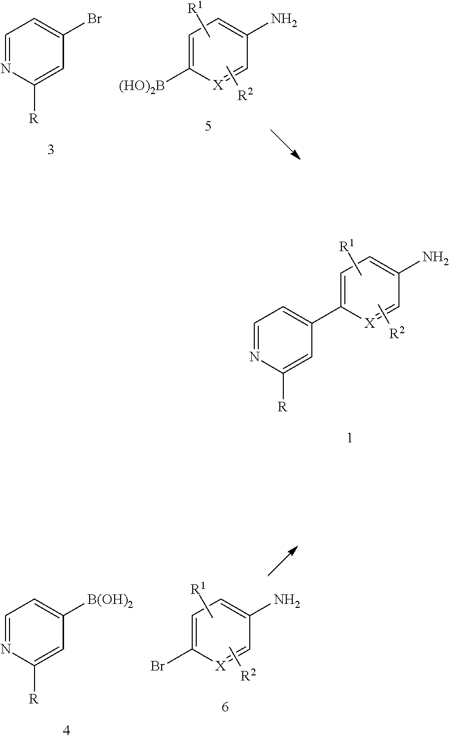

Compounds of formula 3-6 are either commercially available or prepared using conditions well known to one skilled in the art of organic synthesis.

Alternatively, compounds of Formula (I) where Ar is aryl, heteroaryl, cycloalkyl, fused cycloalkyl, or heterocyclyl can be prepared as described in Scheme B below.

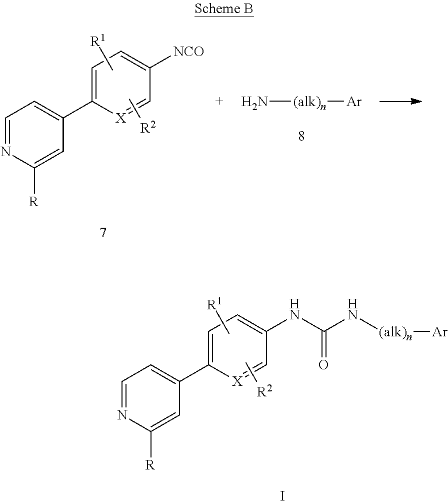

Compounds of Formula (I) can be prepared by coupling an isocyanate compound of formula 7 with an amine of formula 8 under conditions described in Scheme A above. The amine of formula 8 is either commercially available or prepared using conditions well known to one skilled in the art of organic synthesis (for example, naphthalen-1-ylmethanamine, naphthalen-2-ylmethanamine, (S)-1-(naphthalen-1-yl)ethanamines, (R)-1-(naphthalen-1-yl)ethanamines, 2-(4-bromophenyl)ethanamines, 1-phenyl-cyclopropylamine, (1-phenylcyclopentyl)methylamine, (1-phenylcyclohexyl)methylamine, (4-phenyl-tetrahydropyran-4-yl)methylamine, (1-phenylcyclopropyl)methylamine, and (1-phenylcyclobutyl)-methylamine are commercially available). The isocyanate of formula 7 can be prepared from the corresponding acid of formula 9, by first preparing an azido carbonyl compound of formula 10 by treating the acid with ethylchloroformate in the presence of an organic base such as triethyl amine, and the like and subsequent treatment with aqueous sodium azide. Heating 10 in a suitable organic solvent such as toluene, dioxane, acetonitrile, and the like, at 100-150° C., from about 1 h to 30 h provides the isocyanate derivative.

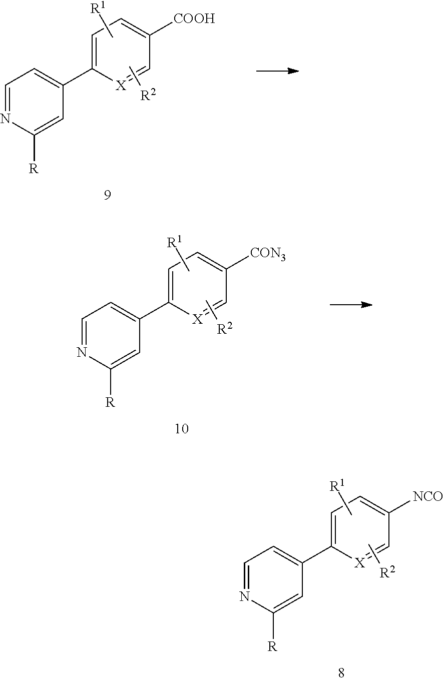

Acids of formula 9 can be prepared by Suzuki coupling of a bromide of formula 11 with a boronic acid of formula 12 where R′ is alkyl, followed by acid hydrolysis of the ester group in the resulting compound 13.

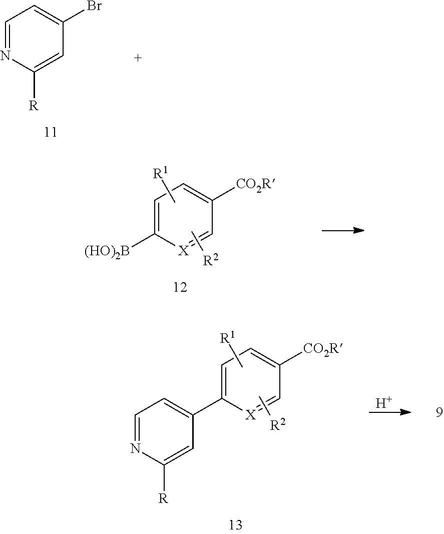

### Utility

The compounds of the invention are γ-secretase modulators and hence are useful in the treatment of Alzheimer's disease.

### Testing

The γ-secretase modulatory activity of the compounds of the present invention can be tested using the in vitro and in vivo assays described in working Example 1 below.

### Administration and Pharmaceutical Composition

In general, the compounds of this invention will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day.

For oral administration, the compositions are preferably provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this invention, i.e., the active ingredient, will depend upon numerous factors such as the severity of the disease to be treated, the age and relative health of the subject, the potency of the compound utilized, the route and form of administration, and other factors.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

### Examples

The following preparations of compounds of Formula (I) and intermediates (References) are given to enable those skilled in the art to more clearly understand and to practice the present invention. They should not be considered as limiting the scope of the invention, but merely as being illustrative and representative thereof.

### Reference A

### Synthesis of 4-(2-methylpyridin-4-yl)benzenamine

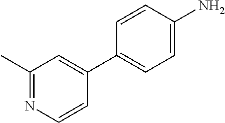

To a solution of 4-(4,4,5,5-tetramethyl-1,3,2-dioxaborolan-2-yl)benzenamine (11.7 g, 53.5 mmol), 4-bromo-2-methylpyridine (9.20 g, 53.5 mmol), Na₂CO₃ (2.83 g, 26.7 mmol) in 50 mL MeCN/50 mL H2O, was added Pd(PPh3)4 (1.85 g, 1.60 mmol), and the reaction mixture was refluxed at 130° C. for 15 h. The solution was cooled, and the solids were collected by filtration, and the residue was concentrated and extracted with ethyl acetate. Ethyl acetate solution was evaporated and the residue was combined with the solids obtained from filtration. The crude materials were subjected to silica gel chromatography with 25% EtOAc in CH₂Cl₂ gave 4-(2-methylpyridin-4-yl)benzenamine.

### Reference 2

### Synthesis of 3-methoxy-4-(2-methylpyridin-4-yl)benzenamine

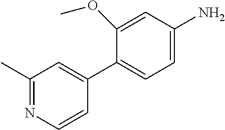

### Step 1

4-Chloro-2-picoline (3 g, 0.0235 mol) was dissolved in 1,4-dioxane (72 mL). Bis pinacolato diborane (7.76 g, 0.03 mol) was added to the reaction mixture. The reaction mixture was degassed with nitrogen for 45 mins. Potassium acetate (3.45 g, 0.035 mol), tricyclohexylphosphine (0.660 g, 2.3 mmol) and Pd(dba)₂ (0.676 g, 1.1 mmol) were added. The mixture was heated at 90° C. for 3 h. The crude reaction mixture was cooled and filtered through Celite bed and washed with ethyl acetate. The filtrate was concentrated and used directly for next reaction without purification.

### Step 2

3-Methoxy-4-bromo nitrobenzene (4.9 g, 21 mmol) was dissolved in 1,2-dimethoxyethane (60 mLl). 2-Methyl-4-(4,4,5,5-tetramethyl-[1,3,2]dioxaborolan-2-yl)-pyridine (5.15 g, 23.5 mmol) was added. Sodium carbonate (5.15 g, 48.5 mmol) was dissolved in minimum amount of water and was added drop wise to the reaction mixture. The reaction mixture was degassed with nitrogen for 45 min. Pd(PPh₃)₄ (0.732 g, 0.6 mmol) was added under nitrogen. The reaction mixture was then heated overnight at 90° C. After completion of reaction, the reaction mixture was cooled and extracted with ethyl acetate. Purification was done by column chromatography using silica gel (100-200 mesh) and 0-2% DCM, MeOH as eluent to give 4-(2-methoxy-4-nitrophenyl)-2-methylpyridine.

### Step 3

To a stirred solution of 4-(2-methoxy-4-nitrophenyl)-2-methylpyridine (15.8 g, 64.7 mmol) in 200 mL of ethanol was degassed with nitrogen and added Pd/C (6.89 g, 6.47 mmol). The reaction mixture was hydrogenated using hydrogen balloon at 25° C. The reaction mixture was filtered through celite, concentrated, and purified by crystallization using dichloromethane and pentane to give the title compound.

### Reference 3

### Synthesis of 4-nitrophenyl 4-(2-methylpyridin-4-yl)phenylcarbamate

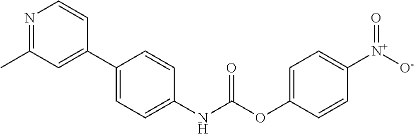

To a solution of 4-nitrophenyl carbonochloridate (4.38 g, 21.7 mmol), 1.2 eq of the polystyrene bound Hunig base, 3.6 mmol/G, Agonaut, 2.3 g) in 2:1 EtOAc/CH₂Cl₂, was added 4-(2-methylpyridin-4-yl)benzenamine (2.00 g, 10.9 mmol) in portions, and the reaction mixture was stirred at RT for 48 h. The impurities were removed by filtration, and the solids (containing product and the resin) was suspended (the product was dissolved) in 2:1 MeOH/CH₂Cl₂. The product solution (soluble in MeOH/CH₂Cl₂, not soluble in EtOAc) was filtered through medium glass-fitted funnel and concentrated to give 4-nitrophenyl 4-(2-methylpyridin-4-yl)phenylcarbamate.

### Reference 4

### Synthesis of azido(4-(2-methylpyridin-4-yl)phenyl)methanone

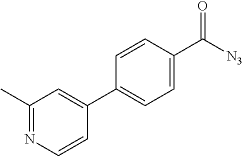

### Step 1

A solution of 4-bromo-2-methylpyridine (7.7 g, 45 mmol), 4-(methoxycarbonyl)-phenylboronic acid (10.5 g, 58 mmol) in DME (100 mL) were added Cs₂CO₃ (22 g) and 0.5 g POPd (dihydrogen dichlorobis(di-tert-butylphosphinito-kP)palladate). The reaction mixture was heated to reflux after 4 h. The solution was concentrated, diluted with ethyl acetate, filtered and washed with ethyl acetate. The filtrate was washed with brine, dried and evaporated. Column chromatography (silica gel, 20-60% E/H) gave methyl 4-(2-methylpyridin-4-yl)benzoate.

### Step 2

Methyl 4-(2-methylpyridin-4-yl)benzoate (8.3 g, 37 mmol) in 5 N HCl (150 mL) was heated at 100° C. for 11 h. The reaction mixture was evaporated and dried to give 4-(2-methylpyridin-4-yl)benzoic acid hydrochloride (8.8 g) as a white solid.

### Step 3

A mixture of 4-(2-methylpyridin-4-yl)benzoic acid hydrochloride (1 g, 4 mmol) in dry THF (15 mL) was cooled to 0° C. with stirring. TEA (1 mL, 8 mmol) was added and the reaction mixture was stirred for 20 min. Ethyl chloroformate (0.4 mL, 4 mmol) was added and the solution was stirred for 30 min. A solution of sodium azide (0.3 g, 4 mmol) in H₂O (1 mL) was added and the reaction mixture was allowed to warm to room temperature. After 2 h, 3 mL of H₂O was added and the mixture was extracted with EtOAc, dried (MgSO₄) and concentrated in vacuo to give the title compound as a light-yellow solid.

Following the procedure described above and using appropriate starting materials azido(3-fluoro-4-(2-methylpyridin-4-yl)phenyl)methanone, azido(3-methyl-4-(2-methylpyridin-4-yl)phenyl)methanone, azido(3-methoxy-4-(2-methylpyridin-4-yl)phenyl)methanone, azido(1-phenylcyclohexyl)methanone, azido(4-(2-fluorophenyl)-tetrahydro-2H-pyran-4-yl)methanone, and tert-butyl 4-(azidocarbonyl)-4-(2-fluorophenyl)piperidine-1-carboxylate, azido(1-phenylcyclopentyl)methanone, azido(1-phenylcyclopropyl)methanone were synthesized.

### Reference 5

### Synthesis of 6-(2-methylpyridin-4-yl)pyridin-3-amine

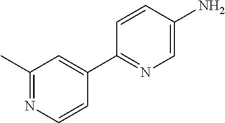

A mixture of potassium phosphate hydrate (4.5 g, 20 mmol), 2-methylpyridin-4-ylboronic acid (1.8 g, 13 mmol), 6-bromopyridin-3-amine (1.50 g, 8.7 mmol) in dioxane (50 mL) and water (7 mL) was purged with nitrogen and then bis(di-t-butylphenylphosphine)-dichloropalladium catalyst (0.27 g, 0.43 mmol) (see Organic letters, 8(9), 1787-1789 (2006) for the catalyst) was added. The reaction mixture was heated to 100° C. for 4 h. HPLC-MS showed the product (80%) and the bromide (20%). The reaction was stirred overnight. HPLC-MS showed no more bromide left. The reaction mixture was diluted with EtOAc (200 mL), washed with saturated Na₂CO₃, and brine. The organic phase was dried over Na₂SO₄, filtered and concentrated in vacuo. Purification by flash column chromatography (silica gel, 5-10% MeOH—CH₂Cl₂) afforded the title compound as orange oil, which turned to solid upon standing. It was triturated with EtOAc-Hexane to give an orange solid by filtration. MS: 186 (M+1).

### Example 1

### Synthesis of (R)-1-(4-(2-methylpyridin-4-yl)phenyl)-3-(1-(naphthalen-2-yl)ethyl)urea

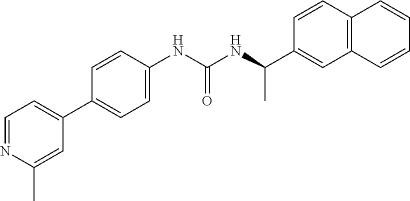

A 2 mL microwave synthesizer vessel containing a solution of azido(4-(2-methylpyridin-4-yl)phenyl)methanone (0.187 g, 0.78 mmol) in THF (1.2 mL) was subjected to microwave irradiation at 120° C. for 15 min. S(−)-1-(2-Naphthyl)ethylamine (0.13 g, 0.78 mmol) and N,N-diisopropylethylamine (0.14 mL, 0.78 mmol) were added and the reaction mixture was subjected to microwave irradiation at 120° C. for an additional 15 min. The reaction mixture was transferred to a scintillation vial, washed with methanol (5 mL). The solvent was removed in vacuo and the residue was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give (R)-1-(4-(2-methylpyridin-4-yl)phenyl)-3-(1-(naphthalen-2-yl)ethyl)urea as a light-yellow solid. MS (ESI pos. ion) m/z: 382 (M+1).

Proceeding as described in Example 1 above, but substituting S(−)-1-(2-naphthyl)-ethylamine with other commercially available amine, the following compounds were synthesized.

| | | | MS: ESI |
|---|---|---|---|
| | | | m/z |
| Ex | Structure | Name | (M + 1) |
| 2 | 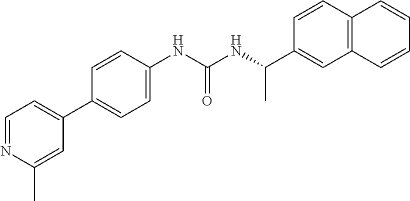 | 1-(4-(2-methyl-4-pyridinyl)phenyl)-3- ((1S)-1-(2-naphthalenyl)ethyl)urea | 382 |
| 3 | 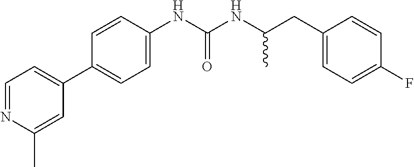 | 1-((1S)-2-(4-fluorophenyl)-1- methylethyl)-3-(4-(2-methyl-4- pyridinyl)phenyl)urea | 364 |
| 4 | 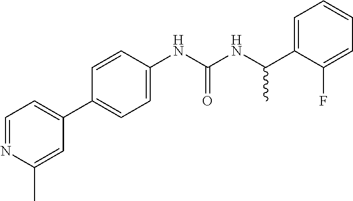 | 1-((1S)-1-(2-fluorophenyl)ethyl)-3- (4-(2-methyl-4-pyridinyl)phenyl)urea | 350 |
| 5 | 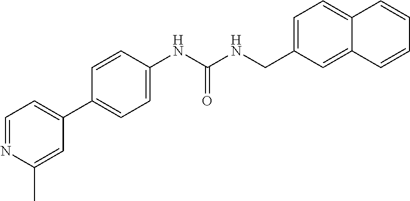 | 1-(4-(2-methyl-4-pyridinyl)phenyl)-3- (2-naphthalenylmethyl)urea | 368 |
| 6 | 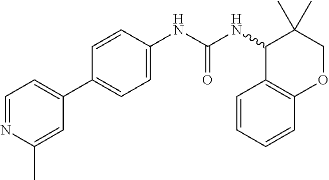 | 1-((4R/S)-3,3-dimethyl-3,4-dihydro- 2H-chromen-4-yl)-3-(4-(2-methyl-4- pyridinyl)phenyl)urea | 388 |

### Example 7

### Synthesis of 1-(1-(3-fluorophenyl)ethyl)-3-(4-(2-methylpyridin-4-yl)phenyl)urea

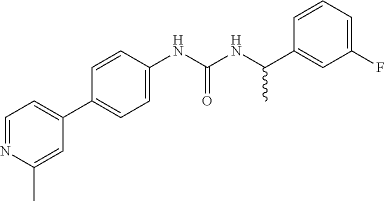

A 2 mL microwave synthesizer vessel containing a solution of azido(4-(2-methylpyridin-4-yl)phenyl)methanone (0.205 g, 0.860 mmol) in THF (2.0 mL) was subjected to a microwave irradiation at 120° C. for 15 min. 1-(3-fluorophenyl)ethanamine (0.270 g, 1.94 mmol) and N,N-diisopropylethylamine (0.300 mL, 1.72 mmol) were added and the reaction mixture was stirred at room temperature for 12 h. The reaction mixture was transferred to a scintillation vial, washed with methanol (5 mL). The solvent was removed in vacuo and the residue was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(1-(3-fluorophenyl)ethyl)-3-(4-(2-methylpyridin-4-yl)phenyl)urea as a colorless oil. MS (ESI pos. ion) m/z: 350 (M+1).

Proceeding as described in Example 7 above but using azido(4-(2-methylpyridin-4-yl)phenyl)methanone and the corresponding commercially available amine, the following compounds were prepared.

| | | | MS: ESI |
|---|---|---|---|
| | | | m/z |
| Ex. | Structure | Name | (M + 1) |
| 8 | 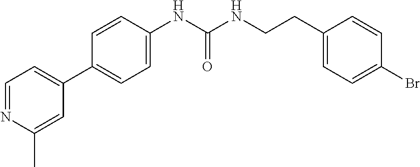 | 1-(2-(4-bromophenyl)ethyl)-3-(4- (2-methyl-4-pyridinyl)phenyl)urea | 410 |
| 9 | 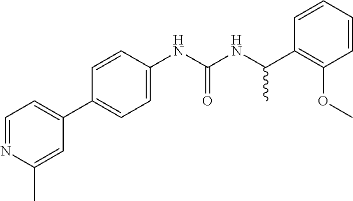 | 1-((1R)-1-(2- methoxyphenyl)ethyl)-3-(4-(2- methyl-4-pyridinyl)phenyl)urea | 362 |
| 10 | 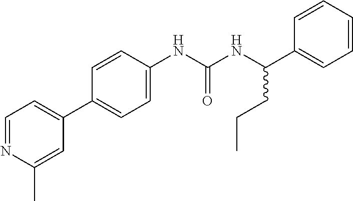 | 1-(4-(2-methyl-4-pyridinyl)phenyl)- 3-((1R)-1-phenylbutyl)urea | 360 |
| 11 | 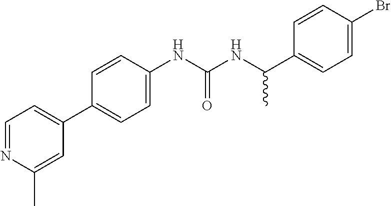 | 1-((1S)-1-(4-bromophenyl)ethyl)-3- (4-(2-methyl-4- pyridinyl)phenyl)urea | 410 |
| 12 | 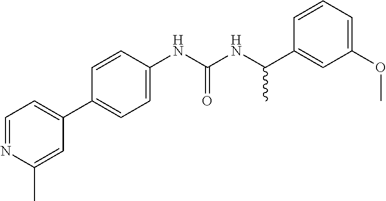 | 1-(1-(3-methoxyphenyl)ethyl)-3-(4- (2-methyl-4-pyridinyl)phenyl)urea | 362 |
| 13 | 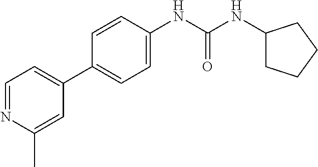 | 1-cyclopentyl-3-(4-(2-methyl-4- pyridinyl)phenyl)urea | 296 |
| 14 | 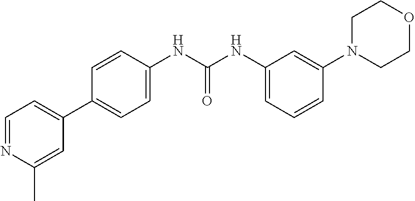 | 1-(4-(2-methylpyridin-4-yl)phenyl)- 3-(3-morpholinophenyl)urea | 389 |
| 15 | 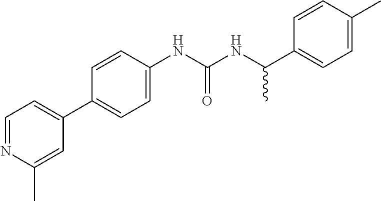 | 1-(4-(2-methylpyridin-4-yl)phenyl)- 3-(1-p-tolylethyl)urea | 346 |
| 16 | 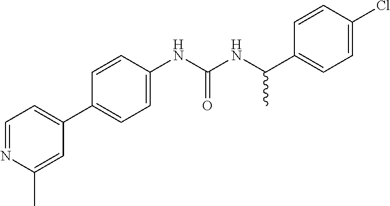 | 1-(1-(4-chlorophenyl)ethyl)-3-(4- (2-methylpyridin-4-yl)phenyl)urea | 366 |
| 17 | 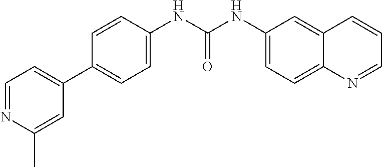 | 1-(4-(2-methylpyridin-4-yl)phenyl)- 3-(quinolin-6-yl)urea | 355 |
| 18 | 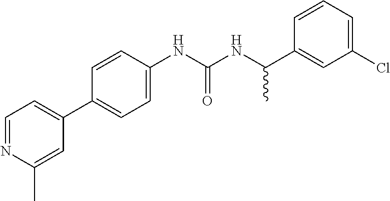 | 1-(1-(3-chlorophenyl)ethyl)-3-(4- (2-methylpyridin-4-yl)phenyl)urea | 366 |
| 19 | 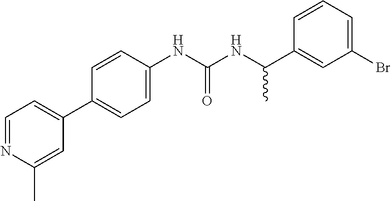 | 1-(1-(3-bromophenyl)ethyl)-3-(4- (2-methylpyridin-4-yl)phenyl)urea | 410 |
| 20 | 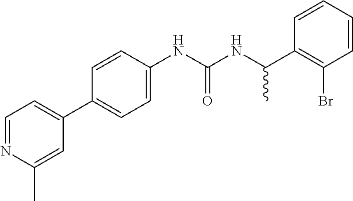 | 1-(1-(2-bromophenyl)ethyl)-3-(4- (2-methylpyridin-4-yl)phenyl)urea | 410 |
| 21 | 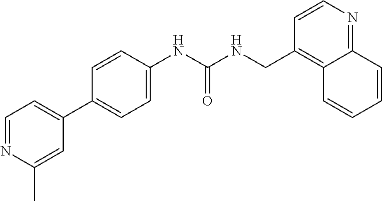 | 1-(4-(2-methylpyridin-4-yl)phenyl)- 3-(quinolin-4-ylmethyl)urea | 369 |
| 22 | 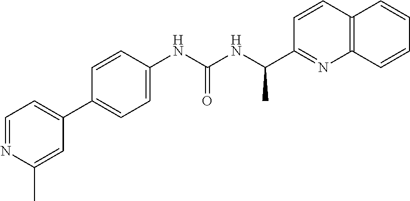 | (R)-1-(4-(2-methylpyridin-4- yl)phenyl)-3-(1-(quinolin-2- yl)ethyl)urea | 383 |
| 23 | 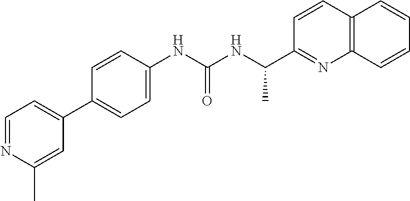 | (S)-1-(4-(2-methylpyridin-4- yl)phenyl)-3-(1-(quinolin-2- yl)ethyl)urea | 383 |
| 24 | 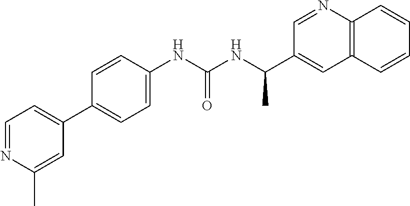 | (R)-1-(4-(2-methylpyridin-4- yl)phenyl)-3-(1-(quinolin-3- yl)ethyl)urea | 383 |
| 25 | 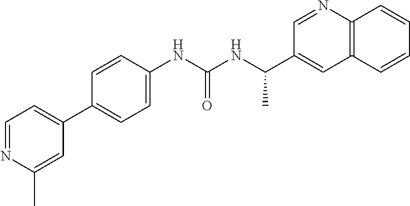 | (S)-1-(4-(2-methylpyridin-4- yl)phenyl)-3-(1-(quinolin-3- yl)ethyl)urea | 383 |
| 26 | 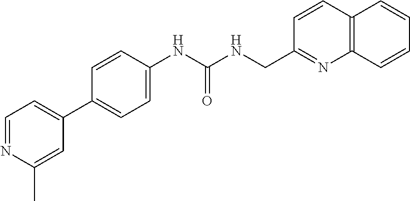 | 1-(4-(2-methylpyridin-4-yl)phenyl)- 3-(quinolin-2-ylmethyl)urea | 369 |
| 27 | 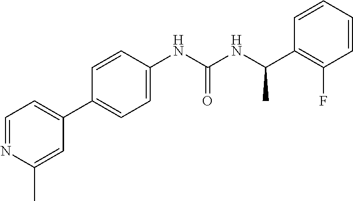 | (R)-1-(1-(2-fluorophenyl)ethyl)-3- (4-(2-methylpyridin-4- yl)phenyl)urea | 350 |
| 28 | 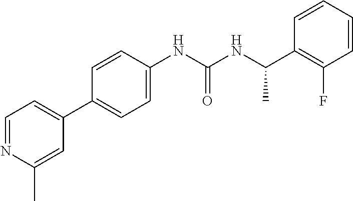 | (S)-1-(1-(2-fluorophenyl)ethyl)-3- (4-(2-methyl pyridin-4- yl)phenyl)urea | 350 |
| 29 | 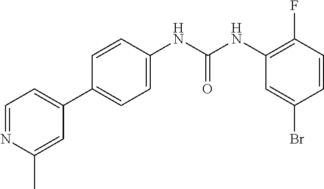 | 1-(5-bromo-2-fluorophenyl)-3-(4- (2-methylpyridin-4-yl)phenyl)urea | 400 |
| 30 | 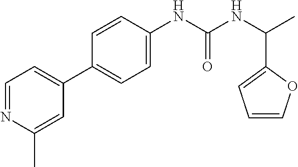 | 1-(1-(furan-2-yl)ethyl)-3-(4-(2- methylpyridin-4-yl)phenyl)urea | 322 |
| 31 | 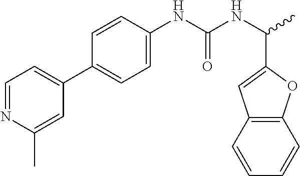 | 1-(1-(benzofuran-2-yl)ethyl)-3-(4- (2-methylpyridin-4-yl)phenyl)urea | 372 |
| 32 | 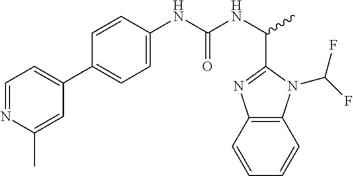 | 1-(1-(1-(difluoromethyl)-1H- benzo[d]imidazol-2-yl)ethyl)-3-(4- (2-methylpyridin-4-yl)phenyl)urea | 422 |
| 33 | 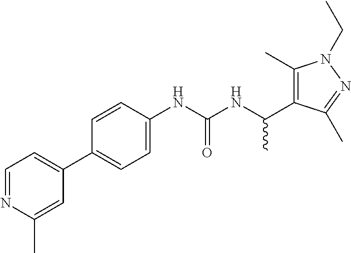 | 1-(1-(1-ethyl-3,5-dimethyl-1H- pyrazol-4-yl)ethyl)-3-(4-(2- methylpyridin-4-yl)phenyl)urea | 378 |
| 34 | 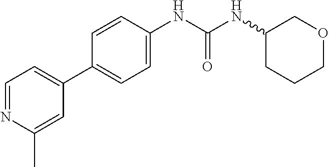 | 1-(4-(2-methylpyridin-4-yl)phenyl)- 3-(tetrahydro-2H-pyran-3-yl)urea | 312 |
| 35 | 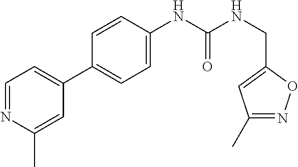 | 1-((3-methylisoxazol-5-yl)methyl)- 3-(4-(2-methylpyridin-4- yl)phenyl)urea | 323 |
| 36 | 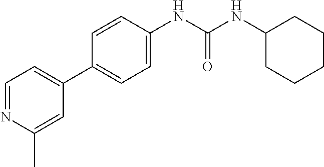 | 1-cyclohexyl-3-(4-(2-methyl-4- pyridinyl)phenyl)urea | 310 |
| 37 | 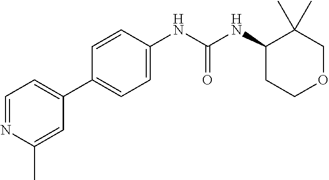 | (R)-1-(3,3-dimethyltetrahydro-2H- pyran-4-yl)-3-(4-(2-methylpyridin- 4-yl)phenyl)urea | 340 |
| 38 | 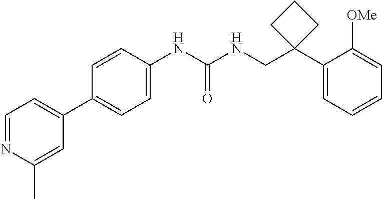 | 1-((1-(2- methoxyphenyl)cyclobutyl)methyl)- 3-(4-(2-methylpyridin-4- yl)phenyl)urea | 402 |
| 39 | 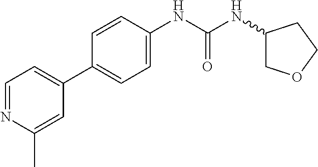 | 1-(4-(2-methylpyridin-4-yl)phenyl)- 3-(tetrahydrofuran-3-yl)urea | 298 |
| 40 | 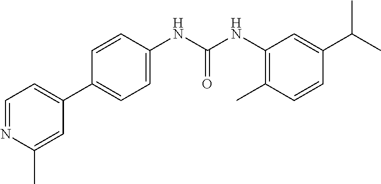 | 1-(2-methyl-5-(1- methylethyl)phenyl)-3-(4-(2- methyl-4-pyridinyl)phenyl)urea | 360 |
| 41 | 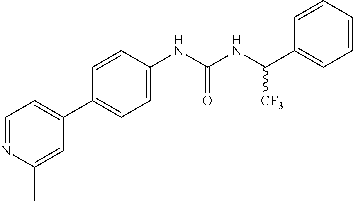 | 1-(4-(2-methylpyridin-4-yl)phenyl)- 3-(2,2,2-trifluoro-1- phenylethyl)urea | 386 |
| 42 | 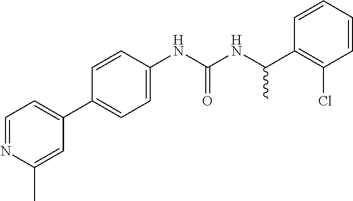 | 1-(1-(2-chlorophenyl)ethyl)-3-(4- (2-methylpyridin-4-yl)phenyl)urea | 366 |
| 43 | 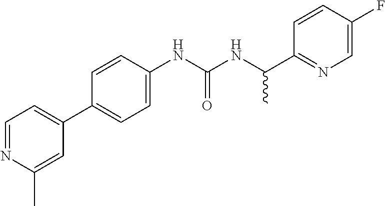 | 1-(4-(2-methylpyridin-4-yl)phenyl)- 3-(1-o-tolylethyl)urea | 346 |
| 44 | 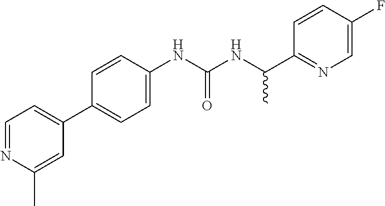 | 1-(1-(5-fluoropyridin-2-yl)ethyl)-3- (4-(2-methylpyridin-4- yl)phenyl)urea | 351 |
| 45 | 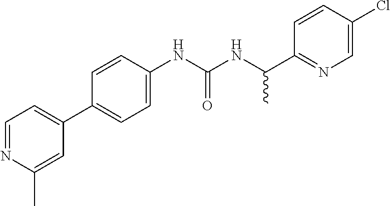 | 1-(1-(5-chloropyridin-2-yl)ethyl)-3- (4-(2-methylpyridin-4- yl)phenyl)urea | 367 |
| 46 | 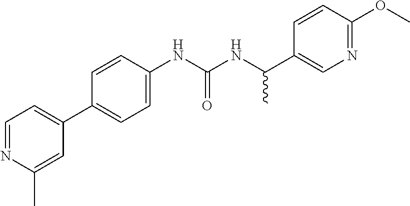 | 1-(1-(6-methoxypyridin-3-yl)ethyl)- 3-(4-(2-methylpyridin-4- yl)phenyl)urea | 363 |
| 47 | 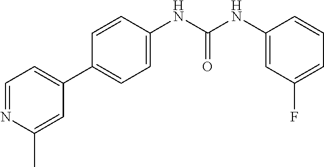 | 1-(3-fluorophenyl)-3-(4-(2-methyl- 4-pyridinyl)phenyl)urea | 322 |
| 48 | 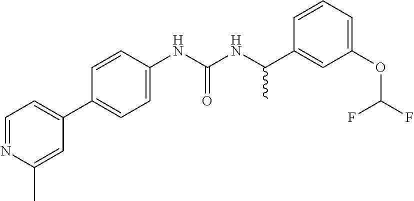 | 1-(1-(3- (difluoromethoxy)phenyl)ethyl)-3- (4-(2-methylpyridin-4- yl)phenyl)urea | 398 |
| 49 | 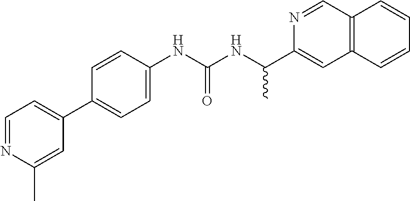 | 1-(1-(isoquinolin-3-yl)ethyl)-3-(4- (2-methylpyridin-4-yl)phenyl)urea | 383 |
| 50 | 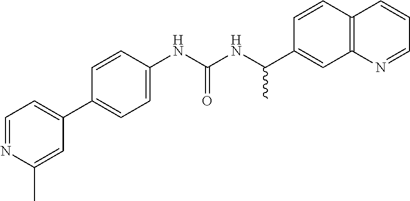 | 1-(4-(2-methylpyridin-4-yl)phenyl)- 3-(1-(quinolin-7-yl)ethyl)urea | 383 |
| 51 | 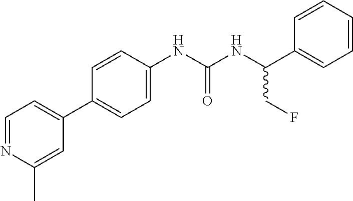 | 1-(2-fluoro-1-phenylethyl)-3-(4-(2- methylpyridin-4-yl)phenyl)urea | 350 |
| 52 | 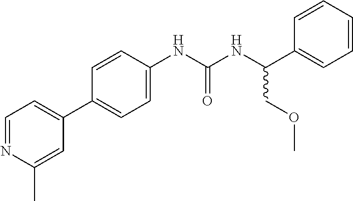 | 1-(2-methoxy-1-phenylethyl)-3-(4- (2-methylpyridin-4-yl)phenyl)urea | 362 |
| 53 | 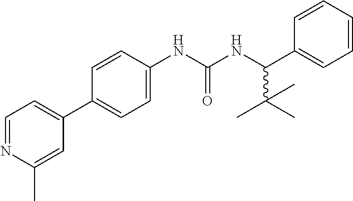 | 1-(2-fluoro-2-methyl-1- phenylpropyl)-3-(4-(2- methylpyridin-4-yl)phenyl)urea | 378 |
| 54 | 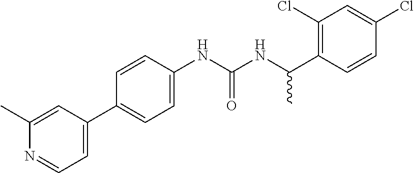 | 1-(1-(2,4-dichlorophenyl)ethyl)-3- (4-(2-methylpyridin-4- yl)phenyl)urea | 400 |
| 55 | 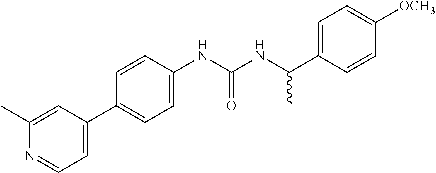 | 1-(1-(4-methoxyphenyl)ethyl)-3-(4- (2-methylpyridin-4-yl)phenyl)urea | 362 |
| 56 | 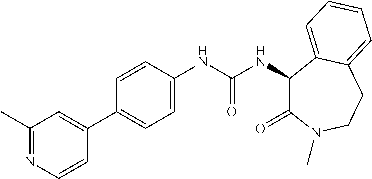 | (S)-1-(3-methyl-2-oxo-2,3,4,5- tetrahydro-1H-benzo[d]azepin-1- yl)-3-(4-(2-methylpyridin-4- yl)phenyl)urea | 401 |
| 57 | 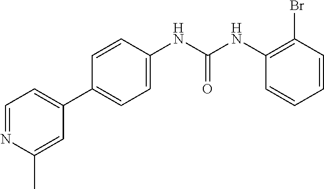 | 1-(2-bromophenyl)-3-(4-(2-methyl- 4-pyridinyl)phenyl)urea | 382 |
| 58 | 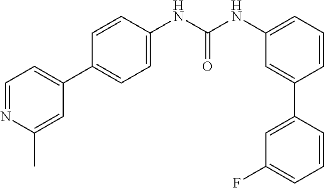 | 1-(3′-fluoro-3-biphenylyl)-3-(4-(2- methyl-4-pyridinyl)phenyl)urea | 398 |
| 59 | 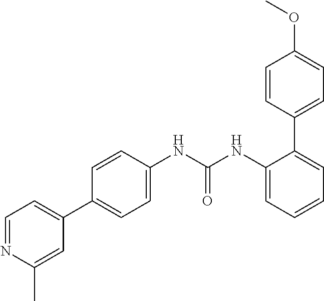 | 1-(4′-methoxy-2-biphenylyl)-3-(4- (2-methyl-4-pyridinyl)phenyl)urea | 410 |
| 60 | 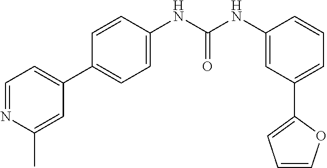 | 1-(3-(2-furanyl)phenyl)-3-(4-(2- methyl-4-pyridinyl)phenyl)urea | 370 |
| 61 | 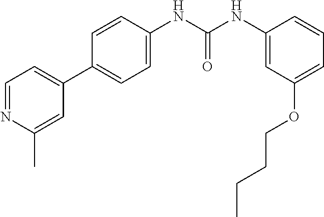 | 1-(3-butoxyphenyl)-3-(4-(2-methyl- 4-pyridinyl)phenyl)urea | 376 |
| 62 | 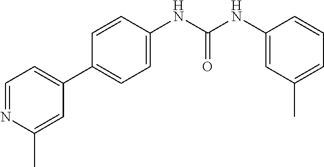 | 1-(3-methylphenyl)-3-(4-(2-methyl- 4-pyridinyl)phenyl)urea | 318 |
| 63 | 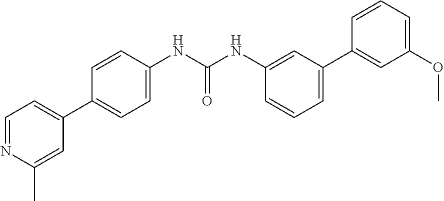 | 1-(3′-methoxy-3-biphenylyl)-3-(4- (2-methyl-4-pyridinyl)phenyl)urea | 410 |
| 64 | 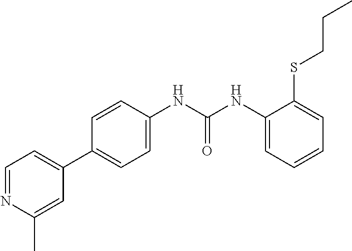 | 1-(4-(2-methyl-4-pyridinyl)phenyl)- 3-(2-(propylsulfanyl)phenyl)urea | 378 |
| 65 | 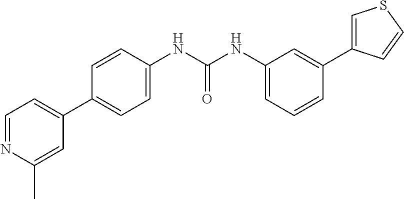 | 1-(4-(2-methyl-4-pyridinyl)phenyl)- 3-(3-(3-thiophenyl)phenyl)urea | 386 |
| 66 | 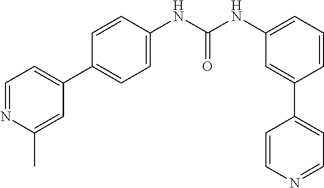 | 1-(4-(2-methyl-4-pyridinyl)phenyl)- 3-(3-(4-pyridinyl)phenyl)urea | 381 |
| 67 | 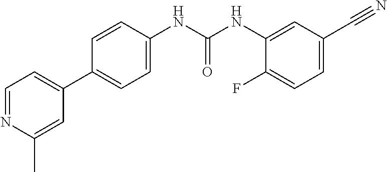 | 1-(5-cyano-2-fluorophenyl)-3-(4- (2-methyl-4-pyridinyl)phenyl)urea | 347 |
| 68 | 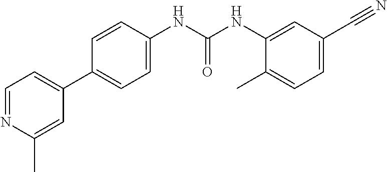 | 1-(5-cyano-2-methylphenyl)-3-(4- (2-methyl-4-pyridinyl)phenyl)urea | 343 |
| 69 | 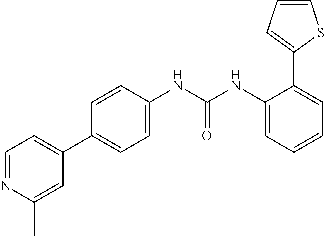 | 1-(4-(2-methyl-4-pyridinyl)phenyl)- 3-(2-(2-thiophenyl)phenyl)urea | 386 |
| 70 | 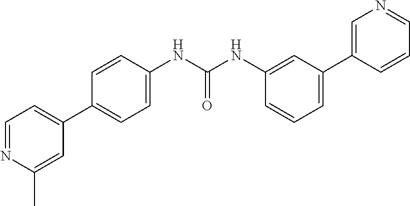 | 1-(4-(2-methyl-4-pyridinyl)phenyl)- 3-(3-(3-pyridinyl)phenyl)urea | 381 |
| 71 | 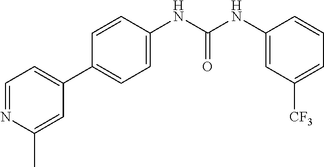 | 1-(4-(2-methyl-4-pyridinyl)phenyl)- 3-(3-(trifluoromethyl)phenyl)urea | 372 |
| 72 | 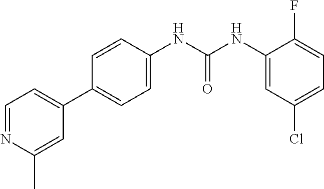 | 1-(5-chloro-2-fluorophenyl)-3-(4- (2-methyl-4-pyridinyl)phenyl)urea | 356 |
| 73 | 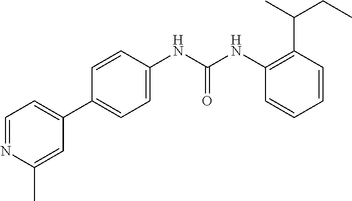 | 1-(2-(1-methylpropyl)phenyl)-3-(4- (2-methyl-4-pyridinyl)phenyl)urea | 360 |
| 74 | 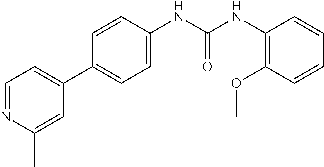 | 1-(2-methoxyphenyl)-3-(4-(2- methyl-4-pyridinyl)phenyl)urea | 334 |
| 75 | 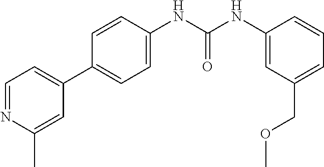 | 1-(3-(methoxymethyl)phenyl)-3-(4- (2-methyl-4-pyridinyl)phenyl)urea | 348 |
| 76 | 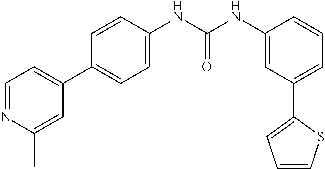 | 1-(4-(2-methyl-4-pyridinyl)phenyl)- 3-(3-(2-thiophenyl)phenyl)urea | 386 |
| 77 | 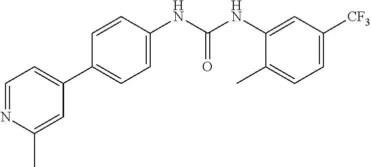 | 1-(4-(2-methyl-4-pyridinyl)phenyl)- 3-(2-methyl-5- (trifluoromethyl)phenyl)urea | 386 |
| 78 | 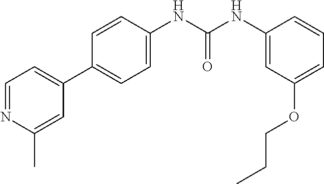 | 1-(4-(2-methyl-4-pyridinyl)phenyl)- 3-(3-propoxyphenyl)urea | 362 |
| 79 | 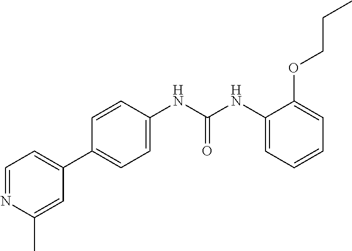 | 1-(4-(2-methyl-4-pyridinyl)phenyl)- 3-(2-propoxyphenyl)urea | 362 |
| 80 | 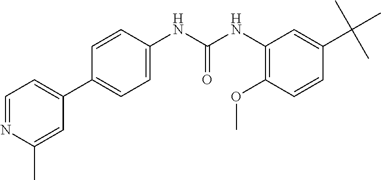 | 1-(5-tert-butyl-2-methoxyphenyl)- 3-(4-(2-methyl-4- pyridinyl)phenyl)urea | 390 |
| 81 | 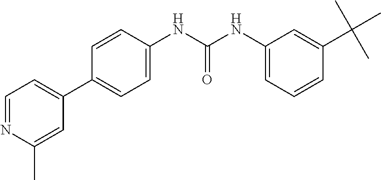 | 1-(3-tert-butylphenyl)-3-(4-(2- methyl-4-pyridinyl)phenyl)urea | 360 |
| 83 | 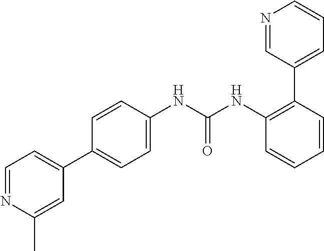 | 1-(4-(2-methyl-4-pyridinyl)phenyl)- 3-(2-(3-pyridinyl)phenyl)urea | 381 |
| 84 | 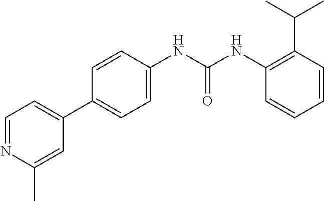 | 1-(2-(1-methylethyl)phenyl)-3-(4- (2-methyl-4-pyridinyl)phenyl)urea | 346 |
| 85 | 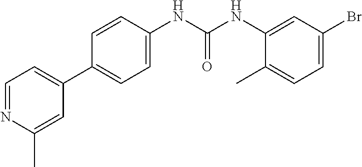 | 1-(5-bromo-2-methylphenyl)-3-(4- (2-methyl-4-pyridinyl)phenyl)urea | 396 |
| 86 | 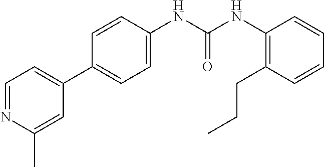 | 1-(4-(2-methyl-4-pyridinyl)phenyl)- 3-(2-propylphenyl)urea | 346 |
| 87 | 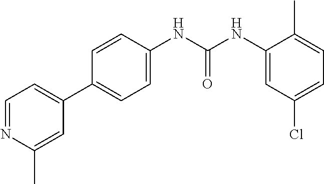 | 1-(5-chloro-2-methylphenyl)-3-(4- (2-methyl-4-pyridinyl)phenyl)urea | 352 |
| 88 | 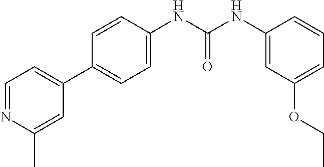 | 1-(3-ethoxyphenyl)-3-(4-(2-methyl- 4-pyridinyl)phenyl)urea | 348 |
| 89 | 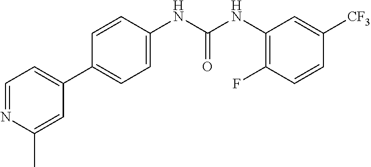 | 1-(2-fluoro-5- (trifluoromethyl)phenyl)-3-(4-(2- methyl-4-pyridinyl)phenyl)urea | 390 |
| 90 | 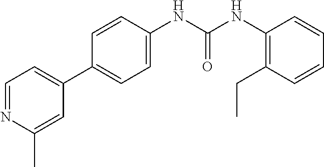 | 1-(2-ethylphenyl)-3-(4-(2-methyl-4- pyridinyl)phenyl)urea | 332 |
| 91 | 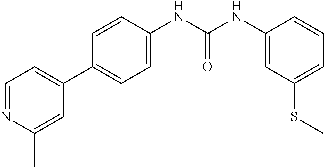 | 1-(4-(2-methyl-4-pyridinyl)phenyl)- 3-(3-(methylsulfanyl)phenyl)urea | 350 |
| 92 | 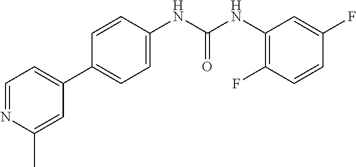 | 1-(2,5-difluorophenyl)-3-(4-(2- methyl-4-pyridinyl)phenyl)urea | 340 |
| 93 | 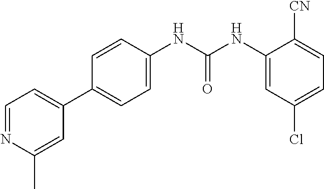 | 1-(5-chloro-2-cyanophenyl)-3-(4- (2-methyl-4-pyridinyl)phenyl)urea | 363 |
| 94 | 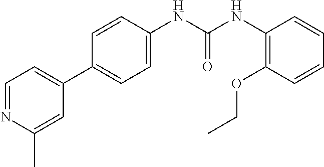 | 1-(2-ethoxyphenyl)-3-(4-(2-methyl- 4-pyridinyl)phenyl)urea | 348 |
| 95 | 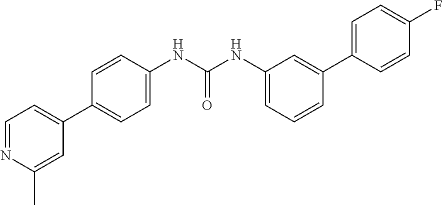 | 1-(4′-fluoro-3-biphenylyl)-3-(4-(2- methyl-4-pyridinyl)phenyl)urea | 398 |
| 96 | 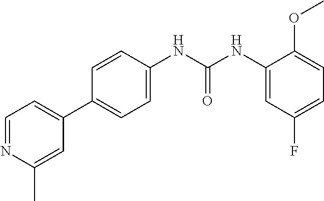 | 1-(5-fluoro-2-methoxyphenyl)-3-(4- (2-methyl-4-pyridinyl)phenyl)urea | 352 |
| 97 | 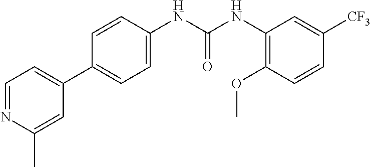 | 1-(2-methoxy-5- (trifluoromethyl)phenyl)-3-(4-(2- methyl-4-pyridinyl)phenyl)urea | 402 |
| 98 | 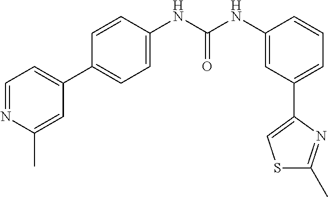 | 1-(4-(2-methyl-4-pyridinyl)phenyl)- 3-(3-(2-methyl-1,3-thiazol-4- yl)phenyl)urea | 401 |
| 99 | 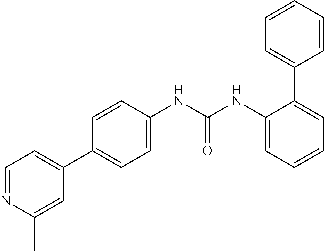 | 1-(2-biphenylyl)-3-(4-(2-methyl-4- pyridinyl)phenyl)urea | 380 |
| 100 | 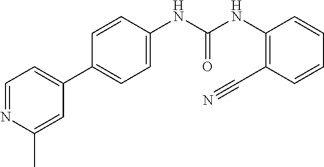 | 1-(2-cyanophenyl)-3-(4-(2-methyl- 4-pyridinyl)phenyl)urea | 329 |
| 101 | 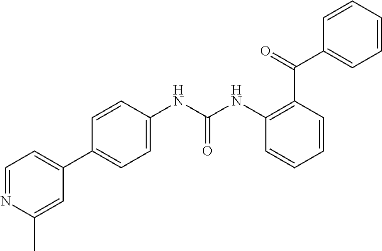 | 1-(4-(2-methyl-4-pyridinyl)phenyl)- 3-(2-(phenylcarbonyl)phenyl)urea | 408 |
| 102 | 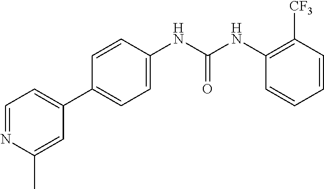 | 1-(4-(2-methyl-4-pyridinyl)phenyl)- 3-(2-(trifluoromethyl)phenyl)urea | 372 |
| 103 | 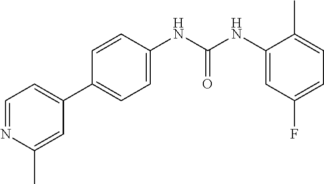 | 1-(5-fluoro-2-methylphenyl)-3-(4- (2-methyl-4-pyridinyl)phenyl)urea | 336 |
| 104 | 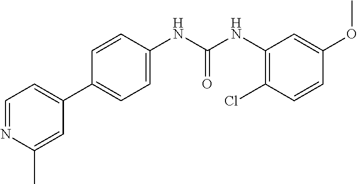 | 1-(2-chloro-5-methoxyphenyl)-3- (4-(2-methyl-4- pyridinyl)phenyl)urea | 368 |
| 105 | 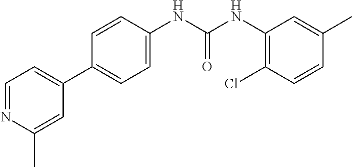 | 1-(2-chloro-5-methylphenyl)-3-(4- (2-methyl-4-pyridinyl)phenyl)urea | 352 |
| 106 | 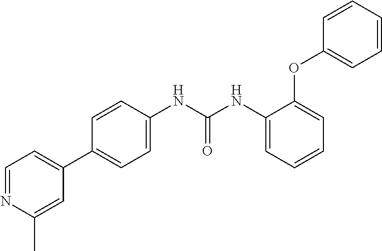 | 1-(4-(2-methyl-4-pyridinyl)phenyl)- 3-(2-phenoxyphenyl)urea | 396 |
| 107 | 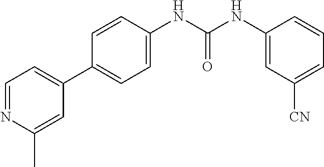 | 1-(3-cyanophenyl)-3-(4-(2-methyl- 4-pyridinyl)phenyl)urea | 329 |
| 108 | 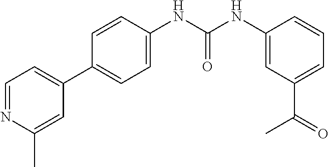 | 1-(3-acetylphenyl)-3-(4-(2-methyl- 4-pyridinyl)phenyl)urea | 346 |
| 109 | 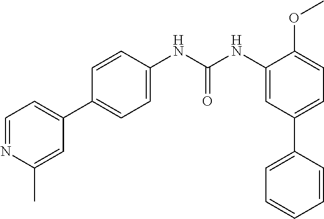 | 1-1-(4-methoxybiphenyl-3-yl)-3-(4- (2-methylpyridin-4-yl)phenyl)urea | 410 |
| 110 | 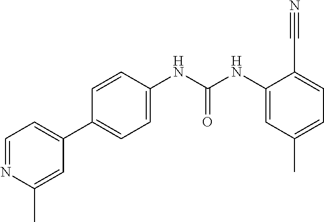 | 1-(2-cyano-5-methylphenyl)-3-(4- (2-methyl-4-pyridinyl)phenyl)urea | 343 |
| 111 | 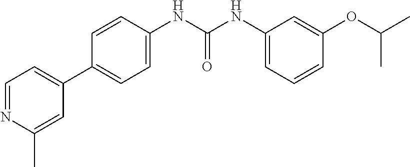 | 1-(3-(1-methylethoxy)phenyl)-3-(4- (2-methyl-4-pyridinyl)phenyl)urea | 362 |
| 112 | 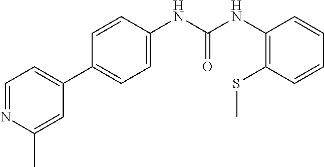 | 1-(4-(2-methyl-4-pyridinyl)phenyl)- 3-(2-(methylsulfanyl)phenyl)urea | 350 |
| 113 | 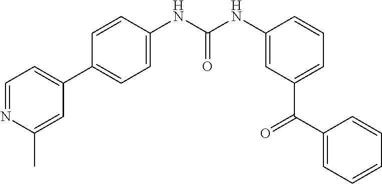 | 1-(4-(2-methyl-4-pyridinyl)phenyl)- 3-(3-(phenylcarbonyl)phenyl)urea | 408 |
| 114 | 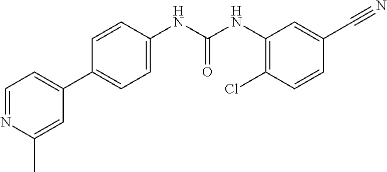 | 1-(2-chloro-5-cyanophenyl)-3-(4- (2-methyl-4-pyridinyl)phenyl)urea | 363 |
| 115 | 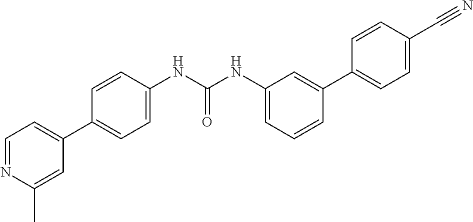 | 1-(4′-cyano-3-biphenylyl)-3-(4-(2- methyl-4-pyridinyl)phenyl)urea | 405 |
| 116 | 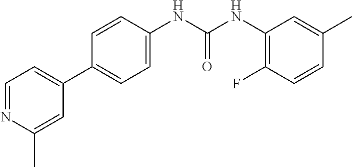 | 1-(2-fluoro-5-methylphenyl)-3-(4- (2-methyl-4-pyridinyl)phenyl)urea | 336 |
| 117 | 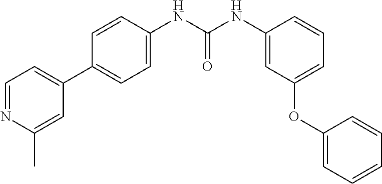 | 1-(4-(2-methyl-4-pyridinyl)phenyl)- 3-(3-phenoxyphenyl)urea | 396 |
| 118 | 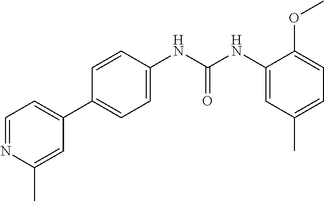 | 1-(2-methoxy-5-methylphenyl)-3- (4-(2-methyl-4- pyridinyl)phenyl)urea | 348 |
| 119 | 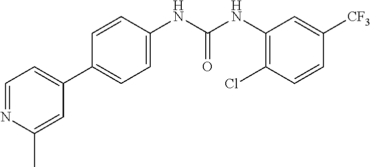 | 1-(2-chloro-5- (trifluoromethyl)phenyl)-3-(4-(2- methyl-4-pyridinyl)phenyl)urea | 406 |
| 120 | 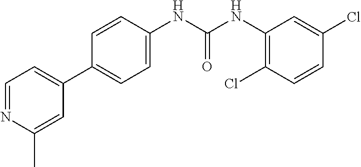 | 1-(2,5-dichlorophenyl)-3-(4-(2- methyl-4-pyridinyl)phenyl)urea | 372 |
| 121 | 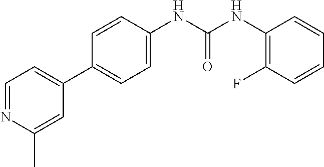 | 1-(2-fluorophenyl)-3-(4-(2-methyl- 4-pyridinyl)phenyl)urea | 322 |
| 122 | 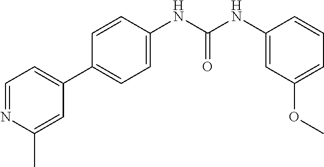 | 1-(3-methoxyphenyl)-3-(4-(2- methyl-4-pyridinyl)phenyl)urea | 334 |
| 123 | 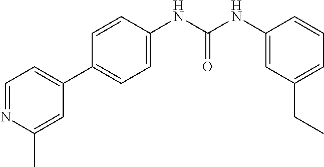 | 1-(3-ethylphenyl)-3-(4-(2-methyl-4- pyridinyl)phenyl)urea | 332 |
| 124 | 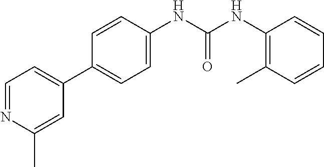 | 1-(2-methyl phenyl)-3-(4-(2-methyl- 4-pyridinyl)phenyl)urea | 318 |

### Example 125

### Synthesis of 1-(4-(2-methylpyridin-4-yl)phenyl)-3-((1R,2S)-2-phenylcyclopropyl)urea

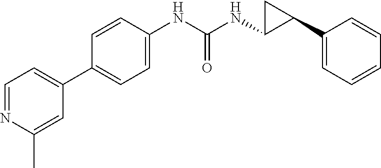

A 2 mL microwave synthesizer vessel containing a suspension of 4-(2-methylpyridin-4-yl)benzenamine (0.207 g, 1.12 mmol) in 1,2-dichloroethane (2.5 mL) was treated with N,N-diisopropylethylamine (0.260 mL, 1.49 mmol) followed by trans-2-phenylcyclopropyl isocyanate (0.170 mL, 1.15 mmol). The resulting suspension was stirred at room temperature for 16 h. The reaction mixture was transferred to a scintillation vial, washed with methanol (5 mL). The solvent was removed in vacuo and the residue was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(4-(2-methylpyridin-4-yl)phenyl)-3-((1R,2S)-2-phenylcyclopropyl)urea as an amorphous off-white solid. MS (ESI pos. ion) m/z: 344 (M+1).

Proceeding as described in Example 125 above, but using 4-(2-methylpyridin-4-yl)benzenamine and the corresponding commercially available isocyanate, the following compounds were synthesized.

| | | | MS: ESI |
|---|---|---|---|
| | | | m/z |
| Examples | Structure | Name | (M + 1) |
| 126 | 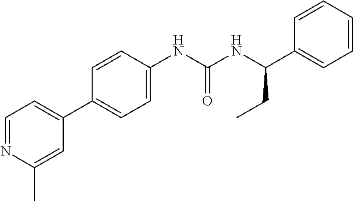 | 1-(4-(2-methyl-4- pyridinyl)phenyl)-3-((1R)-1- phenylpropyl)urea | 346 |
| 127 | 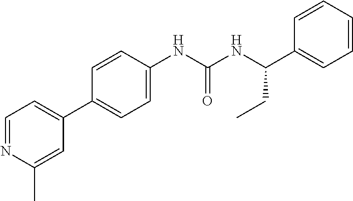 | 1-(4-(2-methyl-4- pyridinyl)phenyl)-3-((1S)-1- phenylpropyl)urea | 346 |
| 128 | 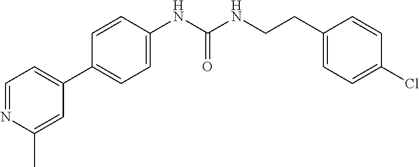 | 1-(2-(4-chlorophenyl)ethyl)-3- (4-(2-methyl-4- pyridinyl)phenyl)urea | 366 |
| 129 | 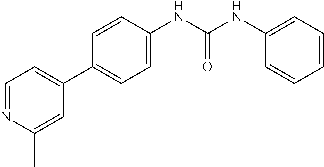 | 1-(4-(2-methyl pyridin-4- yl)phenyl)-3-phenylurea | 304 |
| 130 | 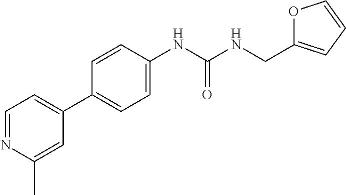 | 1-(furan-2-yl methyl)-3-(4-(2- methylpyridin-4-yl)phenyl)urea | 308 |
| 131 | 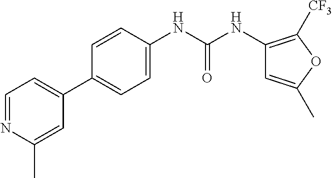 | 1-(5-methyl-2- (trifluoromethyl)furan-3-yl)-3-(4- (2-methylpyridin-4- yl)phenyl)urea | 376 |
| 132 | 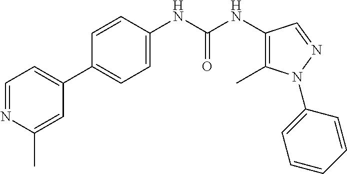 | 1-(5-methyl-1-phenyl-1H- pyrazol-4-yl)-3-(4-(2- methylpyridin-4-yl)phenyl)urea | 384 |
| 133 | 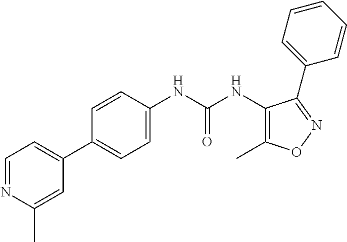 | 1-(5-methyl-3-phenylisoxazol- 4-yl)-3-(4-(2-methylpyridin-4- yl)phenyl)urea | 385 |
| 134 | 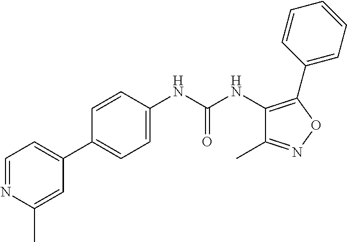 | 1-(3-methyl-5-phenylisoxazol- 4-yl)-3-(4-(2-methylpyridin-4- yl)phenyl)urea | 385 |
| 135 | 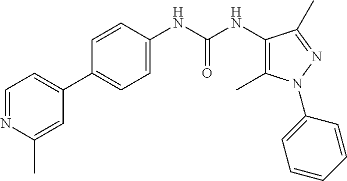 | 1-(3,5-dimethyl-1-phenyl-1H- pyrazol-4-yl)-3-(4-(2- methylpyridin-4-yl)phenyl)urea | 398 |
| 136 | 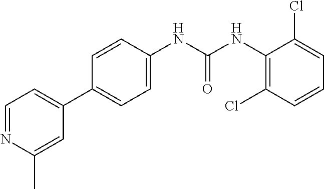 | 1-(2,6-dichlorophenyI)-3-(4-(2- methylpyridin-4-yl)phenyl)urea | 372 |
| 137 | 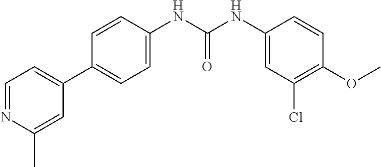 | 1-(3-chloro-4-methoxyphenyl)- 3-(4-(2-methyl-4- pyridinyl)phenyl)urea | 368 |
| 138 | 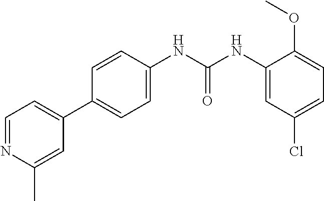 | 1-(5-chloro-2-methoxyphenyl)- 3-(4-(2-methyl-4- pyridinyl)phenyl)urea | 368 |
| 139 | 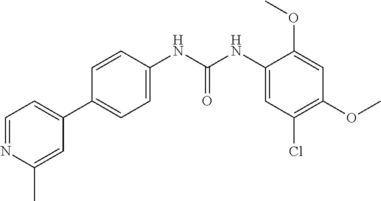 | 1-(5-chloro-2,4- dimethoxyphenyl)-3-(4-(2- methyl-4-pyridinyl)phenyl)urea | 398 |
| 140 | 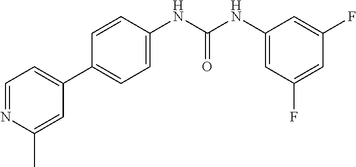 | 1-(3,5-difluorophenyl)-3-(4-(2- methyl-4-pyridinyl)phenyl)urea | 340 |

### Example 141

### Synthesis of 1-(4-cyanophenethyl)-3-(4-(2-methylpyridin-4-yl)phenyl)urea

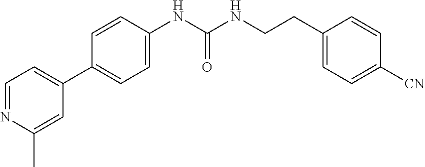

A 2 mL microwave synthesizer vessel containing a suspension of 1-(4-bromo-phenethyl)-3-(4-(2-methylpyridin-4-yl)phenyl)urea (0.356 g, 0.87 mmol), zinc cyanide (0.266 g, 2.3 mmol), and tetrakis(triphenylphosphine)palladium (0.064 g, 0.055 mmol) in N,N-dimethylformamide (2.3 mL) was heated to 110° C. for 15 min. The reaction mixture was filtered to remove the insoluble solids, washed with methanol (5 mL). The combined filtrates were transferred to a scintillation vial and the solvent was removed in vacuo. The resulting residue was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(4-cyanophenethyl)-3-(4-(2-methylpyridin-4-yl)phenyl)urea as an amorphous yellow solid. MS (ESI pos. ion) m/z: 357 (M+1).

### Example 142

### Synthesis of 1-(2-(4-bromothiophen-2-yl)propan-2-yl)-3-(4-(2-methylpyridin-4-yl)phenyl)urea

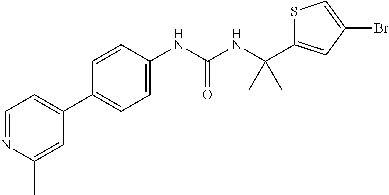

### Step 1

To a solution of 2-(4-bromothiophen-2-yl)acetic acid (553 mg, 2.501 mmol) in 5 mL of dry THF at 0° C. was added lithium bis(trimethylsilyl)amide (1.0M solution in tetrahydrofuran, 5.5 mL, 5.503 mmol). After stirring at 0° C. for 15 minutes, iodomethane (0.155 ml, 2.501 mmol) was added and the cold bath was removed. The reaction was stirred at RT for 16 h. Added more MeI (0.078 mL) and stirred at room temperature for another 6 h. It was quenched with 2 N HCl to pH ˜3.0, extracted with EtOAc, dried over Na₂SO₄, filtered and evaporated. The crude product was chromatographed through a Redi-Sep® pre-packed silica gel column (40 g), eluting with a gradient of 0% to 25% EtOAc in hexane, to provide 2-(4-bromothiophen-2-yl)propanoic acid as brown oil and was used without purification.

### Step 2

A 50 mL, round-bottomed flask containing a solution of 2-(4-bromothiophen-2-yl)-2-methylpropanoic acid (92 mg, 0.369 mmol) in THF (2.5 mL) at 0° C. was treated with triethylamine (0.154 ml, 1.108 mmol). The resulting mixture was stirred at 0° C. for 10 minutes. Then, ethyl chloroformate (0.071 mL, 0.739 mmol) was added and the mixture was stirred at 0° C. for an additional 60 minutes. Then, a solution of sodium azide (72 mg, 1.108 mmol) in water (0.3 mL) was added and the mixture was stirred at room temperature for 16 hours. Water (2 mL) was added and the mixture was extracted with EtOAc (2×30 mL). The combined organic extracts were dried over Na₂SO₄, concentrated, and dried in vacuum to give 2-(4-bromothiophen-2-yl)-2-methylpropanoyl azide as red oil and was used without purification.

### Step 3

A solution of 2-(4-bromothiophen-2-yl)-2-methylpropanoyl azide (0.10 g, 0.36 mmol) in THF (1 mL) was subjected to a microwave irradiation at 120° C. for 15 minutes. Then, 4-(2-methylpyridin-4-yl)benzenamine (0.074 g, 0.40 mmol) and n,n-diisopropylethylamine (0.15 mL, 0.84 mmol) were added and the mixture was subjected to a microwave irradiation at 120° C. for an additional 15 minutes. The crude mixture was concentrated to dryness. DCM (3 mL) was added and the resulting precipitate was collected by vacuum filtration, washed with DCM, dried to give 1-(2-(4-bromothiophen-2-yl)propan-2-yl)-3-(4-(2-methylpyridin-4-yl)phenyl)urea as off-white solid. MS (ESI pos. ion) m/z: 430 (M+1).

### Example 143

### Synthesis of 1-(1-(2-bromophenyl)-3-methylbutyl)-3-(4-(2-methylpyridin-4-yl)phenyl)urea

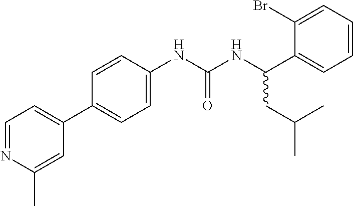

### Step 1

To a solution of 2-bromophenylacetic acid (2.15 g, 10 mmol) (azeotroped with toluene) in dry toluene (2 mL) of dry toluene was added sodium bis(trimethylsilyl)amide, (23 mL, 1.0 m solution in tetrahydrofuran). After stirring at room temperature for 20 minutes, 1-iodo-2-methylpropane (1 mL, 11 mmol) was added dropwise. After 10 minutes, the reaction was quenched with 2 N HCl to pH ˜2.0, extracted with ethyl acetate, dried over sodium sulfate, filtered and evaporated to dryness. The crude product was purified by silica gel flash column chromatography (eluted using 10%-100% EtOAc/dichloromethane gradient) to give the 2-(2-bromophenyl)-4-methylpentanoic acid as colorless oil. MS (ESI, positive ion) m/z: 271 (M+1).

### Step 2

A 100 mL, round-bottomed flask containing a suspension of 2-(2-bromophenyl)-4-methylpentanoic acid (0.315 g, 1.16 mmol) in THF (8 mL) at 0° C. was treated with triethylamine (0.405 mL, 2.91 mmol). The resulting mixture was stirred at 0° C. for 10 minutes. Then, ethyl chloroformate (0.150 mL, 1.57 mmol) was added and the mixture was stirred at 0° C. for an additional 60 minutes. Then, a solution of sodium azide (0.250 g, 3.85 mmol) in water (0.8 mL) was added and the mixture was stirred at room temperature for 16 hours. Then, H₂O (25 mL) was added and the mixture was extracted with EtOAc (2×50 mL). The combined organic extracts were dried over Na₂SO₄, concentrated, and dried in vacuum to give 2-(3-bromophenyl)propanoyl azide as an off-white solid.

### Step 3

Proceeding as described in Example 1 above, but using 2-(3-bromophenyl)propanoyl azide and 4-(2-methylpyridin-4-yl)benzenamine gave the product 1-(1-(2-bromophenyl)-3-methylbutyl)-3-(4-(2-methylpyridin-4-yl)phenyl)urea as a light-yellow solid. MS (ESI pos. ion) m/z: 452 (M+1).

### Example 144

### Synthesis of 1-(4-(2-methylpyridin-4-yl)phenyl)-3-(1-(4-(pyridin-3-yl)phenyl)ethyl)urea

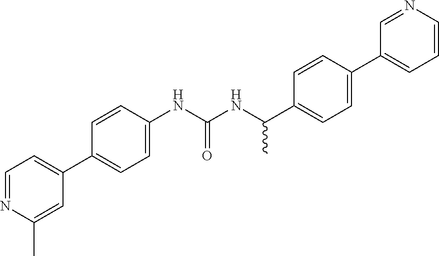

### Step 1

A 100 mL, round-bottomed flask containing a suspension of 4-(2-methylpyridin-4-yl)benzenamine (0.622 g, 3.4 mmol) in 1,2-dichloroethane (15 mL) was treated with n,n-diisopropylethylamine (0.885 mL, 5.1 mmol) followed by treatment with (+/−)-1-(4-bromophenyl)ethyl isocyanate (0.95 g, 4.2 mmol), and the reaction mixture was stirred at room temperature. Formation of a very fine precipitate was observed and the reaction was allowed to stir for 12 hours. The solvent was removed from the reaction mixture and the resulting crude product was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(1-(4-bromophenyl)ethyl)-3-(4-(2-methylpyridin-4-yl)phenyl)urea as an off-white solid. MS (ESI pos. ion) m/z: 410 (M+1).

### Step 2

A 2 mL microwave synthesizer vessel containing a suspension of 1-(1-(4-bromophenyl)ethyl)-3-(4-(2-methylpyridin-4-yl)phenyl)urea (0.338 g, 0.82 mmol), pyridine-3-boronic acid (0.233 g, 1.9 mmol), dichlorobis(di-tert-butylphenylphosphine)palladium(II) (0.015 g, 0.024 mmol) and potassium phosphate, tribasic (0.534 g, 2.5 mmol) in 1,4-dioxane/water (3.6 mL, 5/1) was heated to 130° C. for 30 minutes. The reaction mixture was concentrated in the genevac and the resulting residue was dissolved in methanol/DMSO (9.5 mL, 1/5). The insoluble solids were removed by filtration and the filtrate was concentrated resulting in a residue which was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(4-(2-methylpyridin-4-yl)phenyl)-3-(1-(4-(pyridin-3-yl)phenyl)ethyl)urea as a brown solid. MS (ESI pos. ion) m/z: 409 (M+1).

### Example 145

### Synthesis of 1-(1-(3-cyanophenyl)ethyl)-3-(4-(2-methylpyridin-4-yl)phenyl)urea

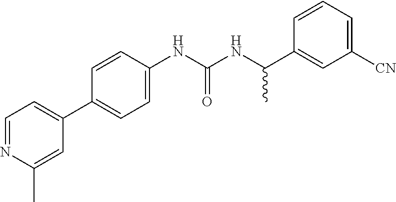

Proceeding as described in Example 141 above, but using 1-(1-(3-bromophenyl)ethyl)-3-(4-(2-methylpyridin-4-yl)phenyl)urea and zinc cyanide gave the product 1-(1-(3-cyanophenyl)ethyl)-3-(4-(2-methylpyridin-4-yl)phenyl)urea as an off-white solid. MS (ESI pos. ion) m/z: 357 (M+1).

### Example 146

### Synthesis of 1-(1-(4-cyanophenyl)ethyl)-3-(4-(2-methylpyridin-4-yl)phenyl)urea

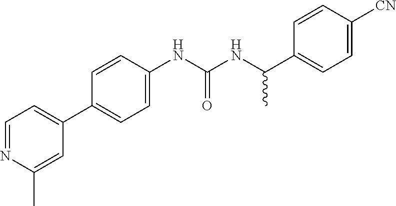

Proceeding as described in Example 141 above, but using 1-(1-(4-bromophenyl)ethyl)-3-(4-(2-methylpyridin-4-yl)phenyl)urea and zinc cyanide gave the product 1-(1-(4-cyanophenyl)ethyl)-3-(4-(2-methylpyridin-4-yl)phenyl)urea as an off-white solid. MS (ESI pos. ion) m/z: 357 (M+1).

### Example 147

### Synthesis of 1-(4-(2-methylpyridin-4-yl)phenyl)-3-(1-m-tolylethyl)urea

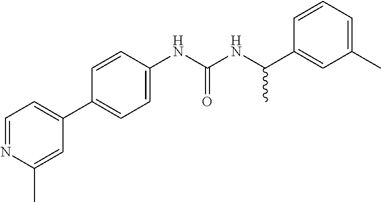

### Step 1

To a solution of methyl 2-m-tolylacetate (5.0 g, 30 mmol) in 100 mL of THF was added sodium hydride, 60% dispersion in mineral oil (0.91 mL, 37 mmol) in portions. After stirring at room temperature for 10 minutes, iodomethane (2.1 mL, 33 mmol) was added and the resulting mixture was stirred at room temperature for 12 hours. The reaction was quenched with brine, extracted with ethyl acetate, dried over sodium sulfate, filtered, and evaporated to dryness. The crude product was purified by silica gel flash column chromatography (eluted using 10%-100% EtOAc/Hexane gradient) to give the methyl 2-m-tolylpropanoate. MS (ESI, positive ion) m/z: 179 (M+1).

### Step 2

To methyl 2-m-tolylpropanoate (1.33 g, 7.462 mmol) in MeOH/THF/water (3:3:1; 14 mL) was added lithium hydroxide monohydrate (1.096 g, 26.1 mmol). After complete consumption of starting material the solvent was evaporated under high vacuum. The residue was re-dissolved in water and the solution was adjusted to pH ˜1.0, extracted with dichloromethane, dried over sodium sulfate, filtered through a silica-gel pad and evaporated to give 2-m-tolylpropanoic acid as a white solid. MS (ESI, positive ion) m/z: 165 (M+1).

### Step 3

A 100 mL, round-bottomed flask containing a suspension of 2-m-tolylpropanoic acid (0.467 g, 2.8 mmol) in THF (10 mL) at 0° C. was treated with triethylamine (1.2 ml, 8.6 mmol). The resulting mixture was stirred at 0° C. for 10 minutes. Then, ethyl chloroformate (0.435 ml, 4.6 mmol) was added and the mixture was stirred at 0° C. for an additional 60 minutes. Then, a solution of sodium azide (0.590 g, 9.1 mmol) in water (3.0 mL) was added and the mixture was stirred at room temperature for 16 hours. Then, H₂O (25 mL) was added and the mixture was extracted with EtOAc (2×50 mL). The combined organic extracts were dried over Na₂SO₄, concentrated, and dried in vacuum to give 2-m-tolylpropanoyl azide as reddish-yellow oil.

### Step 4

Proceeding as described in Example 7 above, but using 2-m-tolylpropanoyl azide and 4-(2-methylpyridin-4-yl)benzenamine gave the product 1-(4-(2-methylpyridin-4-yl)phenyl)-3-(1-m-tolylethyl)urea as an off-white solid. MS (ESI pos. ion) m/z: 346 (M+1).

### Example 148

### Synthesis of 1-(4-(2-methylpyridin-4-yl)phenyl)-3-(1-(quinolin-6-yl)ethyl)urea

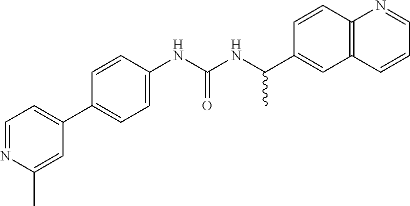

### Step 1

A mixture of 2-(4-aminophenyl)acetic acid (15.27 g, 101 mmol), Glycerol (37 g, 404 mmol), iron(II) sulfate heptahydrate (4.2 g, 15 mmol), nitrobenzene (7.3 mL, 71 mmol), and sulfuric acid concentrate (17 mL, 202 mmol) was stirred at room temperature, then was heated to 130° C. for 5 hours. The mixture was concentrated in vacuo. The crude was added 10N NaOH (90 mL). The mixture was filtered through celite. The filtrate was acidified with glacial acetic acid. The dark precipitate was collected by filtration and washed with water. The brown solid was added 1N NaOH (150 mL) and carbon (8.5 g). The mixture was stirred overnight. The solid was filtered through celite. The filtrate was acidified with glacial acetic acid. The brown precipitate was collected by filtration and washed with water and 2-(quinolin-6-yl)acetic acid was obtained as a tan solid. MS (ESI pos. ion) m/z: 188 (M+1).

### Step 2

To a solution of 2-(quinolin-6-yl)acetic acid (0.4 g, 2 mmol) in THF (10 mL), Sodium bis(trimethylsilyl)amide, 1M in THF (5 mL, 5 mmol) was added dropwise from a syringe. After 15 minutes at room temperature iodomethane, reagent plus, 99.5% stab with copper (0.175 mL, 3 mmol) was added. The mixture was stirred at room temperature for another 4 hours. The reaction mixture was quenched after another hour at room temperature, diluted with 1N HCl solution, and extracted with ethyl acetate (75 mL). The aqueous layer was concentrated in the genevac and the resulting residue was taken up in THF (20 mL). The undissolved solids were removed by filtration. The filtrate was concentrated on the rotary evaporator, resulting residue was dried under high vacuum, and obtained the product 2-(quinolin-6-yl)propanoic acid. MS (ESI pos. ion) m/z: 202 (M+1).

### Step 3

A 100 mL, round-bottomed flask containing a suspension of 2-(quinolin-6-yl)propanoic acid (0.330 g, 1.64 mmol) in THF (10 mL) at 0° C. was treated with triethylamine (0.600 mL, 4.31 mmol). The resulting mixture was stirred at 0° C. for 10 minutes. Then, ethyl chloroformate (0.205 mL, 2.15 mmol) was added and the mixture was stirred at 0° C. for an additional 60 minutes. Then, a solution of sodium azide (0.320 g, 4.92 mmol) in water (3.0 mL) was added and the mixture was stirred at room temperature for 16 hours. Then, H₂O (25 mL) was added and the mixture was extracted with EtOAc (2×50 mL). The combined organic extracts were dried over Na₂SO₄, concentrated, and dried in vacuum to give 2-(quinolin-6-yl)propanoyl azide as brown oil.

### Step 4

Proceeding as described in Example 7 above, but using 2-(quinolin-6-yl)propanoyl azide and 4-(2-methylpyridin-4-yl)benzenamine gave the product 1-(4-(2-methylpyridin-4-yl)phenyl)-3-(1-(quinolin-6-yl)ethyl)urea as a tan solid. MS (ESI pos. ion) m/z: 383 (M+1).

### Example 149

### Synthesis of 1-(1-(3-methoxyquinolin-6-yl)ethyl)-3-(4-(2-methylpyridin-4-yl)phenyl)urea

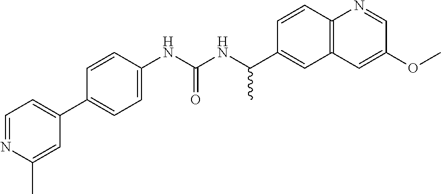

### Step 1

To a stirring solution of tert-butyl 2-(3-methoxyquinolin-6-yl)acetate (0.60 g, 2.2 mmol) in THF (10 mL) at −78° C. under nitrogen was added lithium bis(trimethylsilyl)amide, 1M in THF (2.4 mL, 2.4 mmol). Then, iodomethane, reagentplus, 99.5% stab with copper (0.145 mL, 2.3 mmol) was added after 15 minutes of stirring. The cooling bath was removed after 40 minutes. During work up the reaction was quenched with saturated ammonium chloride (5 mL), and partitioned between ethyl acetate (75 mL) and 5% sodium bicarbonate (15 mL). The organic layer was dried over anhydrous sodium sulfate, concentrated to yield tert-butyl 2-(3-methoxyquinolin-6-yl)propanoate as a brown oil. MS (ESI pos. ion) m/z: 288 (M+1).

### Step 2

A 50 mL-round bottomed flask, containing a solution of tert-butyl 2-(3-methoxyquinolin-6-yl)propanoate (0.63 g, 2 mmol) in dichloromethane (8 mL) was treated with trifluoroacetic acid (4 mL, 54 mmol) and the reaction mixture was stirred at room temperature for 6 hours. Then reaction mixture was concentrated to dryness in the genevac, dried under high vacuum to yield 2-(3-methoxyquinolin-6-yl)propanoic acid. MS (ESI pos. ion) m/z: 232.

### Step 3

A 100 mL, round-bottomed flask containing a suspension of 2-(3-methoxyquinolin-6-yl)propanoic acid (0.220 g, 0.951 mmol) in THF (10 mL) at 0° C. was treated with triethylamine (0.350 mL, 2.52 mmol). The resulting mixture was stirred at 0° C. for 10 minutes. Then, ethyl chloroformate (0.125 ml, 1.31 mmol) was added and the mixture was stirred at 0° C. for an additional 60 minutes. Then, a solution of sodium azide (0.185 g, 2.85 mmol) in water (2.0 mL) was added and the mixture was stirred at room temperature for 16 hours. Then, H₂O (25 mL) was added and the mixture was extracted with EtOAc (2×50 mL). The combined organic extracts were dried over Na₂SO₄, concentrated, and dried in vacuum to give 2-(3-methoxyquinolin-6-yl)propanoyl azide as a brown oil.

### Step 4

Proceeding as described in Example 7 above, but using 2-(3-methoxyquinolin-6-yl)propanoyl azide and 4-(2-methylpyridin-4-yl)benzenamine gave the product 1-(1-(3-methoxyquinolin-6-yl)ethyl)-3-(4-(2-methylpyridin-4-yl)phenyl)urea as an off-white solid. MS (ESI pos. ion) m/z: 413 (M+1).

### Example 150

### Synthesis of 1-(3-methyl-1-phenylbutyl)-3-(4-(2-methylpyridin-4-yl)phenyl)urea

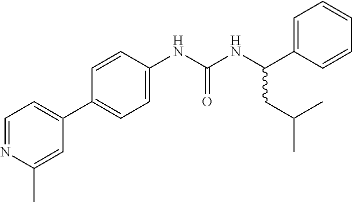

### Step 1

To a solution of 3-bromophenylacetic acid (3.00 g, 14 mmol) (azeotroped with toluene) in dry toluene (2 mL) was added sodium bis(trimethylsilyl)amide (1.0M solution in tetrahydrofuran, 32 mL, 32 mmol). After stirring at room temperature for 20 minutes, 1-iodo-2-methylpropane (2 mL, 15 mmol) was added dropwise. After 10 minutes, the reaction was quenched with 2 N HCl to pH ˜2, extracted with ethyl acetate, dried over sodium sulfate, filtered, and evaporated to dryness. The crude product was purified by silica gel flash column chromatography (eluted using 10%-100% EtOAc/Hexane gradient) to give 2-(3-bromophenyl)-4-methylpentanoic acid. MS (ESI, positive ion) m/z: 271 (M+1).

### Step 2

A 100 mL, round-bottomed flask containing a suspension of 2-(3-bromophenyl)-4-methylpentanoic acid (1.185 g, 4.37 mmol) in THF (15 mL) at 0° C. was treated with triethylamine (1.800 ml, 12.9 mmol). The resulting mixture was stirred at 0° C. for 10 minutes. Then, ethyl chloroformate (0.600 ml, 6.28 mmol) was added and the mixture was stirred at 0° C. for an additional 60 minutes. Then, a solution of sodium azide (0.955 g, 14.7 mmol) in water (3.5 mL) was added and the mixture was stirred at room temperature for 16 hours. Then, H₂O (25 mL) was added and the mixture was extracted with EtOAc (75 mL). The combined organic extracts were dried over Na₂SO₄, concentrated, and dried in vacuum to give 2-(3-bromophenyl)-4-methylpentanoyl azide as brown oil.

### Step 3

Proceeding as described in Example 7 above, but using 2-(3-bromophenyl)-4-methylpentanoyl azide and 4-(2-methylpyridin-4-yl)benzenamine gave the product 1-(1-(3-bromophenyl)-3-methylbutyl)-3-(4-(2-methylpyridin-4-yl)phenyl)urea. MS (ESI pos. ion) m/z: 452 (M+1).

### Step 4

A solution of 1-(1-(3-bromophenyl)-3-methylbutyl)-3-(4-(2-methylpyridin-4-yl)phenyl)urea (0.345 g, 0.763 mmol) in ethanol (10 mL) was stirred with palladium hydroxide, 20 wt % pd (dry basis) on carbon, wet, degussa type e101 ne/w (0.165 g, 1.17 mmol) under hydrogen atmosphere (balloon) at room temperature for additional 17 hours at room temperature. The catalyst was removed by filtration over a celite-pad, washed with ethanol (10 mL). The combined filtrates were concentrated to yield the crude product which was purified by preparative HPLC [gradient 10-90% MeCN (0.1% TFA)/H₂O (0.1% TFA)] to give the pure product which was dissolved in methanol (5 mL) and neutralized by passing the solution through a Polymer Lab-HCO₃ macroporous resin cartridge, and the filtrate was concentrated to give 1-(3-methyl-1-phenylbutyl)-3-(4-(2-methylpyridin-4-yl)phenyl)urea as an off-white solid. MS (ESI pos. ion) m/z: 374 (M+1).

### Example 151

### Synthesis of 1-(2,2-difluoro-1-phenylethyl)-3-(4-(2-methylpyridin-4-yl)phenyl)urea

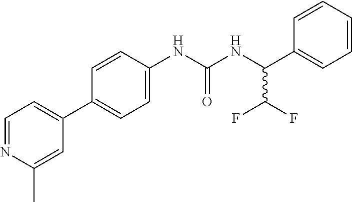

### Step 1

A mixture of 2,2-difluoro-1-phenylethanone (0.573 g, 3.7 mmol), ammonium chloride (0.402 g, 7.5 mmol), titanium (IV) isopropoxide (2.2 mL, 7.5 mmol), and triethylamine (1.050 mL, 7.5 mmol) in absolute ethanol (9 mL) was stirred at room temperature (formation of a milky white precipitate was observed) for 8 hours. Then, sodium borohydride (0.375 g, 9.9 mmol) was added and the resulting mixture was stirred for additional 12 hours. The white precipitate (Titanium Salts) was removed by filtration, washed with ethanol (25 mL). The combined filtrates were concentrated to yield a gummy white solid, which was taken up in ethyl acetate (75 mL) and water (30 mL). Formation of more white precipitate (Titanium Salts) was seen. The mixture was stirred vigorously for an hour. Celite was added to this mixture and the solids were removed by filtration, the filter cake was washed with ethyl acetate (35 mL). The filtrate was transferred to a separatory funnel, ethyl acetate layer was separated, dried over anhydrous sodium sulfate, and concentrated to give 2,2-difluoro-1-phenylethanamine as a colorless oil. MS (ESI pos. ion) m/z: 158 (M+1).

### Step 2

Proceeding as described in Example 7 above, but using 4-(2-methylpyridin-4-yl)benzoyl azide and 2,2-difluoro-1-phenylethanamine gave the product 1-(2,2-difluoro-1-phenylethyl)-3-(4-(2-methylpyridin-4-yl)phenyl)urea as an off-white solid. MS (ESI pos. ion) m/z: 368 (M+1).

### Example 152

### Synthesis of 1-(1-(isoquinolin-6-yl)ethyl)-3-(4-(2-methylpyridin-4-yl)phenyl)urea

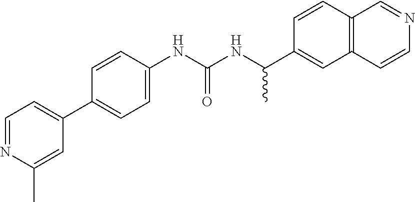

### Step 1

To a stirring solution of tert-butyl 2-(isoquinolin-6-yl)acetate (0.830 g, 3.4 mmol) in THF (7.0 mL) at −75° C. under nitrogen was added Lithium bis(trimethylsilyl)amide, 1M in THF (3.8 mL, 3.8 mmol). Then, iodomethane, 99%, stabilized with copper (0.23 mL, 3.7 mmol) was added after 15 minutes of stirring. The cooling bath was removed after 40 minutes. During work up the reaction was quenched with saturated ammonium chloride (5 mL), and partitioned between ethyl acetate (75 mL) and 5% sodium bicarbonate (15 mL). The organic layer was dried over anhydrous sodium sulfate, concentrated to yield the crude product which was purified by silica gel flash column chromatography (eluted using 10%-100% EtOAc/Hexane gradient) to give tert-butyl 2-(isoquinolin-6-yl)propanoate. MS (ESI, positive ion) m/z: 258 (M+1).

### Step 2

A 50 mL-round bottomed flask, containing a solution of tert-butyl 2-(isoquinolin-6-yl)propanoate (0.95 g, 3.7 mmol) in dichloromethane (10 mL) was treated with trifluoroacetic acid (5.0 mL, 67 mmol) and the reaction mixture was stirred at room temperature for 16 hours. Then the reaction mixture was concentrated to dryness in the genevac, dried under high vacuum to yield 2-(isoquinolin-6-yl)propanoic acid. MS (ESI pos. ion) m/z: 202.

### Step 3

A 100 mL, round-bottomed flask containing a suspension of 2-(isoquinolin-6-yl)propanoic acid (0.690 g, 2.19 mmol) in THF (8 mL) at 0° C. was treated with triethylamine (1.00 ml, 7.19 mmol). The resulting mixture was stirred at 0° C. for 10 minutes. Then, ethyl chloroformate (0.475 ml, 4.97 mmol) was added and the mixture was stirred at 0° C. for an additional 60 minutes. Then, a solution of sodium azide (0.460 g, 7.08 mmol) in water (1.5 mL) was added and the mixture was stirred at room temperature for 16 hours. Then, H₂O (25 mL) was added and the mixture was extracted with EtOAc (75 mL). The combined organic extracts were dried over Na₂SO₄, concentrated, and dried in vacuum to give 2-(isoquinolin-6-yl)propanoyl azide as a brown oil.

### Step 4

Proceeding as described in Example 7 above, but using 2-(isoquinolin-6-yl)propanoyl azide and 4-(2-methylpyridin-4-yl)benzenamine gave the product 1-(1-(isoquinolin-6-yl)ethyl)-3-(4-(2-methylpyridin-4-yl)phenyl)urea as light-yellow solid. MS (ESI pos. ion) m/z: 383 (M+1).

### Example 153

### Synthesis of 1-(1-(3,5-bis(trifluoromethyl)phenyl)ethyl)-3-(4-(2-methylpyridin-4-yl)phenyl)urea

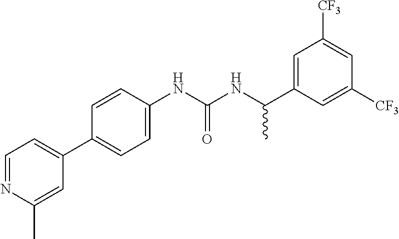

A mixture of 4-(2-methylpyridin-4-yl)benzenamine (72 mg, 0.389 mmol), N,N′-disuccinimidyl carbonate (149 g, 0.583 mmol) and DMF (2 mL) was stirred at room temperature overnight. Then 1-(3,5-bis(trifluoromethyl)phenyl)ethanamine (200 g, 0.778 mmol) and N-ethyl-N-isopropylpropan-2-amine (0.27 mL, 1.56 mmol) was added. The mixture was stirred overnight, diluted with Na₂CO₃ and extracted with EtOAc. The organic layer was dried over Na₂SO₄ and concentrated in-vacuo. The crude was purified by silica gel chromatography using EtOAc-CH₂Cl₂ as the eluant to yield 1-(1-(3,5-bis(trifluoromethyl)-phenyl)ethyl)-3-(4-(2-methylpyridin-4-yl)phenyl)urea as a white solid. MS (ESI pos. ion) m/z: 468 (M+1).

### Example 154

### Synthesis of 1-(4-ethoxy-5-isopropyl-2-methylphenyl)-3-(4-(2-methylpyridin-4-yl)phenyl)urea

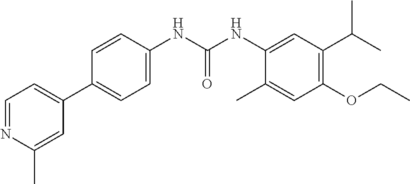

### Step 1

To a solution of 4-(2-methylpyridin-4-yl)benzenamine (0.202 g, 1.10 mmol) in CH₂Cl₂ (10 mL) was added 1,1′-carbonyldiimidazole (0.189 g, 1.17 mmol) as a solid at rt. After 4 h, 4-ethoxy-5-isopropyl-2-methyl-phenylamine hydrochloride (0.249 g, 1.08 mmol; Matrix Scientific) was added to the reaction followed by triethylamine (0.650 mL, 4.67 mmol). The reaction was stirred overnight and then concentrated to dryness. The residue was dissolved in DMSO/MeOH and purified by reverse-phase HPLC (Gilson; XTerra Prep RP18 OBD™, 19×150 mm column) eluting with 0.1% TFA-H₂O:0.1% TFA CH₃CN (9:1→1:9). The fractions containing the desired product were combined and concentrated in vacuo. The residue was dissolved in MeOH and loaded onto an SCX II cartridge eluting with MeOH then 2M NH₃ in MeOH to give 1-(4-ethoxy-5-isopropyl-2-methylphenyl)-3-(4-(2-methylpyridin-4-yl)phenyl)urea as a white amorphous solid. MS (ESI pos. ion) m/z: 404 (M+1).

### Example 155

### Synthesis of 1-(4-(2-methylpyridin-4-yl)phenyl)-3-(4-phenylthiazol-2-yl)urea

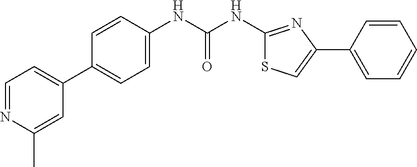

### Step 1

To a solution of 2-amino-4-phenylthiazole (0.200 g, 1.13 mmol; Oakwood Products) in CH₂Cl₂ (10 mL) was added 1,1′-carbonyldiimidazole (0.193 g, 1.19 mmol) as a solid at room temperature. After 4 h, 4-(2-methylpyridin-4-yl)benzenamine (0.210 g, 1.14 mmol) was added to the reaction followed by triethylamine (0.500 ml, 3.59 mmol). After stirring overnight the reaction mixture was filtered and the solid was washed with CH₂Cl₂ and dried in vacuo to give 1-(4-(2-methylpyridin-4-yl)phenyl)-3-(4-phenylthiazol-2-yl)urea as a white amorphous solid. MS (ESI pos. ion) m/z: 387 (M+1).

### Example 156

### Synthesis of 1-(4-(2-methylpyridin-4-yl)phenyl)-3-(1-phenylcyclopropyl)urea

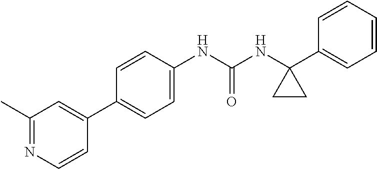

A solution of azido(1-phenylcyclopropyl)methanone (300 mg, 1.6 mmol) in THF (1.5 mL) was subjected to microwave irradiation at 120° C. for 10 min. 4-(2-Methylpyridin-4-yl)benzenamine (207 mg, 1.1 mmol) was added and the reaction mixture was subjected to a microwave irradiation at 120° C. for 15 min. Formation of yellow precipitate was observed. The precipitate was collected by filtration, washed with MeOH, and dried in vacuum to give the title compound as a yellow solid. MS (ESI, positive ion) m/z: 344 (M+1).

### Example 157

### Synthesis of 1-(4-(2-cethylpyridin-4-yl)phenyl)-3-(1-phenylcyclobutyl)urea

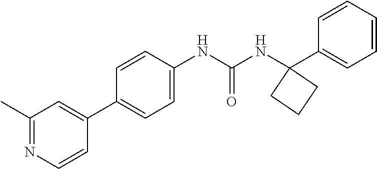

To a solution of azido(4-(2-methylpyridin-4-yl)phenyl)methanone (500 mg, 2.1 mmol) in THF (3 mL) was subjected to a microwave irradiation at 120° C. for 10 min. N,N-Diisopropylethylamine (0.548 mL, 3.148 mmol, Aldrich) and 1-phenylcyclobutanamine hydrochloride (385 mg, 2.1 mmol) were added and the reaction mixture was then subjected to a microwave irradiation at 120° C. for 15 min. Solvent was removed and MeOH (5 mL) was added to give light yellow precipitates. The precipitates was collected by filtration, washed with MeOH, and dried in vacuum to give the title compound as a light yellow solid. MS (ESI, positive ion) m/z: 358 (M+1).

Following the above procedure but substituting 1-phenylcyclobutanamine with 1-(4-chlorophenyl)cyclobutanamine gave1-(1-(4-chlorophenyl)cyclobutyl)-3-(4-(2-methylpyridin-4-yl)phenyl)urea.

### Example 158

### Synthesis of 1-(4-(2-methylpyridin-4-yl)phenyl)-3-((1-phenylcyclopentyl)methyl)urea

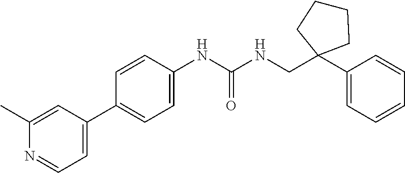

### Step 1

To a solution of 1-phenyl-1-cyclopentanecarbonitrile (1.500 mL, 8.76 mmol, Acros Organics USA) in MeOH (40 mL) was added a solution of palladium, 10 wt. % on activated carbon (93 mg, 0.876 mmol, Aldrich) in EtOAc (0.5 mL) and concentrated HCl (0.4 mL). The reaction mixture was stirred at room temperature under H₂ (42 psi) overnight. The reaction mixture was filtered through celite, washed the filter-cake with MeOH. The combined filtrates were concentrated and H₂O was added to the residue. The aqueous solution was extracted with EtOAc. The aqueous layer was basified (pH>10) using saturated aqueous NaHCO₃ and extracted with EtOAc. The combined organic extracts were dried over MgSO₄, concentrated, and dried in vacuum to give (1-phenylcyclopentyl)methanamine as light yellow oil, which was used in the next step without purification.

### Step 2

A solution of azido(4-(2-methylpyridin-4-yl)phenyl)methanone (200 mg, 0.84 mmol) in THF (0.8 mL) was subjected to a microwave irradiation at 120° C. for 10 min. Then, (1-phenylcyclopentyl)methanamine (191 mg, 1.1 mmol) was added and the reaction mixture was stirred at room temperature for overnight. Solvent was removed and the residue was mixed silica gel. The solid mixture was then purified by silica gel flash column chromatography (eluted using 10%-100% EtOAc/hexane gradient) to give the title compound as a light yellow solid. MS (ESI, positive ion) m/z: 386 (M+1).

Proceeding as described in Example 158 above, using azido(4-(2-methylpyridin-4-yl)phenyl)methanone and the corresponding amine, the following compounds were prepared.

| | | | MS: ESI |
|---|---|---|---|
| | | | m/z |
| Examples | Structure | Name | (M + 1) |
| 159 | 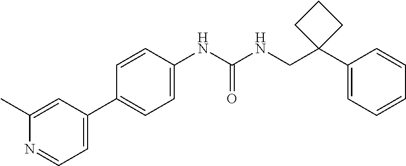 | 1-(4-(2-methylpyridin-4- yl)phenyl)-3-((1- phenylcyclobutyl)methyl)urea | 372 |
| 160 | 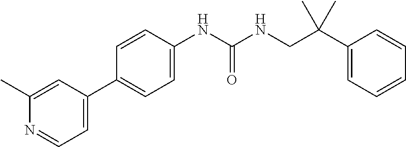 | 1-(2-methyl-2-phenylpropyl)-3- (4-(2-methylpyridin-4- yl)phenyl)urea | 360 |
| 161 | 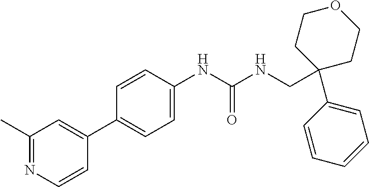 | 1-(4-(2-methylpyridin-4- yl)phenyl)-3-((4- phenyltetrahydro-2H-pyran-4- yl)methyl)urea | 402 |
| 162 | 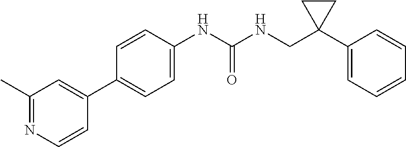 | 1-(4-(2-methylpyridin-4- yl)phenyl)-3-((1- phenylcyclopropyl)methyl)urea | 358 |

### Example 163

### Synthesis of 1-(2-methyl-1-phenylpropan-2-yl)-3-(4-(2-methylpyridin-4-yl)phenyl)urea

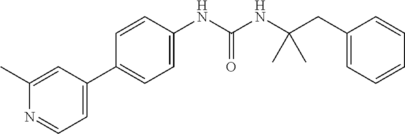

### Step 1

To a solution of ethyl 2,2-dimethyl-3-phenylpropanoate (2.0 g, 9.7 mmol) in MeOH (32 mL) at 0° C. was added a solution of lithium hydroxide monohydrate (1.2 g, 29.1 mmol) in H₂O (10 mL). After addition, the mixture was stirred at room temperature for overnight. Then, the solvent was removed and the H₂O (50 mL) was added. The aqueous mixture was adjusted to pH ˜2.0 by using concentrated HCl. Then, the aqueous was extracted with EtOAc (2×50 mL). The combined organic extracts were dried over MgSO₄, concentrated, and dried in vacuo to give 2,2-dimethyl-3-phenylpropanoic acid as a colorless oil. MS (ESI, positive ion) m/z: 177 (M+1).

### Step 2

A solution of 2,2-dimethyl-3-phenylpropanoic acid (1.47 g, 8.29 mmol) and triethylamine (1.4 mL, 9.95 mmol) in THF (40 mL) at 0° C. was added ethyl chloroformate (0.95 mL, 9.95 mmol). After addition, the mixture was then stirred at room temperature for 3 h. Then, a solution of sodium azide (0.87 mL, 24.9 mmol) in H₂O (7 mL) was added and the mixture was stirred at room temperature for 48 h. Then, H₂O (10 mL) was added and the mixture was extracted with EtOAc (2×80 mL). The combined organic extracts were dried over MgSO₄, concentrated, and dried in vacuo to give 2,2-dimethyl-3-phenylpropanoyl azide as a light yellow solid, which was used without purification.

### Step 3

A solution of 2,2-dimethyl-3-phenylpropanoyl azide (0.44 g, 2.171 mmol) in THF (0.8 mL) was subjected to a microwave irradiation at 120° C. for 10 minutes. Then, 4-(2-methylpyridin-4-yl)benzenamine (0.20 g, 1.086 mmol) was added and the mixture was subjected to a microwave irradiation at 120° C. for 15 minutes. Then, the solvent was removed and the residue was mixed with silica gel. The solid mixture was then purified by silica gel flash column chromatography using ISCO instrument (solid loading, 15%-100% EtOAc/hexane) to give the title compound, which was repurified by preparative HPLC (0%-100% MeCN 0.1% TFA/H₂O 0.1% TFA) to give a desired product in a solution of MeCN 0.1% TFA and H₂O 0.1% TFA. The solvent was removed and the aqueous solution was neutralized using NaHCO₃(s). The aqueous solution was extracted with EtOAc (2×40 mL). The combined organic extracts were then dried over MgSO₄, concentrated, and dried in vacuo to give 1-(2-methyl-1-phenylpropan-2-yl)-3-(4-(2-methylpyridin-4-yl)phenyl)urea as a light yellow solid. MS (ESI, positive ion) m/z: 360 (M+1).

### Example 164

### Synthesis of 1-(3-(4-chlorophenyl)tetrahydrofuran-3-yl)-3-(4-(2-methylpyridin-4-yl)phenyl)urea

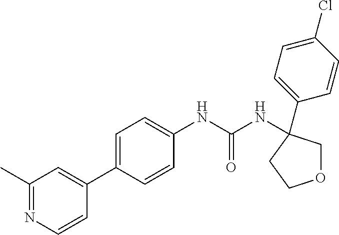

### Step 1

A solution of 3-(4-chlorophenyl)-tetrahydrofuran-3-carboxylic acid (2.898 g, 12.786 mmol) and triethylamine (1.956 mL, 14.064 mmol) in THF (42 mL) at 0° C. was added ethyl chloroformate (1.3 mL, 14.064 mmol). After addition, the mixture was stirred at room temperature for 4 h. Then, a solution of sodium azide (1.7 g, 25.5 mmol) in H₂O (5 mL) was added and the mixture was stirred at room temperature for 48 h. Then, H₂O (10 mL) was added and the mixture was extracted with EtOAc (2×60 mL). The combined organic extracts were dried over MgSO₄, concentrated, and dried in vacuo to give 3-(4-chlorophenyl)-tetrahydrofuran-3-carbonyl azide as an orange oil which was used without purification.

### Step 2

A solution of azido(3-(4-chlorophenyl)-tetrahydrofuran-3-yl)methanone (0.820 g, 3.26 mmol) in THF (1.4 mL) was subjected to a microwave irradiation at 120° C. for 15 minutes. Then, 4-(2-methylpyridin-4-yl)benzenamine (0.200 g, 1.09 mmol) was added and the mixture was subjected to a microwave irradiation at 120° C. for 15 minutes. Then, the solvent was removed and the residue was mixed with silica gel. The solid mixture was then purified by silica gel column chromatography using ISCO instrument (solid loading, 15%-100% EtOAc/hexane) to give 1-(3-(4-chlorophenyl)tetrahydrofuran-3-yl)-3-(4-(2-methylpyridin-4-yl)phenyl)urea as a light yellow solid. MS (ESI, positive ion) m/z: 408 (M+1).

### Example 165

### Synthesis of 1-(1-(4-bromophenyl)cyclobutyl)-3-(4-(2-methylpyridin-4-yl)phenyl)urea

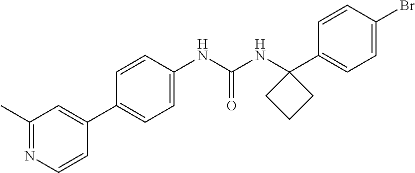

### Step 1

A solution of 1-(4-bromophenyl)cyclobutanecarboxylic acid (4.97 g, 19.5 mmol) and triethylamine (3.0 mL, 21.5 mmol) in THF (65 mL) at 0° C. was added ethyl chloroformate (2.0 mL, 21.5 mmol). After addition, the mixture was stirred at room temperature for 4 h. Then, a solution of sodium azide (2.5 g, 39.0 mmol) in H₂O (5 mL) was added and the mixture was stirred at room temperature for 48 h. Then, H₂O (10 mL) was added and the mixture was extracted with EtOAc (2×60 mL). The combined organic extracts were dried over MgSO₄, concentrated, and dried in vacuo to give azido(1-(4-bromophenyl)cyclobutyl)methanone as a yellow solid which was used without purification.

### Step 2

A solution of azido(1-(4-bromophenyl)cyclobutyl)methanone (0.61 g, 2.2 mmol) in THF (0.8 mL) was subjected to a microwave irradiation at 120° C. for 15 minutes. Then, 4-(2-methylpyridin-4-yl)benzenamine (0.200 g, 1.1 mmol) was added and the mixture was subjected to a microwave irradiation at 120° C. for 15 minutes. Then, the solvent was removed and MeOH (5 mL) was added to the residue. A light yellow precipitation was observed. The mixture was filtered and the filtrate was concentrated to give the crude product. The light yellow solid was then mixed silica gel and the solid mixture was purified by silica gel column chromatography using ISCO (solid loading, 15%-100% EtOAc/hexane) to give 1-(1-(4-bromophenyl)cyclobutyl)-3-(4-(2-methylpyridin-4-yl)phenyl)urea as an off-white solid. MS (ESI, positive ion) m/z: 436 (M+1).

Proceeding as described in Example 165 above but using 4-(2-methylpyridin-4-yl)benzenamine and the corresponding amine and the corresponding azido carbonyl intermediate, the following compounds were prepared.

| | | | MS: ESI |
|---|---|---|---|
| | | | m/z |
| Examples | Structure | Name | (M + 1) |
| 166 | 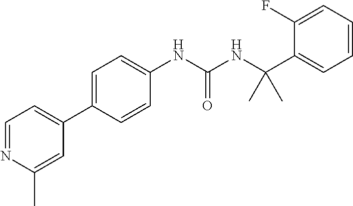 | 1-(2-(2-fluorophenyl)propan-2- yl)-3-(4-(2-methylpyridin-4- yl)phenyl)urea | 364 |
| 167 | 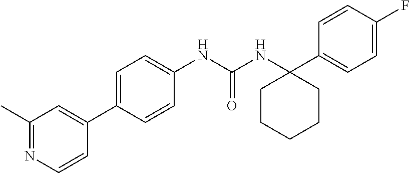 | 1-(1-(4- fluorophenyl)cyclohexyl)-3-(4- (2-methylpyridin-4- yl)phenyl)urea | 404 |
| 168 | 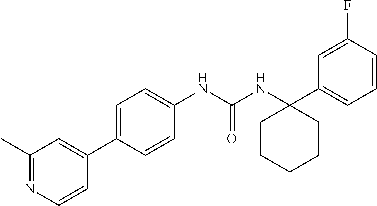 | 1-(1-(3- fluorophenyl)cyclohexyl)-3-(4- (2-methylpyridin-4- yl)phenyl)urea | 404 |
| 169 | 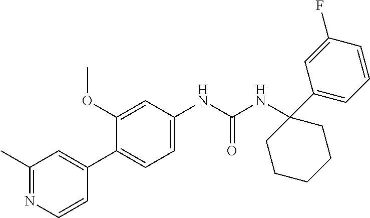 | 1-(1-(3- fluorophenyl)cyclohexyl)-3-(3- methoxy-4-(2-methylpyridin-4- yl)phenyl)urea | 434 |
| 170 | 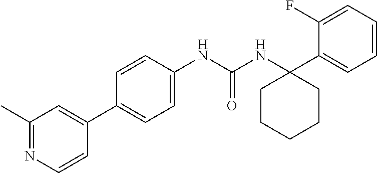 | 1-(1-(2- fluorophenyl)cyclohexyl)-3-(4- (2-methylpyridin-4- yl)phenyl)urea | 404 |

### Example 171

### Synthesis of 1-((1-(6-chloropyridin-2-yl)cyclopentyl)methyl)-3-(4-(2-methylpyridin-4-yl)phenyl)urea

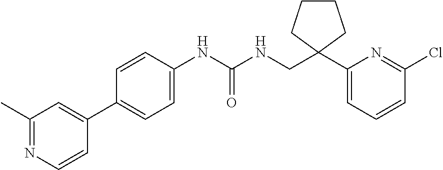

### Step 1

A solution of cyclopentanecarbonitrile (0.7 g, 0.8 ml, 8 mmol) and 2-chloro-6-fluoropyridine (1.0 g, 8 mmol) in toluene (25 mL) at 0° C. was added sodium bis(trimethylsilyl)amide (8 mL, 8 mmol) 1M in THF dropwise. After addition, the mixture was stirred at 0° C. for 1 h and 3 h at room temperature. Then, H₂O (20 mL) was added and the mixture was stirred at room temperature for 15 minutes. Then, the mixture was extracted with EtOAC (2×20 mL) and the combined organic extracts were dried over MgSO₄ and concentrated. The residue was mixed with silica gel and the solid mixture was then purified by silica gel column chromatography using ISCO instrument (solid loading, 0%-30% EtOAc/hexane) to give 1-(6-chloropyridin-2-yl)cyclopentanecarbonitrile as a colorless oil. MS (ESI, positive ion) m/z: 207 (M+1).

### Step 2

To a solution of 1-(6-chloropyridin-2-yl)cyclopentanecarbonitrile (0.456 g, 2.21 mmol) in THF (3 mL) was added 1.0 M Borane THF complex (11.0 ml, 11.0 mmol) dropwise. After addition, the mixture was refluxed for 20 minutes. Then, the mixture was cooled to 0° C. and concentrated HCl (11 mL) was added dropwise. The mixture was then stirred at room temperature for 20 minutes. Then, the mixture was adjusted to pH ˜14 by using NaOH 10N at 0° C. Then, EtOAc (150 mL) was added and the mixture was stirred at room temperature for 15 minutes. The organic layer was collected, dried over MgSO₄, and concentrated. The residue was mixed with silica gel and the solid mixture was purified by silica gel column chromatography using ISCO instrument (solid loading, 0%-20% MeOH/DCM) to give 1-(6-chloropyridin-2-yl)cyclopentyl)methanamine as a colorless oil. MS (ESI, positive ion) m/z: 211 (M+1).

### Step 3

A solution of azido(4-(2-methylpyridin-4-yl)phenyl)methanone (0.130 g, 0.546 mmol) in THF (0.8 mL) was subjected to a microwave irradiation at 120° C. for 15 minutes. Then, (1-(6-chloropyridin-2-yl)cyclopentyl)methanamine (0.126 g, 0.600 mmol) was added and the mixture was stirred at room temperature for overnight. Then, the solvent was removed and the residue was mixed with silica gel. The solid mixture was then purified by column chromatography using ISCO instrument (solid loading, 15%-100% EtOAc/hexane) to give 1-((1-(6-chloropyridin-2-yl)cyclopentyl)methyl)-3-(4-(2-methylpyridin-4-yl)phenyl)urea as a light yellow solid. MS (ESI, positive ion) m/z: 421 (M+1).

### Example 172

### Synthesis of 1-(4-(2-methylpyridin-4-yl)phenyl)-3-((1-(pyridin-2-yl)cyclopentyl)methyl)urea

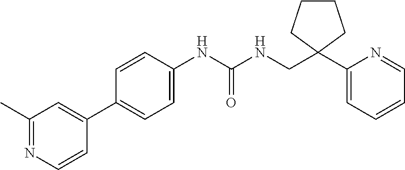

### Step 1

A solution of 1-((1-(6-chloropyridin-2-yl)cyclopentyl)methyl)-3-(4-(2-methylpyridin-4-yl)phenyl)urea (0.096 g, 0.2 mmol) in MeOH (1 mL) was stirred at room temperature under H₂ (45 psi) for 12 h. Then, palladium hydroxide, 20 wt % pd (dry basis) on carbon, wet, degussa type e101 ne/w (0.02 g, 0.02 mmol) was added and the mixture was continued stirring at room temperature under H₂ (45 psi) for another 12 h. The mixture was filtered through celite-pad, washed with MeOH (2×20 mL). The combined filtrates were concentrated and the residue was purified by silica gel chromatography using ISCO instrument (solid loading, 15%-100% EtOAc/hexane) to give 1-(4-(2-methylpyridin-4-yl)phenyl)-3-((1-(pyridin-2-yl)cyclopentyl)methyl)urea as a white solid. MS (ESI, positive ion) m/z: 387 (M+1).

### Example 173

### Synthesis of 1-(1-(3-bromophenyl)cyclobutyl)-3-(4-(2-methylpyridin-4-yl)phenyl)urea

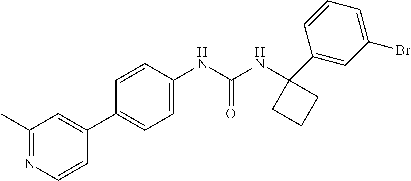

### Step 1

To a solution of 3-bromobenzeneacetonitrile (6.480 g, 33.054 mmol), potassium hydroxide (14.8 g, 26.4 mmol), and tetrabutylammonium bromide (0.107 g, 0.331 mmol) in toluene (90 mL) and H₂O (4 mL) was added 1,3-dibromopropane (3.4 mL, 33.0 mmol). The resulting mixture was then refluxed for 1.5 h. Then, the mixture was cooled to room temperature and H₂O (100 mL) was added. The mixture was then extracted with EtOAc (2×100 mL). The combined organic extracts were dried over MgSO₄ and concentrated. The residue was then mixed with silica gel and the solid mixture was purified by silica gel flash column chromatography using ISCO instrument (solid loading, 0%-100% EtOAc/hexane) to give 1-(3-bromophenyl)cyclobutanecarbonitrile as a yellow oil. MS (ESI, positive ion) m/z: 236 (M+1).

### Step 2

A solution of 1-(3-bromophenyl)cyclobutanecarbonitrile (1.520 g, 6.4 mmol) in EtOH (25 mL) was added potassium hydroxide (0.433 g, 7.725 mmol). The resulting mixture was then refluxed for overnight. Then, the mixture was cooled to 0° C. and H₂O (30 mL) was added. Then, the mixture was adjusted to pH ˜2.0 at 0° C. by using concentrated HCl. Then, EtOAc (50 mL) was added and the mixture was stirred at room temperature for 15 minutes. The organic layer was collected and the aqueous layer was extracted with EtOAc (2×50 mL). The combined organic extracts were dried over MgSO₄ and concentrated. The residue was then mixed silica gel and the solid mixture was then purified by silica gel column chromatography using ISCO (0%-15% MeOH/DCM) to give 1-(3-bromophenyl)cyclobutanecarboxylic acid as a white solid. MS (ESI, positive ion) m/z: 255 (M+1).

### Step 3

A solution of 1-(3-bromophenyl)cyclobutanecarboxylic acid (0.487 g, 1.9 mmol) and triethylamine (0.319 mL, 2.291 mmol) in THF (13 mL) at 0° C. was added ethyl chloroformate (0.219 mL, 2.291 mmol). After addition, the mixture was stirred at room temperature for 2 h. Then, a solution of sodium azide (0.5 g, 7.636 mmol) in H₂O (2 mL) was added and the mixture was stirred at room temperature for 48 h. Then, H₂O (10 mL) was added and the mixture was extracted with EtOAc (2×50 mL). The combined organic extracts were dried over MgSO₄, concentrated, and dried in vacuo to give 1-(3-bromophenyl)cyclobutanecarbonyl azide as a light yellow liquid which was used without purification.

### Step 4

A solution of azido(1-(3-bromophenyl)cyclobutyl)methanone (0.334 g, 1.19 mmol) in THF (1 mL) was subjected to a microwave irradiation at 120° C. for 15 minutes. Then, 4-(2-methylpyridin-4-yl)benzenamine (0.100 g, 0.543 mmol) was added and the mixture was subjected to microwave irradiation at 120° C. for 15 minutes. Then, the solvent (THF) was removed and a solution of DMSO and MeOH (1:1, 1 mL) was added. The solution mixture was then purified preparative HPLC (0%-100% MeCN 0.1% TFA/H₂O 0.1% TFA) to give the title compound in a solution of MeCN 0.1% TFA and H₂O 0.1% TFA. The solvent was removed and the aqueous solution was neutralized using NaHCO₃(s). The aqueous solution was extracted with EtOAc (2×20 mL). The combined organic extracts were then dried over MgSO₄, concentrated, and dried in vacuo to give 1-(1-(3-bromophenyl)cyclobutyl)-3-(4-(2-methylpyridin-4-yl)phenyl)urea as a light yellow solid. MS (ESI, positive ion) m/z: 436 (M+1).

### Example 174

### Synthesis of 1-((1-(2-chloropyridin-4-yl)cyclopentyl)methyl)-3-(4-(2-methylpyridin-4-yl)phenyl)urea

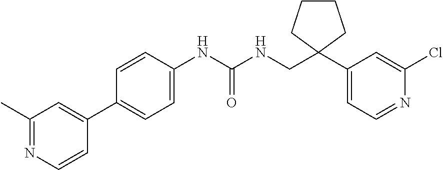

### Step 1

To a solution of cyclopentanecarbonitrile (2.36 ml, 22.6 mmol) and 2-chloro-4-fluoropyridine (2.97 g, 22.6 mmol) in toluene (60 mL) at 0° C. was added sodium bis(trimethylsilyl)amide 1.0 M in THF (22.6 mL, 22.6 mmol) dropwise. After addition, the mixture was stirred at 0° C. for 1 h and 3 h at room temperature. Then, H₂O (60 mL) was added and the mixture was stirred at room temperature for 15 minutes. Then, the mixture was extracted with EtOAc (2×20 mL) and the combined organic extracts were dried over MgSO₄ and concentrated. The residue was mixed with silica gel and the solid mixture was then purified by silica gel column chromatography using ISCO instrument (solid loading, 0%-30% EtOAc) to give 1-(2-chloropyridin-4-yl)cyclopentanecarbonitrile as a colorless liquid. MS (ESI, positive ion) m/z: 207 (M+1).

### Step 2

To a solution of 1-(2-chloropyridin-4-yl)cyclopentanecarbonitrile (1.00 g, 4.84 mmol) in toluene (6 mL) was added Boran-THF complex (0.416 g, 4.84 mmol) 1.0M in THF dropwise. After addition, the mixture was refluxed for 20 minutes. Then, the mixture was cooled to 0° C. and concentrated HCl (24 mL) was added. The mixture was then stirred at room temperature for 20 minutes. Then, the mixture was adjusted to pH ˜14 by NaOH 10N at 0° C. Then, EtOAc (150 mL) was added and the mixture was stirred at room temperature for 15 minutes. The organic layer was collected, dried over MgSO₄, and concentrated. The residue was mixed with silica gel and the solid mixture was purified by silica gel column chromatography using ISCO instrument (solid loading, 0%-20% MeOH/DCM) to give 1-(2-chloropyridin-4-yl)cyclopentyl)methanamine as a light yellow oil. MS (ESI, positive ion) m/z: 211 (M+1).

### Step 3

A solution of azido(4-(2-methylpyridin-4-yl)phenyl)methanone (0.200 g, 0.839 mmol) in THF (0.6 mL) was subjected to a microwave irradiation at 120° C. for 15 minutes. Then, (1-(2-chloropyridin-4-yl)cyclopentyl)methanamine (0.195 g, 0.923 mmol) was added and the mixture was subjected to a microwave irradiation at 120° C. for 15 minutes. Then, the mixture was filtered and the filtrate was purified by preparative HPLC (0%-100% MeCN 0.1% TFA/H₂O 0.1% TFA) to give a desired product in solution of MeCN 0.1% TFA/H₂O 0.1% TFA. The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-((1-(2-chloropyridin-4-yl)cyclopentyl)methyl)-3-(4-(2-methylpyridin-4-yl)phenyl)urea as a light yellow solid. MS (ESI, positive ion) m/z: 421 (M+1).

### Example 175

### Synthesis of 1-(4-(2-methylpyridin-4-yl)phenyl)-3-((1-(pyridin-4-yl)cyclopentyl)methyl)urea

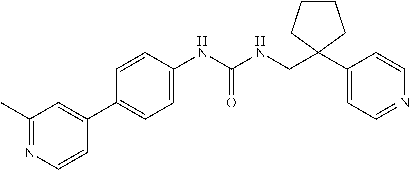

To a solution of 141-(2-chloropyridin-4-yl)cyclopentyl)methyl)-3-(4-(2-methylpyridin-4-yl)phenyl)urea (0.085 g, 0.20 mmol) in MeOH (1 mL) was added palladium hydroxide, 20 wt % pd (dry basis) on carbon, wet (0.0043 g, 0.0061 mmol). The resulting mixture was then stirred at room temperature under H₂ (42 psi) for overnight. Then, the mixture was filtered through celite-pad and washed with MeOH (2×5 mL). The combined filtrates were concentrated and the residue was mixed with silica gel. The solid mixture was purified by silica gel column chromatography using ISCO instrument (solid loading, 15%-100% EtOAc/hexane) to give 1-(4-(2-methylpyridin-4-yl)phenyl)-3-((1-(pyridin-4-yl)cyclopentyl)methyl)urea as a light yellow solid. MS (ESI, positive ion) m/z: 387 (M+1).

### Example 176

### Synthesis of 1-((1-(2-cyanopyridin-4-yl)cyclopentyl)methyl)-3-(4-(2-methylpyridin-4-yl)phenyl)urea

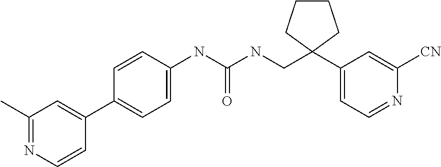

### Step 1

A solution of 1-((1-(2-chloropyridin-4-yl)cyclopentyl)methyl)-3-(4-(2-methylpyridin-4-yl)phenyl)urea (0.050 g, 0.12 mmol), zinc cyanide (0.028 g, 0.24 mmol), and tetrakis(triphenylphosphine)palladium (0.0055 g, 0.0048 mmol) in DMF (0.6 mL) was subjected to a microwave irradiation at 150° C. for 15 minutes. Then, tetrakis(triphenylphosphine)palladium (0.0055 g, 0.0048 mmol) was added and the mixture was subjected to a microwave irradiation at 170° C. for 15 minutes. Then, the mixture was filtered and the filtrate was then purified by preparative HPLC (0%-100% MeCN 0.1% TFA/H₂O 0.1% TFA) to give the desired product in a solution of MeCN 0.1% TFA and H₂O 0.1% TFA. The solvent was removed and the aqueous solution was neutralized using NaHCO₃(s). The aqueous solution was extracted with EtOAc (30 mL). The organic layer was collected, dried over MgSO₄, concentrated, and dried in vacuo to give 1-((1-(2-cyanopyridin-4-yl)cyclopentyl)methyl)-3-(4-(2-methylpyridin-4-yl)phenyl)urea as a white solid. MS (ESI, positive ion) m/z: 412 (M+1).

### Example 177

### Synthesis of 1-(1-(4-cyanophenyl)cyclobutyl)-3-(4-(2-methylpyridin-4-yl)phenyl)urea

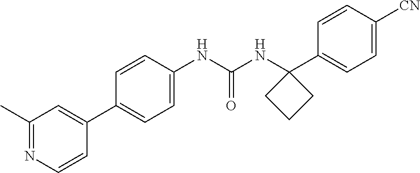

A solution of 1-(1-(4-bromophenyl)cyclobutyl)-3-(4-(2-methylpyridin-4-yl)phenyl)urea (0.100 g, 0.229 mmol), zinc cyanide (0.0218 ml, 0.344 mmol), and tetrakis(triphenylphosphine)palladium (0.0132 g, 0.0115 mmol) in DMF (1.5 mL) was subjected to a microwave irradiation at 170° C. for 15 minutes. The mixture was filtered and the filtrate was then purified by preparative HPLC (0%-100% MeCN 0.1% TFA/H₂O 0.1% TFA) to give the desired product in a solution of MeCN 0.1% TFA and H₂O 0.1% TFA. The solvent was removed and the aqueous solution was neutralized using NaHCO₃(s). The aqueous solution was extracted with EtOAc (30 mL). The organic layer was collected, dried over MgSO₄, concentrated, and dried in vacuo to give 1-(1-(4-cyanophenyl)cyclobutyl)-3-(4-(2-methylpyridin-4-yl)phenyl)urea as a white solid. MS (ESI, positive ion) m/z: 383 (M+1).

### Example 178

### Synthesis of 1-(1-(6-chloropyridin-2-yl)cyclopentyl)-3-(4-(2-methylpyridin-4-yl)phenyl)urea

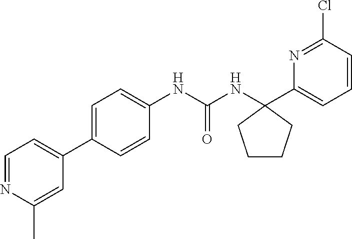

### Step 1

A solution of 1-(6-chloropyridin-2-yl)cyclopentanecarbonitrile (0.550 g, 2.66 mmol) in HCl (3 mL) was subjected to a microwave irradiation at 130° C. for 45 minutes. Then, EtOAc (20 mL) was added and the mixture was adjusted to pH ˜4 at 0° C. using saturated NaHCO₃. The mixture was then extracted with EtOAc (2×10 mL). The combined organic extracts were dried over MgSO₄ and concentrated. The residue was mixed with silica gel and the solid mixture was purified by silica gel column chromatography using ISCO instrument (solid loading, 20%-100% EtOAc/hexane) to give 1-(6-chloropyridin-2-yl)cyclopentanecarboxylic acid as a white solid. MS (ESI, positive ion) m/z: 226 (M+1).

### Step 2

A solution of 1-(6-chloropyridin-2-yl)cyclopentanecarboxylic acid (0.173 g, 0.767 mmol) and triethylamine (0.117 ml, 0.843 mmol) in THF (3 mL) at 0° C. was added ethyl chloroformate (0.0806 ml, 0.843 mmol). After addition, the mixture was stirred at room temperature for 1 h. Then, a solution of sodium azide (0.0997 g, 1.53 mmol) in H₂O (0.4 mL) was added and the mixture was stirred at room temperature for overnight. Then, H₂O (5 mL) was added and the mixture was extracted with EtOAc (2×20 mL). The combined organic extracts were dried over MgSO₄, concentrated, and dried in vacuo to give 1-(6-chloropyridin-2-yl)cyclopentanecarbonyl azide as an off-white solid, which was used without purification.

### Step 3

A solution of azido(1-(6-chloropyridin-2-yl)cyclopentyl)methanone (0.17 g, 0.66 mmol) in THF (1 mL) was subjected to a microwave irradiation at 130° C. for 15 minutes. Then, 4-(2-methylpyridin-4-yl)benzenamine (0.090 g, 0.49 mmol) was added and the mixture was subjected to a microwave irradiation at 130° C. for 15 minutes. Then, the mixture was filtered and the filtrate was purified by preparative HPLC (0%-100% MeCN 0.1% TFA/H₂O 0.1% TFA) to give a desired compound in a solution of MeCN 0.1% TFA/H₂O 0.1% TFA. The solvent was removed and the aqueous solution was neutralized using NaHCO₃(s). The aqueous solution was extracted with EtOAc (2×30 mL). The combined organic extracts were dried over MgSO₄, concentrated, and dried in vacuo to give 1-(1-(6-chloropyridin-2-yl)cyclopentyl)-3-(4-(2-methylpyridin-4-yl)phenyl)urea as a yellow solid. MS (ESI, positive ion) m/z: 407 (M+1).

### Example 179

### Synthesis of 1-(4-(2-methylpyridin-4-yl)phenyl)-3-(1-(pyridin-2-yl)cyclopentyl)urea

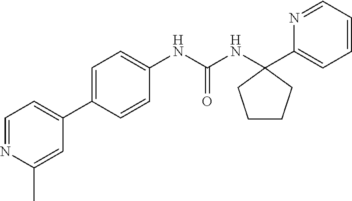

A solution of 1-(1-(6-chloropyridin-2-yl)cyclopentyl)-3-(4-(2-methylpyridin-4-yl)phenyl)urea (0.082 g, 0.20 mmol) in MeOH (1.5 mL) was added palladium hydroxide, 20 wt % pd (dry basis) on carbon, wet (0.0028 g, 0.020 mmol). The resulting mixture was then stirred at room temperature under H₂ (40 psi) for overnight. The mixture was filtered through a celite-pad and washed with MeOH (2×2 mL) and EtOAc (2×2 mL). The combined filtrates were concentrated and the residue was dissolved in DMSO (1 mL). The solution mixture was purified by preparative HPLC (0%-100% MeCN 0.1% TFA/H₂O 0.1% TFA) to give a desired compound in a solution of MeCN 0.1% TFA/H₂O 0.1% TFA. The solvent was removed and the aqueous solution was neutralized using NaHCO₃(s). The aqueous solution was extracted with EtOAc (2×10 mL). The combined organic extracts were dried over MgSO₄, concentrated, and dried in vacuo to give 1-(4-(2-methylpyridin-4-yl)phenyl)-3-(1-(pyridin-2-yl)cyclopentyl)urea as a yellow solid. MS (ESI, positive ion) m/z: 373 (M+1).

### Example 180

### Synthesis of 1-(1-(2-chloropyridin-4-yl)cyclopentyl)-3-(4-(2-methylpyridin-4-yl)phenyl)urea

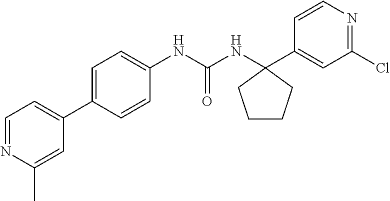

### Step 1

A solution of 1-(2-chloropyridin-4-yl)cyclopentanecarbonitrile (1.300 g, 6.290 mmol) in HCl (4 mL) was subjected to a microwave irradiation at 140° C. for 45 minutes. Then, H₂O (30 mL) was added and the solution mixture was adjusted to pH ˜5 at 0° C. using NaHCO₃(s). The mixture was then extracted with EtOAc. The combined organic extracts were dried over MgSO₄ and concentrated. The residue was then mixed with silica gel and the solid mixture was purified by silica gel column chromatography using ISCO instrument (solid loading, 20%-100% EtOAc/hexane) to give 1-(2-chloropyridin-4-yl)cyclopentanecarboxylic acid as a white solid. MS (ESI, positive ion) m/z: 226 (M+1).

### Step 2

A solution of 1-(2-chloropyridin-4-yl)cyclopentanecarboxylic acid (0.115 g, 0.510 mmol) and triethylamine (0.0780 mL, 0.561 mmol) in THF (3 mL) at 0° C. was added ethyl chloroformate (0.0536 mL, 0.561 mmol). After addition, the mixture was stirred at room temperature for 2 h. Then, a solution of sodium azide (0.0897 mL, 2.55 mmol) in H₂O (0.4 mL) was added and the mixture was stirred at room temperature for overnight. Then, H₂O (3 mL) was added and the mixture was extracted with EtOAc (2×5 mL). The combined organic extracts were dried over MgSO₄, concentrated, and dried in vacuo to give 1-(2-chloropyridin-4-yl)cyclopentanecarbonyl azide as a yellow oil, which was used without purification.

### Step 3

A solution of azido(1-(2-chloropyridin-4-yl)cyclopentyl)methanone (0.111 g, 0.443 mmol) in THF (1 mL) was subjected to a microwave irradiation at 120° C. 15 minutes. Then, 4-(2-methylpyridin-4-yl)benzenamine (0.0816 g, 0.443 mmol) was added and the mixture was subjected to a microwave irradiation at 120° C. for 15 minutes. Then, the mixture was filtered and the filtrate was purified by preparative HPLC (0%-100% MeCN 0.1% TFA/H₂O 0.1% TFA) to give a desired product in a solution of MeCN 0.1% TFA/H₂O 0.1% TFA. The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-(1-(2-chloropyridin-4-yl)cyclopentyl)-3-(4-(2-methylpyridin-4-yl)phenyl)urea as a light yellow solid. MS (ESI, positive ion) m/z: 407 (M+1).

### Example 181

### Synthesis of 1-(4-(2-methylpyridin-4-yl)phenyl)-3-(1-(pyridin-4-yl)cyclopentyl)urea

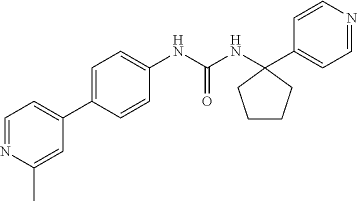

A solution of 1-(1-(2-chloropyridin-4-yl)cyclopentyl)-3-(4-(2-methylpyridin-4-yl)phenyl)urea (0.071 g, 0.17 mmol) in MeOH (1 mL) and EtOAc (0.3 mL) was added palladium hydroxide, 20 wt % pd (dry basis) on carbon, wet (0.0074 g, 0.052 mmol). The resulting mixture was then stirred at room temperature under H₂ (40 psi) for overnight. Then, the mixture was filtered through a celite and the celite was washed with MeOH (2×10 mL) and EtOAc (1×10 mL). The combined filtrates were concentrated and the residue was dissolved in DMSO (0.8 mL). The solution mixture was purified by preparative HPLC (0%-100% MeCN 0.1% TFA/H₂O 0.1% TFA) to give a desired product in a solution of MeCN 0.1% TFA/H₂O 0.1% TFA. The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-(4-(2-methylpyridin-4-yl)phenyl)-3-(1-(pyridin-4-yl)cyclopentyl)urea as a light yellow solid. MS (ESI, positive ion) m/z: 373 (M+1).

### Example 182

### Synthesis of 1-(1-(5-bromopyridin-3-yl)cyclopentyl)-3-(4-(2-methylpyridin-4-yl)phenyl)urea

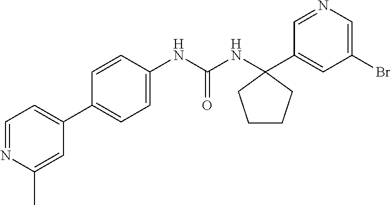

### Step 1

A solution of 3,5-dibromopyridine (14.9 g, 63.1 mmol) and cyclopentanecarbonitrile (3.29 ml, 31.5 mmol) in THF (100 mL) at 0° C. was added sodium bistrimethylsilyl amide 1M in THF (34.7 ml, 34.7 mmol). After addition, the mixture was stirred at room temperature for 48 h under N₂. Then, H₂O (100 mL) was added slowly and the mixture was extracted with EtOAc (200 mL). The organic layer was collected, dried over MgSO₄, and concentrated in vacuo to give 1-(5-bromopyridin-3-yl)cyclopentanecarbonitrile as a brown solid which was used without purification. MS (ESI, positive ion) m/z: 251 (M+1).

### Step 2

A solution of 1-(5-bromopyridin-3-yl)cyclopentanecarbonitrile (7.92 g, 31.5 mmol) in HCl (40 mL) and H₂O (5 mL) was subjected to a microwave irradiation at 170° C. for 15 minutes. Then, H₂O (150 mL) was added slowly and the mixture was adjusted to pH ˜10 at 0° C. using 10 N NaOH solution. The mixture was then extracted with EtOAc and the aqueous layer was collected. Then, the aqueous layer was adjusted to pH ˜4 at 0° C. using concentrated HCl. The aqueous layer was then extracted with EtOAc (2×100 mL). The combined organic extracts were dried over MgSO₄ and concentrated. The residue was mixed with silica gel and the solid mixture was purified by silica gel column chromatography using ISCO instrument (solid loading, 10%-100% EtOAc/hexane) to give 1-(5-bromopyridin-3-yl)cyclopentanecarboxylic acid as an off-white solid. MS (ESI, positive ion) m/z: 270 (M+1).

### Step 3

A solution of 1-(5-bromopyridin-3-yl)cyclopentanecarboxylic acid (0.470 g, 1.74 mmol) and triethylamine (0.266 ml, 1.91 mmol) in THF (5 mL) at 0° C. was added ethyl chloroformate (0.183 ml, 1.91 mmol) under N₂. After addition, the mixture was stirred at room temperature for 4 h. Then, a solution of sodium azide (0.226 g, 3.48 mmol) in H₂O (0.5 mL) was added and the mixture was stirred at room temperature for overnight. Then, H₂O (10 mL) was added and the mixture was extracted with EtOAc (2×10 mL). The combined organic extracts were dried over MgSO₄, concentrated, and dried in vacuo to give 1-(5-bromopyridin-3-yl)cyclopentanecarbonyl azide as a brown solid which was used without purification.

### Step 4

A solution of azido(1-(5-bromopyridin-3-yl)cyclopentyl)methanone (0.420 g, 1.42 mmol) in THF (1.5 mL) was subjected to a microwave irradiation at 130° C. for 15 minutes. Then, 4-(2-methylpyridin-4-yl)benzenamine (0.262 g, 1.42 mmol) was added and the mixture was subjected to a microwave irradiation at 120° C. for 15 minutes. Then, the mixture was filtered and the filtrate was purified by preparative HPLC (0%-100% MeCN 0.1% TFA/H₂O 0.1% TFA) to give a desired product in a solution of MeCN 0.1% TFA/H₂O 0.1% TFA. The solvent was removed and the aqueous solution was neutralized using NaHCO₃(s). The aqueous solution was extracted with EtOAc (2×100 mL). The combined organic extracts were dried over MgSO₄, concentrated, and dried in vacuum to give 1-(1-(5-bromopyridin-3-yl)cyclopentyl)-3-(4-(2-methylpyridin-4-yl)phenyl)urea as a yellow solid. MS (ESI, positive ion) m/z: 451 (M+1).

Proceeding as described in Example 182 above, the following compounds were prepared.

| | | | MS: ESI |
|---|---|---|---|
| | | | m/z |
| Examples | Structure | Name | (M + 1) |
| 183 | 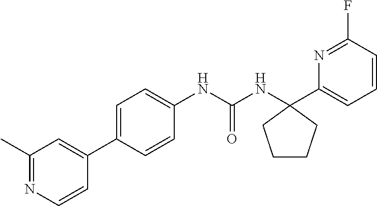 | 1-(1-(6-fluoropyridin-2- yl)cyclopentyl)-3-(4-(2- methylpyridin-4-yl)phenyl)urea | 391 |
| 184 | 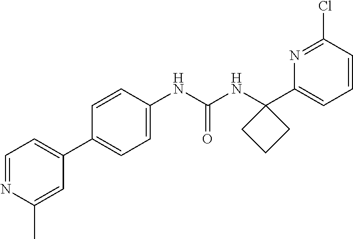 | 1-(1-(6-chloropyridin-2- yl)cyclobutyl)-3-(4-(2- methylpyridin-4-yl)phenyl)urea | 393 |

### Example 185

### Synthesis of 1-(4-(2-methylpyridin-4-yl)phenyl)-3-(1-(pyridin-3-yl)cyclopentyl)urea

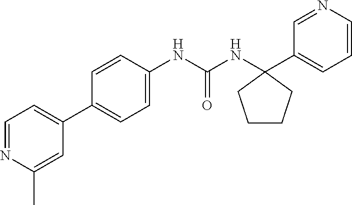

A solution of 1-(1-(5-bromopyridin-3-yl)cyclopentyl)-3-(4-(2-methylpyridin-4-yl)phenyl)urea (0.120 g, 0.266 mmol) in MeOH (1 mL) was added palladium hydroxide, 20 wt % pd (dry basis) on carbon, wet (0.00373 g, 0.0266 mmol). Then, the mixture was stirred at room temperature under H₂ (40 psi) for 2 h. Then, the mixture was filtered through a celite and the celite was washed with MeOH (2×10 mL). The combined filtrates were concentrated and the residue was dissolved in a solution of MeOH (0.5 mL) and DMSO (0.5 mL). The solution mixture was then purified by preparative HPLC (0%-100% MeCN 0.1% TFA/H₂O 0.1% TFA) to give a desired product in a solution of MeCN 0.1% TFA/H₂O 0.1% TFA. The solvent was removed and the aqueous solution was neutralized using NaHCO₃(s). The aqueous solution was extracted with EtOAc (2×20 mL). The combined organic extracts were dried over MgSO₄, concentrated, and dried in vacuo to give 1-(4-(2-methylpyridin-4-yl)phenyl)-3-(1-(pyridin-3-yl)cyclopentyl)urea as a light yellow solid. MS (ESI, positive ion) m/z: 373 (M+1).

### Example 186

### Synthesis of 1-(4-(2-methylpyridin-4-yl)phenyl)-3-(2-phenylbutan-2-yl)urea

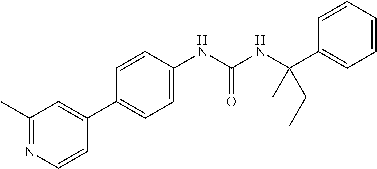

### Step 1

A solution of methyl iodide (2.0 ml, 34 mmol) and 2-phenylbutyronitrile (5.0 ml, 34 mmol) in toluene (75 mL) at 0° C. was added sodium bis(trimethylsilyl)-amide, 1.0 M in THF (34 ml, 34 mmol). After addition, the mixture was stirred at 0° C. for 2 h and at room temperature for overnight. Then, H₂O (50 mL) was added and the mixture was stirred at room temperature for 5 minutes. The organic layer was collected and the aqueous layer was extracted with EtOAc (2×50 mL). The combined organic extracts were dried over MgSO₄ and concentrated. The residue was mixed with silica gel and the solid mixture was purified by silica gel column chromatography using ISCO instrument (solid loading, 0%-100% EtOAc/hexane) to give 1-(4-(2-methylpyridin-4-yl)phenyl)-3-(2-phenylbutan-2-yl)urea as a yellow oil.

### Step 2

A solution of 2-methyl-2-phenylbutanenitrile (3.500 g, 21.98 mmol) in HCl (20 mL) and H₂O (4 mL) was subjected to a microwave irradiation at 150° C. for 2 h. Then, EtOAc (50 mL) and H₂O (50 mL) were added and the mixture was stirred at room temperature for 15 minutes. The organic layer was collected and the aqueous layer was extracted with EtOAc (1×50 mL). The combined organic extracts were dried over MgSO₄ and concentrated in vacuo. The residue was mixed with silica gel and the solid mixture was purified by silica gel flash column chromatography using ISCO instrument (solid loading, 0%-100% EtOAc/hexane) to give 2-methyl-2-phenylbutanoic acid as a colorless solid. MS (ESI, positive ion) m/z: 179 (M+1).

### Step 3

A solution of 2-methyl-2-phenylbutanoic acid (0.515 g, 2.89 mmol) and triethylamine (0.442 ml, 3.18 mmol) in THF (12 mL) at 0° C. was added ethyl chloroformate (0.304 ml, 3.18 mmol). After addition, the mixture was stirred at room temperature for overnight. Then, a solution of sodium azide (0.376 g, 5.78 mmol) in H₂O (0.5 mL) was added and the mixture was at room temperature for overnight. Then, H₂O (10 mL) was added and the mixture was extracted with EtOAc (2×20 mL). The combined organic extracts were dried over MgSO₄, concentrated, and dried in vacuo to give 2-methyl-2-phenylbutanoyl azide which was used without purification.

### Step 4

A solution of 1-azido-2-methyl-2-phenylbutan-1-one (0.587 g, 2.89 mmol) in THF (3 mL) was subjected to a microwave irradiation at 130° C. for 15 minutes. Then, 4-(2-methylpyridin-4-yl)benzenamine (0.532 g, 2.89 mmol) was added and the mixture was subjected to a microwave irradiation at 130° C. for 15 minutes. Then, a solution mixture was filtered and the filtrate was purified by preparative HPLC (0%-100% MeCN 0.1% TFA/H₂O 0.1% TFA) to give a desired product in a solution of MeCN/H₂O 0.1% TFA. The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-(4-(2-methylpyridin-4-yl)phenyl)-3-(2-phenylbutan-2-yl)urea as a yellow solid. MS (ESI, positive ion) m/z: 360 (M+1).

### Example 187

### Synthesis of 1-(1-(6-methoxypyridin-2-yl)cyclopentyl)-3-(4-(2-methylpyridin-4-yl)phenyl)urea

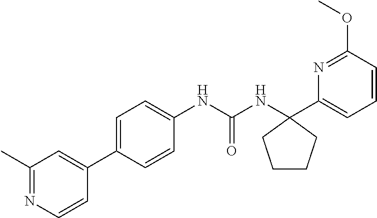

A solution of 1-(1-(6-fluoropyridin-2-yl)cyclopentyl)-3-(4-(2-methylpyridin-4-yl)phenyl)urea (0.035 g, 0.09 mmol) and sodium methoxide (0.5 M solution in methanol, 0.4 mL, 0.2 mmol) in MeOH (0.5 mL) was heated to 70° C. for 30 min and at 100° C. for 1 h. Then, H₂O (0.5 mL) was added and the mixture was filtered. The filtrate was purified by preparative HPLC (0%-100% MeCN 0.1% TFA/H₂O 0.1% TFA) to give a desired product in a solution of MeCN 0.1% TFA/H₂O 0.1% TFA). The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-(1-(6-methoxypyridin-2-yl)cyclopentyl)-3-(4-(2-methylpyridin-4-yl)phenyl)urea as a light yellow solid. MS (ESI, positive ion) m/z: 403 (M+1).

### Example 188

### Synthesis of 1-(4-hydroxy-1-phenylcyclohexyl)-3-(4-(2-methylpyridin-4-yl)phenyl)urea

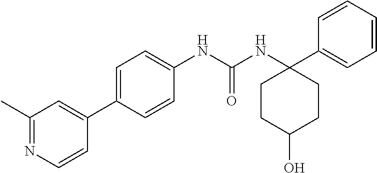

### Step 1

A solution of 1,4-cyclohexanedione monoethylene ketal (5.73 g, 36.7 mmol) in THF (100 mL) at 0° C. was added phenylmagnesium bromide 1.0 m sol in THF (40.4 mL, 40.4 mmol). After addition, the mixture was stirred at 0° C. for 4 h and at room temperature for 48 h. Then, the mixture was quenched with 100 mM sodium phosphate (pH ˜6.9, 150 mL). The mixture was then stirred at room temperature for 15 minutes. Then, the organic layer was collected and the aqueous layer was extracted with EtOAc (1×100 mL). The combined organic extracts were dried over MgSO₄ and concentrated in vacuo. The residue was mixed with silica gel and the solid mixture was purified by silica gel flash column chromatography using ISCO instrument (solid loading, 0%-100% EtOAc/hexane) to give 8-phenyl-1,4-dioxaspiro[4.5]decan-8-ol as a colorless solid.

### Step 2

A solution of 8-phenyl-1,4-dioxaspiro[4.5]decan-8-ol (2.00 g, 8.54 mmol) and sodium azide (0.752 ml, 21.3 mmol) in DCM (42 mL) at 0° C. was added trifluoroacetic acid (5.59 ml, 72.6 mmol). After addition, the mixture was stirred at 0° C. for 2 h. Then, the mixture was quenched with concentrated NH₄OH (25 mL). Then, the mixture was stirred at room temperature for 15 minutes. Then, H₂O (100 mL) was added and the mixture was extracted with EtOAc (2×100 mL). The combined organic extracts were dried over MgSO₄ and concentrated in vacuo. The residue was mixed with silica gel and the solid mixture was purified by silica gel column chromatography using ISCO instrument (solid loading, 0%-100% EtOAc/hexane) to give 8-azido-8-phenyl-1,4-dioxaspiro[4.5]decane as a yellow oil.

### Step 3

A solution of 8-azido-8-phenyl-1,4-dioxaspiro[4.5]decane (0.770 g, 2.97 mmol) and palladium, 10 wt. % on activated carbon (0.316 g, 0.297 mmol) in EtOH (20 mL) was stirred at room temperature for overnight under H₂. Then, the mixture was filtered through a celite and the celite was washed with MeOH (2×20 mL). The combined filtrate was then concentrated and dried in vacuo to give 8-phenyl-1,4-dioxaspiro[4.5]decan-8-amine as a light brown oil, which was without purification.

### Step 4

A solution of azido(4-(2-methylpyridin-4-yl)phenyl)methanone (0.670 g, 2.81 mmol) in THF (3 mL) was subjected to a microwave irradiation at 120° C. for 15 minutes. Then, 8-phenyl-1,4-dioxaspiro[4.5]decan-8-amine (0.656 g, 2.81 mmol) was added and the mixture was stirred at room temperature for overnight. The mixture was filtered and the filtrate was purified by silica gel column chromatography using ISCO instrument (solid loading, 0%-100% EtOAc/hexane) to give 1-(4-(2-methylpyridin-4-yl)phenyl)-3-(8-phenyl-1,4-dioxaspiro[4.5]decan-8-yl)urea as a white solid. MS (ESI, positive ion) m/z: 444 (M+1).

### Step 5

A solution of 1-(4-(2-methylpyridin-4-yl)phenyl)-3-(8-phenyl-1,4-dioxaspiro-[4.5]decan-8-yl)urea (0.795 g, 1.79 mmol) in acetone (7 mL) was added H₂O (3 mL) and p-toluenesulfonic acid (10.4 mL, 8.96 mmol). After addition, the mixture was stirred at room temperature for 4 h. Then, H₂O (10 mL) and EtOAc (30 mL) were added and the mixture was neutralized by Na₂CO₃(s). The neutralized aqueous was extracted with EtOAc (2×20 mL). The combined organic extracts were dried over MgSO₄ and concentrated. The residue was dissolved in DCM (1.5 mL) and the solution mixture was purified by silica gel column chromatography using ISCO instrument (0%-20% MeOH/DCM) to give 1-(4-(2-methylpyridin-4-yl)phenyl)-3-(4-oxo-1-phenylcyclohexyl)urea as a white solid. MS (ESI, positive ion) m/z: 400 (M+1).

### Step 6

A solution of 1-(4-(2-methylpyridin-4-yl)phenyl)-3-(4-oxo-1-phenylcyclohexyl)urea (0.086 g, 0.22 mmol) in THF (2 mL) was added sodium borohydride (0.0098 g, 0.26 mmol). After addition, the mixture was stirred at room temperature for 1 h. Then, the mixture was quenched with saturated NaHCO₃. Then, the mixture was extracted with EtOAc (2×20 mL). The combined organic extracts were dried over MgSO₄ and concentrated. The residue was dissolved in DMSO (1 mL) and the solution mixture was purified by preparative HPLC (0%-100% MeCN 0.1% TFA/H₂O 0.1% TFA) to give a desired product in a solution of MeCN/H₂O 0.1% TFA. The solvent was removed and the aqueous solution was neutralized using NaHCO₃(s). The aqueous solution was extracted with EtOAc. The combined extracts were dried over MgSO₄, washed with brine, concentrated, and dried in vacuo to give 1-(4-hydroxy-1-phenylcyclohexyl)-3-(4-(2-methylpyridin-4-yl)phenyl)urea as a white solid. MS (ESI, positive ion) m/z: 402 (M+1).

### Example 189

### Synthesis of 1-(4,4-difluoro-1-phenylcyclohexyl)-3-(4-(2-methylpyridin-4-yl)phenyl)urea

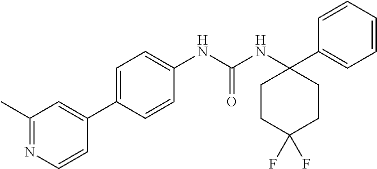

A solution of 1-(4-(2-methylpyridin-4-yl)phenyl)-3-(4-oxo-1-phenylcyclohexyl)urea (0.100 g, 0.250 mmol) at 0° C. was added (diethylamino)sulfur trifluoride (0.165 mL, 1.25 mmol). After addition, the mixture was stirred at 0° C. for 4.5 h. Then, the mixture was poured into an ice water (100 mL) and EtOAc (20 mL) was added. The mixture was stirred at room temperature for 5 minutes. The organic layer was collected and the aqueous layer was extracted with EtOAc (20 mL). The combined organic extracts were dried over MgSO₄ and concentrated. The residue was dissolved in DMSO (2 mL). The solution mixture was then purified by preparative HPLC (0%-100% MeCN 0.1% TFA/H₂O 0.1% TFA) to give a desired product in a solution of MeCN/H₂O 0.1% TFA. The solvent was removed and the aqueous solution was neutralized using NaHCO₃(s). The aqueous solution was extracted with EtOAc (20 mL). The organic layer was collected, dried over MgSO₄, concentrated, and dried in vacuo to give 1-(4,4-difluoro-1-phenylcyclohexyl)-3-(4-(2-methylpyridin-4-yl)phenyl)urea as an off-white solid. MS (ESI, positive ion) m/z: 422 (M+1).

### Example 190

### Synthesis of 14243-fluorophenyl)propan-2-yl)-3-(4-(2-methylpyridin-4-yl)phenyl)urea

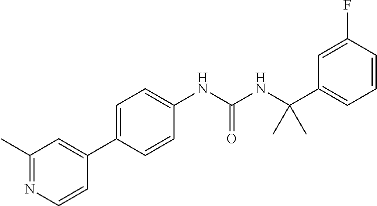

### Step 1

A solution of 2-(3-fluorophenyl)acetonitrile (5.0 g, 37.0 mmol) and methyl iodide (5.06 mL, 81.4 mmol) in THF (100 mL) at 0° C. was added sodium bis(trimethylsilyl)amide, 1.0M solution in THF (81.4 mL, 81.4 mmol). After addition, the mixture was stirred at 0° C. for 45 minutes. Then, H₂O (100 mL) and EtOAc (100 mL) were added and the mixture was stirred at room temperature for 15 minutes. The organic layer was collected and the aqueous was extracted with EtOAc (1×100 mL). The combined organic extracts were dried over MgSO₄, concentrated, and dried in vacuo to give 2-(3-fluorophenyl)-2-methylpropanenitrile as orange oil which was used without purification.

### Step 2

A solution of 2-(3-fluorophenyl)-2-methylpropanenitrile (2.00 g, 12.3 mmol) in HCl (concentrated, 10 mL) and H₂O (1 mL) was subjected to a microwave irradiation at 150° C. for 1 h. Then, H₂O (100 mL) and EtOAc (150 mL) were added and the mixture was stirred at room temperature for 10 minutes. Then, the organic layer was collected, dried over MgSO₄, and concentrated. The residue was mixed with silica gel and the solid mixture was purified by silica gel column chromatography using ISCO instrument (solid loading, 0%-100% EtOAc/hexane) to give 2-(3-fluorophenyl)-2-methylpropanoic acid as orange oil. MS (ESI, positive ion) m/z: 183 (M+1).

### Step 3

A solution of 2-(3-fluorophenyl)-2-methylpropanoic acid (0.450 g, 2.47 mmol) and triethylamine (0.344 ml, 2.47 mmol) in THF (16 mL) at 0° C. was added ethyl chloroformate (0.236 ml, 2.47 mmol). After addition, the mixture was stirred at room temperature for 3 h. Then, a solution of sodium azide (0.321 g, 4.94 mmol) in H₂O (0.7 mL) was added and the mixture was stirred at room temperature for overnight. Then, H₂O (5 mL) and EtOAc (20 mL) were added and the mixture was stirred at room temperature for 10 minutes. The organic layer was collected, dried over MgSO₄, concentrated, and dried in vacuo to give 2-(3-fluorophenyl)-2-methylpropanoyl azide as yellow oil which was used without purification.

### Step 4

A solution of 2-(3-fluorophenyl)-2-methylpropanoyl azide (0.487 g, 2.35 mmol) in THF (3 mL) was subjected to a microwave irradiation at 120° C. for 15 minutes. Then, 4-(2-methylpyridin-4-yl)benzenamine (0.433 g, 2.35 mmol) was added and the mixture was subjected to a microwave irradiation at 120° C. for 15 minutes. Then, the mixture was filtered and the filtrate was purified by preparative HPLC (0%-100% MeCN 0.1% TFA/H₂O 0.1% TFA) to give a desired product in a solution of MeCN/H₂O 0.1% TFA. The solvent was removed and the aqueous solution was neutralized using NaHCO₃(s). The aqueous solution was extracted with EtOAc. The combined extracts were dried over MgSO₄, washed with brine, concentrated, and dried in vacuo to give 1-(2-(3-fluorophenyl)propan-2-yl)-3-(4-(2-methylpyridin-4-yl)phenyl)urea as yellow solid. MS (ESI, positive ion) m/z: 364 (M+1).

### Example 191

### Synthesis of 1-(4-(2-methylpyridin-4-yl)phenyl)-3-(2-(thiophen-3-yl)propan-2-yl)urea

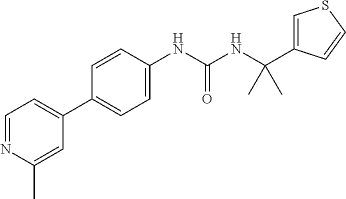

### Step 1

A flask containing anhydrous cerium (III) chloride (10.2 g, 41.2 mmol) was flushed with nitrogen several times and placed under high vacuum. The solid was heated with a heat gun for several minutes to remove any excess water. The flask was re-filled with nitrogen and allowed to cool. THF (80 mL) was added and the suspension was stirred at room temperature for 18 h. The suspension was cooled to −78° C. and methyllithium, 1.0 M solution (41.2 mL, 41.2 mmol) was added. After stirring for 1 h, thiophene-3-carbonitrile (1.50 g, 13.7 mmol) was added. The mixture was stirred for 5 h at −78° C., then allowed to slowly warm to room temperature over 16 h. The mixture was cooled back to −78° C., then treated with conc. NH₄OH (30 mL). After warming to room temperature, the mixture was diluted with water and extracted with CH₂Cl₂. The combined organics were dried over Na₂SO₄, filtered and concentrated. The residue was then dissolved in CH₂Cl₂ and extracted with 2N HCl. The combined aqueous layers were basified with 5 N NaOH, and extracted with CH₂Cl₂. The combined organics were dried over Na₂SO₄, filtered and concentrated to give brown oil. MS (ESI, pos. ion) m/z: 125 (M−16).

### Step 2

A mixture of 1,1′-carbonyldiimidazole (194 mg, 1.2 mmol) and 4-(2-methylpyridin-4-yl)benzenamine (200 mg, 1.1 mmol) in CH₂Cl₂ (10 mL) was stirred at room temperature for 5 min. The solution was treated with 2-(thiophen-3-yl)propan-2-amine (153 mg, 1.1 mmol) and triethylamine (0.45 mL, 3.2 mmol) and stirred for 6 h at room temperature. The mixture was diluted with saturated aqueous NaHCO₃ and extracted with CH₂Cl₂. The combined organics were dried over Na₂SO₄, filtered and concentrated. The residue was recrystallized from MeOH to yield 1-(4-(2-methylpyridin-4-yl)phenyl)-3-(2-(thiophen-3-yl)propan-2-yl)urea. MS (ESI pos. ion) m/z: 352 (M+1).

### Example 192

### Synthesis of 1-(2-(benzofuran-5-yl)propan-2-yl)-3-(4-(2-methylpyridin-4-yl)phenyl)urea

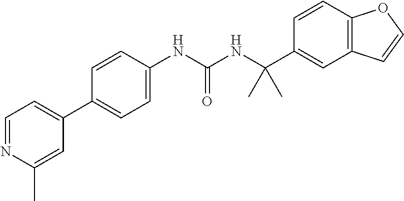

### Step 1

A flask containing anhydrous cerium (III) chloride (5.17 g, 21.0 mmol) was flushed with nitrogen several times and placed under high vacuum. The solid was heated with a heat gun for several minutes to remove any excess water. The flask was re-filled with nitrogen and allowed to cool. THF (80 mL) was added and the suspension was stirred at room temperature for 3 h. The suspension was cooled to −78° C. and methyllithium, 3.0 M solution in Et₂O (6.99 mL, 21.0 mmol) was added. After stirring for 1 h, benzofuran-5-carbonitrile (1.00 g, 6.99 mmol) was added. The mixture was stirred for 5 h at −78° C., then allowed to slowly warm to room temperature over 16 h. The mixture was cooled back to −78° C., then treated with conc. NH₄OH (30 mL). After warming to room temperature, the mixture was diluted with H₂O and extracted with CH₂Cl₂. The combined organics were dried over Na₂SO₄, filtered and concentrated. The residue was then dissolved in CH₂Cl₂ and extracted with 2 N HCl. The combined aqueous layers were basified with 5 N NaOH, and extracted with CH₂Cl₂. The combined organics were dried over Na₂SO₄, filtered and concentrated to yield 2-(benzofuran-5-yl)propan-2-amine MS (ESI, pos. ion) m/z: 159 (M−16).

### Step 2

A mixture of 4-(2-methylpyridin-4-yl)benzenamine (200 mg, 1.1 mmol) and 1,1′-carbonyldiimidazole (194 mg, 1.2 mmol) was stirred for five minutes, then treated with 2-(benzofuran-5-yl)propan-2-amine (190 mg, 1.1 mmol) and triethylamine (454 μl, 3.2 mmol) and stirred for 5 h. After cooling to room temperature, the mixture was diluted with sat aq. NaHCO₃ and extracted with 25% i-PrOH/CHCl₃ (3×). The combined organics were dried over Na₂SO₄, filtered and concentrated. The residue was recrystallized from MeOH. The solid was dissolved in DMSO and recrystallized to yield pure 1-(2-(benzofuran-5-yl)propan-2-yl)-3-(4-(2-methylpyridin-4-yl)phenyl)urea. MS (ESI pos. ion) m/z: 386 (M+1).

### Example 193

### Synthesis of 1-(2-(benzo[b]thiophen-5-yl)propan-2-yl)-3-(4-(2-methylpyridin-4-yl)phenyl)urea

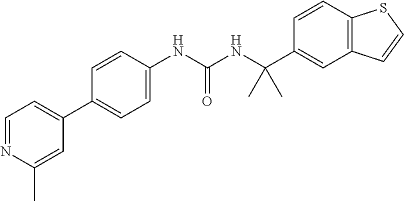

### Step 1

A flask containing anhydrous cerium (III) chloride (4.64 g, 18.8 mmol) was flushed with nitrogen several times and placed under high vacuum. The solid was heated with a heat gun for several minutes to remove any excess water. The flask was re-filled with nitrogen and allowed to cool. THF (80 mL) was added and the suspension was stirred at room temperature for 3 h. The suspension was cooled to −78° C. and methyllithium (3.0 M solution in Et₂O, 6.28 mL, 18.8 mmol) was added. After stirring for 1 h, benzo[b]thiophene-6-carbonitrile (1.00 g, 6.28 mmol) was added. The mixture was stirred for 5 h at −78° C., then allowed to slowly warm to room temperature over 16 h. The mixture was cooled back to −78° C., then treated with conc. NH₄OH (30 mL). After warming to room temperature, the mixture was diluted with H₂O and extracted with CH₂Cl₂. The combined organics were dried over Na₂SO₄, filtered and concentrated. The residue was then dissolved in CH₂Cl₂ and extracted with 2 N HCl. The combined aqueous layers were basified with 5 N NaOH, and extracted with CH₂Cl₂. The combined organics were dried over Na₂SO₄, filtered and concentrated to yield 2-(benzo[b]thiophen-5-yl)propan-2-amine. MS (ESI pos. ion) m/z: 175 (M−16).

### Step 2

A mixture of 4-(2-methylpyridin-4-yl)benzenamine (200 mg, 1.1 mmol) and 1,1′-carbonyldiimidazole (194 mg, 1.2 mmol) in CH₂Cl₂ (10 mL) was stirred for 5 minutes, then treated with 2-(benzo[b]thiophen-5-yl)propan-2-amine (208 mg, 1.1 mmol) and triethylamine (0.45 mL, 3.2 mmol) and stirred for 5 h. After cooling to room temperature, the mixture was diluted with saturated aqueous NaHCO₃ and extracted with 25% i-PrOH/CHCl₃. The combined organics were dried over Na₂SO₄, filtered and concentrated. The residue was recrystallized from MeOH to yield pure 1-(2-(benzo[b]thiophen-5-yl)propan-2-yl)-3-(4-(2-methylpyridin-4-yl)phenyl)urea. MS (ESI pos. ion) m/z: 402 (M+1).

### Example 194

### Synthesis of 1-(2-(5-bromothiophen-2-yl)propan-2-yl)-3-(4-(2-methylpyridin-4-yl)phenyl)urea

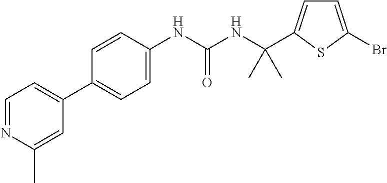

### Step 1

A solution of diisopropylamine (19.9 mL, 141.3 mmol) in THF (90 mL) in an oven-dried round-bottomed flask was cooled to −5° C. under nitrogen, and was treated with butyllithium (2.5M solution in hexanes, 56.5 mL, 141.3 mmol) in a dropwise manner. The reaction was stirred at −5° C. under nitrogen. After 30 minutes, a solution of thiophene-2-acetic acid (5.02 g, 35.3 mmol) in THF (10 mL) was added to the reaction in a dropwise fashion. After another 30 minutes, iodomethane (13.7 mL, 22.0 mmol) was added in a dropwise fashion. The reaction was slowly allowed to warm to 23° C. Ater 16 h, the suspension was diluted with EtOAc (150 mL) and washed with 10% aqueous hydrochloric acid solution (100 mL) and brine (100 mL), dried over MgSO₄, concentrated in-vacuo and purified by silica gel chromatography using methanol/dichloromethane as the eluant affording 2-methyl-2-(thiophen-2-yl)propanoic acid as an off-white solid. MS (ESI pos. ion) m/z: 171 (M+1).

### Step 2

A solution of 2-methyl-2-(thiophen-2-yl)propanoic acid (264 mg, 1.551 mmol) in dichloromethane (10 mL) was treated with n-bromosuccinimide (345 mg, 1.939 mmol). The reaction was stirred at 23° C. under nitrogen. After 21 h, the reaction was diluted with EtOAc (75 mL) and washed with water (50 mL) and brine (50 mL), dried over MgSO₄, concentrated in vacuo and purified by silica gel chromatography using methanol/dichloromethane as the eluant affording 2-(5-bromothiophen-2-yl)-2-methylpropanoic acid as an off-white solid. MS (ESI pos. ion) m/z: 249 (M+1).

### Step 3

A solution of 2-(5-bromothiophen-2-yl)-2-methylpropanoic acid (266 mg, 1.068 mmol) and triethylamine (0.446 mL, 3.203 mmol) in THF (10 mL) was cooled to 0° C. and was treated with ethyl chloroformate (0.204 mL, 2.135 mmol). After 1 h, a solution of sodium azide (208 mg, 3.203 mmol) in water (0.9 mL) was added, and the reaction was stirred at 23° C. After 17 h, the reaction was diluted with EtOAc (100 mL) and washed with water (50 mL) and brine (50 mL), dried over MgSO₄, concentrated in-vacuo, affording 2-(5-bromothiophen-2-yl)-2-methylpropanoyl azide as yellow oil which was used without purification.

### Step 4

A solution of 1-azido-2-(5-bromothiophen-2-yl)-2-methylpropan-1-one (290 mg, 1.058 mmol) in THF (2 mL) was heated to 120° C. in a microwave for 15 minutes. 4-(2-methylpyridin-4-yl)benzenamine (195 mg, 1.058 mmol) was added, followed by THF (0.5 mL), and reaction was heated in a microwave at 120° C. for 15 minutes. The reaction was concentrated in vacuo and purified by silica gel chromatography using methanol/dichloromethane as the eluant, affording 1-(2-(5-bromothiophen-2-yl)propan-2-yl)-3-(4-(2-methylpyridin-4-yl)phenyl)urea as a light yellow solid. MS (ESI pos. ion) m/z: 430 (M+1).

### Example 195

### Synthesis of 1-((1-(2-methoxyphenyl)cyclobutyl)methyl)-3-(4-(2-methylpyridin-4-yl)phenyl)urea

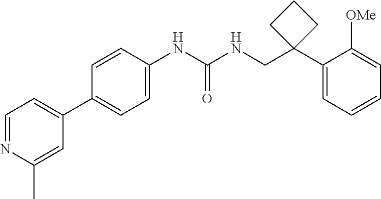

A solution of azido(4-(2-methylpyridin-4-yl)phenyl)methanone (50 mg, 0.210 mmol) and 1,4-dioxane (2 mL) was heated at 120° C. for 10 min. 1-(2-Methoxyphenyl) cyclobutyl)methanamine in 1,4-dioxane (2 mL) was added and the reaction was allowed to cool to room temperature with stirring. After 30 minutes, the reaction was concentrated in vacuo then purified by silica-gel chromatography using MeOH-DCM as the eluant to give 1-((1-(2-methoxyphenyl)cyclobutyl)methyl)-3-(4-(2-methylpyridin-4-yl)phenyl)urea as a light yellow oil. MS (ESI pos. ion) m/z: 402 (M+1).

### Biological Examples

### Example 1

### Cell-Based . . . In Vitro Assays

GSM cell-based assays were designed to measure the modulation of Aβ 42 from HEK 293 cells over-expressing APP.

Three simultaneous cell-based assays, namely Aβ 42 inhibition or reduction, Aβ 40 inhibition or reduction and a measurement of cell viability of the cells from which the Aβ 42/40 readout was obtained, together gave an interpretation of gamma secretase modulation (GSM). The assays measure Aβ 42 and Aβ 40 from conditioned medium of test compound treated HEK 293 cells.

HEK293 cells stably expressing full length Amyloid Precursor Protein (APP) were plated at a density of 100K cells/well in 96 well plates (Costar). The cells were cultivated for 6 hours at 37° C. and 5% CO₂ in DMEM supplemented with 10% FBS. The test compounds were then added to cells in 10-point dose response concentrations with the starting concentration being 10 μM. The compounds were diluted from stock solutions in DMSO and the final DMSO concentration of the test compounds on cells was 0.1%. After 24 hours of incubation with the test compounds the supernatant conditioned media was collected and the Aβ 42, Aβ 40 levels were determined using a sandwich ELISA. A cell viability test (CellTiter-Blue Cell Viability assay, Promega, using the manufacturers protocol) on the cells from which the conditioned medium was harvested for Aβ 42 or Aβ 40 readouts gave an indication of cell survivability as a possible reason for false positive Aβ 42 or 40 reduction or inhibition readout. The IC₅₀ of the compound (for either Aβ 42 or Aβ 40) was calculated from the percent of control or percent inhibition of Aβ 42 or Aβ 40 as a function of the concentration of the test compound.

The sandwich ELISA to detect Aβ 42 or Aβ 40 was performed in 96 well microtiter plates, which were pre-treated with goat anti-rabbit IgG (Pierce). The capture and detecting antibody pair that were used to detect Aβ 42 and Aβ 40 from cell supernatants were rabbit monoclonal Antibody 42 (RabMAb 42) and affinity purified polyclonal Antibody 40 (pAbeta40, Biosource) as capture antibodies and biotinylated 6E10 monoclonal (Signet Labs Inc.) as detection antibody. The optimal concentration for RabMAb 42 was 1 μg/ml in Superblock/TBS (Pierce) that was supplemented with 0.05% Tween 20 (Sigma). The optimal concentration for the pAb40 antibody was 3 μg/ml in Superblock/TBS (Pierce) that was supplemented with 0.05% Tween 20 (Sigma). Optimal concentration for the detection antibody 6E10-biotinylated was 0.5 μg/ml in Superblock/TBS (Pierce) that had been supplemented with 2% normal goat serum and 2% normal mouse serum.

Cellular supernatants were incubated with the capture antibody for 16-20 hours at 4° C., followed by 3 wash steps in TBS-tween (0.05%). The detecting antibody incubation was for 3 hours at 4° C., again followed by the wash steps as described previously. The final readout of the ELISA is Time-Resolved Fluorescence (counts per minute) using Delfia reagents Streptavidin-Europium and Enhancement solutions (Perkin Elmer) and the EnVision Multilabel plate reader (Perkin Elmer).

The approximate IC₅₀ value of a representative number of compounds of Formula (I) for the reduction of Ab42 in this assay is provided in the tables below.

| | IC₅₀ | | IC₅₀ | | IC₅₀ |
|---|---|---|---|---|---|
| Cpd # | (uM) | Cpd # | (uM) | Cpd # | (uM) |
| T-1-1 | 0.224 | T-1-74 | 3.33 | T-1-84 | 10 |
| T-1-3 | 0.131 | T-1-75 | 3.13 | T-1-85 | 10 |
| T-1-5 | 0.748 | T-1-76 | 2.68 | T-1-86 | 8.62 |
| T-1-6 | 1.517 | T-1-78 | 2.36 | T-1-87 | 0.62 |
| T-1-8 | 1.082 | T-1-81 | 10 | T-1-88 | 1.00 |
| T-1-90 | 9.93 | T-1-111 | 10 | T-1-120 | 3.63 |
| T-1-92 | 10 | T-1-113 | 10 | T-1-121 | 1.04 |
| T-1-93 | 5.45 | T-1-116 | 10 | T-1-123 | 2.37 |
| T-1-94 | 10 | T-1-117 | 2.58 | T-1-125 | 1.11 |
| T-1-95 | 10 | T-1-118 | 0.311 | T-1-126 | 1.08 |
| T-1-128 | 10 | T-1-141 | 1.30 | T-1-151 | 1.82 |
| T-1-129 | 1.33 | T-1-142 | 4.15 | T-1-152 | 10 |
| T-1-131 | 10 | T-1-144 | 1.39 | T-1-157 | 10 |
| T-1-136 | 3.33 | T-1-147 | 10 | T-1-162 | 10 |
| T-1-138 | 1.10 | T-1-149 | 1.78 | T-1-163 | 10 |
| T-1-164 | 3.33 | T-1-192 | 10 | T-1-200 | 4.35 |
| T-1-165 | 0.644 | T-1-193 | 10 | T-1-207 | 0.27 |
| T-1-168 | 3.33 | T-1-197 | 10 | T-1-215 | 10 |
| T-1-172 | 1.94 | T-1-198 | 5.91 | T-1-217 | 0.76 |
| T-1-191 | 0.955 | T-1-199 | 10 | T-1-218 | 0.56 |
| T-1-219 | 10 | T-1-226 | 1.50 | T-1-222 | 0.64 |
| T-1-221 | 10 | T-1-227 | 0.70 | T-1-223 | 10 |
| T-1-224 | 0.21 | | | | |

| Cpd # | IC₅₀ (nM) | Cpd # | IC₅₀ (nM) |
|---|---|---|---|
| T-2-1 | 0.36 | T-2-10 | 0.75 |
| T-2-3 | 0.201 | T-2-12 | 3.41 |
| T-2-5 | 1.12 | T-2-18 | 0.76 |
| T-2-6 | 1.48 | T-2-19 | 2.31 |
| T-2-8 | 2.66 | T-2-23 | 2.75 |
| T-2-24 | 10 | T-2-33 | 0.88 |
| T-2-35 | 2.5 | T-2-36 | 0.13 |

T-1-1 means Compound Table 1, cpd. 1 and T-2-1 means Compound Table 2, cpd. 1.

### Example 2

### Quantification of Aβ42 Peptides in Brain, CSF and Plasma . . . In Vivo Assay

Four to six month old Sprague Dawley rats (200-250 g) were administered compound of the Invention formulated at 10 ml/kg in 2% HPMC (hydroxypropyl methylcellulose), 1% Tween80, pH 2.2 by oral gavage. One to seventy two hours following the administration of the compound, the animals were sacrificed. Cerebrospinal fluid was isolated from the cisterna magna, blood was taken via cardiac puncture and brains were removed and divided into quarters. Samples were frozen on dry ice and stored at −80° C. Brains were homogenized in 10 volumes (w/v) of 0.2% diethylamine (DEA) in 50 mM NaCl and centrifuged at 355,000 g, 4° C. for 30 minutes. CSF or brain supernatants were then analyzed for the presence of Aβ42 peptide by specific sandwich ELISA assays based on ECL (Electrochemiluminescence) technology as described below.

Rat Aβ 42 peptide was quantified using biotinylated 4G8 antibody (Signet) for capture and ruthenylated ConFab42 antibody (ConFab42 was generated at Biosite, Inc. Briefly, phage libraries expressing Fabs (Fragment Antigen Binding portion of an antibody) were prepared from mice immunized with two peptides, Aβ34-42 and Aβ35-43. Fab libraries were then screened for their ability to bind to Aβ42 with less than 0.1% cross-reactivity to Aβ40). Initially the MSD 96-well avidin plates were coated with biotinylated 4G8 capture antibody (0.25 ug/well in PBS) by incubation for 1.5 hours at 4° C. After a wash and blocking step, 25 ul of the either brain homogenate (CSF or plasma) or synthetic Aβ peptide standard was added to the MSD plate followed by addition of 25 ul of ruthenylated ConFab42 (5 ug/ml in MSD antibody diluent) for overnight incubation at 4° C. with shaking. The standards were made using serial dilutions of synthetic human Aβ 42 peptide (Anaspec) and final concentrations ranged from 6.25-800 pg/ml in brain homogenization buffer (DEA or TBS Triton) or MSD Tris lysis buffer for rat CSF and plasma analysis. Rat brain and plasma were run neat in the assay whereas CSF was diluted 1:5 in MSD Tris lysis buffer. The 96-well plates were washed twice the next morning and read in the MSD Sector Imager 6000 to quantify the ECL signals in the wells after addition of 150 ul/well of MSD read buffer T with surfactant 1×. The data were analyzed using commercially available software such as Softmax Pro, Excel and GraphPad Prism programs.

### Formulation Examples

The following are representative pharmaceutical formulations containing a compound of Formula (I).

### Tablet Formulation

The following ingredients are mixed intimately and pressed into single scored tablets.

| | Quantity per tablet |
|---|---|
| Ingredient | mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per capsule |
|---|---|
| Ingredient | mg |
| compound of this invention | 200 |
| lactose spray dried | 148 |
| magnesium stearate | 2 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

1. A compound of Formula (I): 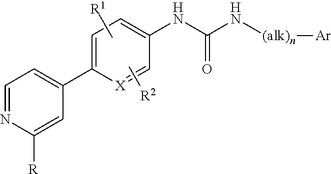
where:
X is —CH— or —N—;
n is 0 or 1;
alk is a straight or branched alkyl of 1 to 6 carbon atoms where one, two, or three hydrogen atoms of the alkyl chain are replaced by alkoxy, hydroxyl or halo;
R is alkyl;
R¹ and R² are independently hydrogen, alkyl, alkoxy, hydroxy, or halo;
Ar is:
(i) aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{C} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or
(ii) a group of formula (a): 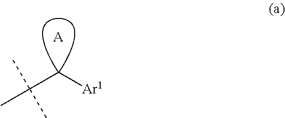
ring A is cycloalkyl optionally substituted with halo, hydroxyl, alkoxy, oxo, or haloalkyl; or monocyclic heterocyclyl wherein if the heterocyclyl ring contains a nitrogen ring atom, the nitrogen atom is optionally substituted with alkyl, or acyl, acyloxycarbonyl;
Ar¹ is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; or
a pharmaceutically acceptable salt thereof provided the compound of Formula (I) is not:

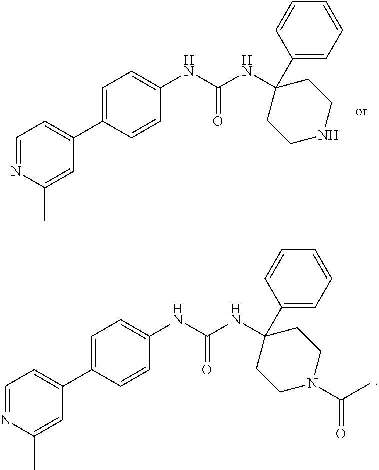

2. The compound of claim 1 wherein Ar is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino.

3. The compound of claim 1 wherein Ar is a group of formula (a): 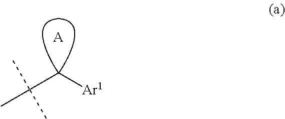
ring A is cycloalkyl optionally substituted with halo, hydroxyl, alkoxy, oxo, or haloalkyl; or monocyclic heterocyclyl wherein if the heterocyclyl ring contains a nitrogen ring atom, the nitrogen atom is optionally substituted with alkyl, or acyl, acyloxycarbonyl;
Ar¹ is aryl, heteroaryl, cycloalkyl, fused cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino and R^{b} and R^{c} are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, acylamino, aryl, heteroaryl, cycloalkyl, heterocyclyl, aralkyl, heteroaralkyl, aryloxy, heteroaryloxy, or cycloalkoxy, or when R^{b} and R^{c} are on adjacent atoms they can combine to form methylenedioxy or ethylenedioxy; where the aromatic or alicyclic ring in R^{a}, R^{b} and R^{c} is optionally substituted with R^{d}, R^{e} or R^{f} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino.

4. The compound of claim 2 where n is 0.

5. The compound of claim 2 where n is 1.

6. The compound of claim 2 where alk is —CH₂—, —(CH₂)₂—, —CHCH₃—, —C(CH₃)₂—.

7. The compound of claim 2 wherein the compound of Formula (I) has the structure: 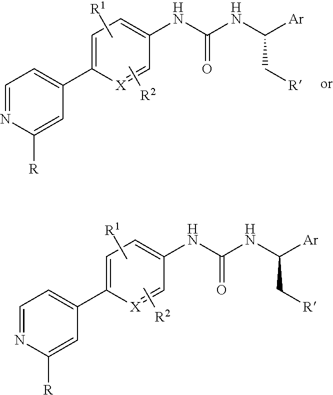
where R′ is methyl, ethyl, n-propyl, n-butyl, or isobutyl.

8. The compound of claim 1 where X is —N—.

9. The compound of claim 1 wherein X is —CH—.

10. The compound of claim 7 wherein R¹ and R² are hydrogen and X is —CH—.

11. The compound of claim 10 wherein R is methyl.

12. The compound of claim 1 where Ar is aryl optionally substituted with R^{a}, R^{b}, or R^{c}.

13. The compound of claim 1 wherein Ar is aryl, heteroaryl, cycloalkyl, or heterocyclyl where each of the aforementioned ring is optionally substituted with R^{a}, R^{b} or R^{c} where R^{a} is alkyl, halo, haloalkyl, haloalkoxy, alkylthio, cyano, alkoxy, or acyl and R^{b} and R^{c} are independently selected from alkyl, halo, haloalkyl, aryl, heteroaryl, heterocyclyl, or aryloxy; where the aromatic or alicyclic ring in R^{a}, R^{b} and R^{c} is optionally substituted with R^{d}, R^{e} or R^{f} which are independently selected from alkyl, halo, cyano, or alkoxy.

14. The compound of claim 1 wherein Ar is 3-morpholin-4-ylphenyl; 4-methylphenyl; 4-chiorophenyl; 2-chlorophenyl; 3-chlorophenyl; 3-bromophenyl; 2-bromophenyl; 2-fluorophenyl; 3-fluorophenyl; 2,4-dichlorophenyl; 4-methoxyphenyl; 3-difluoromethoxyphenyl; 5-bromo-2-fluorophenyl; phenyl; 2-methylphenyl; 3-n-butoxyphenyl; 2-fluoro-5-cyanophenyl; 5-cyano-2-methylphenyl; 5-chloro-2-fluorophenyl; 3-ethoxyphenyl; 2-methyl-5-trifluoromethylphenyl; 3-n-propoxyphenyl; 2-n-propoxyphenyl; 3-tert-butylphenyl; 2-pyridin-3-ylphenyl; 5-bromo-2-methylphenyl; 2-n-propylphenyl; 5-chloro-2-methylphenyl; 3-ethoxyphenyl; 2-fluoro-5-trifluoromethylphenyl; 2-ethylphenyl; 3-methylthiophenyl; 2,5-difluorophenyl; 5-fluoro-2-methoxyphenyl; 2-cyanophenyl; 2-benzoylphenyl; 2-trifluoromethylphenyl; 5-fluoro-2-methylphenyl; 2-chloro-5-methoxyphenyl; 2-chloro-5-methylphenyl; 2-phenoxyphenyl; 3-cyanophenyl; 2-acetylphenyl; 2-cyano-5-methylphenyl; 3-isopropoxyphenyl; 3-benzoylphenyl; 2-chloro-5-cyanophenyl; 3-phenoxyphenyl; 2-methoxy-5-methylphenyl; 2-chloro-5-trifluoromethylphenyl; 2,5-dichlorophenyl; 3-methoxyphenyl; 3-ethylphenyl; 2-methylphenyl; 2-methylthiophenyl; 2-methoxy-5-trifluoromethylphenyl; 5-chloro-2-cyanophenyl; 2-ethoxyphenyl; 2-isopropylphenyl; 2-isobutylphenyl; 2-methoxyphenyl; 2-trifluoromethylphenyl; 3-methylphenyl; 5-isopropyl-2-methylphenyl; 2,6-dichlorophenyl; 3-chloro-4-methoxyphenyl; 5-chloro-2-methoxyphenyl; 5-chloro-2,4-dimethoxyphenyl; 3,5-difluorophenyl; 4-pyridin-3-ylphenyl; 4-cyanophenyl; quinolin-5-yl; quinolin-4-yl; quinolin-2-yl; furan-2-yl; benzofuran-2-yl; 1-difluoromethylbenzimidazol-2-yl; 3,5-dimethylpyrazol-4-yl; tetrahydropyran-3-yl; 3-methylisoxazol-5-yl; tetrahydrofuran-3-yl; 5-fluoropyridin-2-yl; 5-chloropyridin-2-yl; 6-methoxypyridin-3-yl; isoquinolin-3-yl; quinolin-7-yl; 3′-fluorobipheny-3-yl; 4′-methoxybiphen-2-yl; 3-furan-2-ylphenyl; 3′-methoxybiphen-3-yl; 2-propylthiophenyl; 3-thiophen-3-ylphenyl; 3-pyridin-4-ylphenyl; 2-thiophen-2-ylphenyl; 3-pyridin-3-ylphenyl; 3-thiophen-2-ylphenyl; 4′-fluorobiphen-3-yl; 3-(2-methylthiazol-4-yl)phenyl; biphen-2-yl; 2-methoxybiphen-5-yl; 4′-cyanobiphen-3-yl; R-3,3-dimethyltetrahydropyran-4-yl; cyclohexyl; cyclopentyl; phenyl; furan-2-yl; 2-methyl-5-trifluoromethylfuran-4-yl; 5-methyl-1-phenylpyrazol-4-yl; 5-methyl-3-phenylisoxazol-4-yl; 3-methyl-5-phenylisoxazol-4-yl; 2,5-dimethyl-1-phenylpyrazol-4-yl; quinolin-6-yl; 3-methoxyquinolin-6-yl; isoquinolin-6-yl; 3,5-ditrifluoromethylphenyl; 4-ethoxy-5-isopropyl-2-methylphenyl; 4-phenylthiazol-2-yl; 1-methylcyclohexyl; thiophen-3-yl; benzofuran-5-yl; thiophen-5-yl; 5-bromothiophen-2-yl; or 4-bromothiophen-2-yl.

15. The compound of claim 1 wherein Ar is a group of formula (a) wherein A is cycloalkyl optionally substituted as defined.

16. The compound of claim 1 wherein Ar is a group of formula (a) wherein A is monocycle heterocyclyl optionally substituted as defined.

17. The compound of claim 15 wherein Ar¹ is phenyl or heteroaryl each ring optionally substituted with R^{a} which is halo, cyano, or alkoxy.

18. A compound selected from:
(R)-1-(4-(2-methylpyridin-4-yl)phenyl)-3-(1-(naphthalen-2-yl)ethyl)urea;
1-(4-(2-methyl-4-pyridinyl)phenyl)-3-((1S)-1-(2-naphthalenyl)ethyl)urea;
1-((1S)-2-(4-fluorophenyl)-1-methylethyl)-3-(4-(2-methyl-4-pyridinyl)phenyl)urea;
1-((1S)-1-(2-fluorophenyl)ethyl)-3-(4-(2-methyl-4-pyridinyl)phenyl)urea;
1-(4-(2-methyl-4-pyridinyl)phenyl)-3-(2-naphthalenylmethyl)urea;
1-((4R/S)-3,3-dimethyl-3,4-dihydro-2H-chromen-4-yl)-3-(4-(2-methyl-4-pyridinyl)phenyl)urea;
1-(1-(3-fluorophenyl)ethyl)-3-(4-(2-methylpyridin-4-yl)phenyl)urea;
1-(2-(4-bromophenyl)ethyl)-3-(4-(2-methyl-4-pyridinyl)phenyl)urea;
1-((1R)-1-(2-methoxyphenyl)ethyl)-3-(4-(2-methyl-4-pyridinyl)phenyl)urea;
1-(4-(2-methyl-4-pyridinyl)phenyl)-3-(1R)-1-phenylbutyl)urea;
1-((1S)-1-(4-bromophenyl)ethyl)-3-(4-(2-methyl-4-pyridinyl)phenyl)urea;
1-(1-(3-methoxyphenyl)ethyl)-3-(4-(2-methyl-4-pyridinyl)phenyl)urea;
1-cyclopentyl-3-(4-(2-methyl-4-pyridinyl)phenyl)urea;
1-(4-(2-methylpyridin-4-yl)phenyl)-3-(3-morpholinophenyl)urea;
1-(4-(2-methylpyridin-4-yl)phenyl)-3-(1-p-tolylethyl)urea;
1-(1-(4-chlorophenyl)ethyl)-3-(4-(2-methylpyridin-4-yl)phenyl)urea;
1-(4-(2-methylpyridin-4-yl)phenyl)-3-(quinolin-6-yl)urea;
1-(1-(3-chlorophenyl)ethyl)-3-(4-(2-methylpyridin-4-yl)phenyl)urea;
1-(1-(3-bromophenyl)ethyl)-3-(4-(2-methylpyridin-4-yl)phenyl)urea;
1-(1-(2-bromophenyl)ethyl)-3-(4-(2-methylpyridin-4-yl)phenyl)urea;
1-(4-(2-methylpyridin-4-yl)phenyl)-3-(quinolin-4-ylmethyl)urea;
(R)-1-(4-(2-methylpyridin-4-yl)phenyl)-3-(1-(quinolin-2-yl)ethyl)urea;
(S)-1-(4-(2-methylpyridin-4-yl)phenyl)-3-(1-(quinolin-2-yl)ethyl)urea;
(R)-1-(4-(2-methylpyridin-4-yl)phenyl)-3-(1-(quinolin-3-yl)ethyl)urea;
(S)-1-(4-(2-methylpyridin-4-yl)phenyl)-3-(1-(quinolin-3-yl)ethyl)urea;
1-(4-(2-methylpyridin-4-yl)phenyl)-3-(quinolin-2-ylmethyl)urea;
(R)-1-(1-(2-fluorophenyl)ethyl)-3-(4-(2-methylpyridin-4-yl)phenyl)urea;
(S)-1-(1-(2-fluorophenyl)ethyl)-3-(4-(2-methylpyridin-4-yl)phenyl)urea;
1-(5-bromo-2-fluorophenyl)-3-(4-(2-methylpyridin-4-yl)phenyl)urea;
1-(1-(furan-2-yl)ethyl)-3-(4-(2-methylpyridin-4-yl)phenyl)urea;
1-(1-(benzofuran-2-yl)ethyl)-3-(4-(2-methylpyridin-4-yl)phenyl)urea;
1-(1-(1-(difluoromethyl)-1H-benzo[d]imidazol-2-yl)ethyl)-3-(4-(2-methylpyridin-4-yl)phenyl)urea;
1-(1-(1-ethyl-3,5-dimethyl-1H-pyrazol-4-yl)ethyl)-3-(4-(2-methylpyridin-4-yl)phenyl)urea;
1-(4-(2-methylpyridin-4-yl)phenyl)-3-(tetrahydro-2H-pyran-3-yl)urea;
1-((3-methylisoxazol-5-yl)methyl)-3-(4-(2-methylpyridin-4-yl)phenyl)urea;
1-cyclohexyl-3-(4-(2-methyl-4-pyridinyl)phenyl)urea;
(R)-1-(3,3-dimethyltetrahydro-2H-pyran-4-yl)-3-(4-(2-methylpyridin-4-yl)phenyl)urea;
1-((1-(2-methoxyphenyl)cyclobutyl)methyl)-3-(4-(2-methylpyridin-4-yl)phenyl)urea;
1-(4-(2-methylpyridin-4-yl)phenyl)-3-(tetrahydrofuran-3-yl)urea;
1-(2-methyl-5-(1-methylethyl)phenyl)-3-(4-(2-methyl-4-pyridinyl)phenyl)urea;
1-(4-(2-methylpyridin-4-yl)phenyl)-3-(2,2,2-trifluoro-1-phenylethyl)urea;
1-(1-(2-chlorophenyl)ethyl)-3-(4-(2-methylpyridin-4-yl)phenyl)urea;
1-(4-(2-methylpyridin-4-yl)phenyl)-3-(1-o-tolylethyl)urea;
1-(1-(5-fluoropyridin-2-yl)ethyl)-3-(4-(2-methylpyridin-4-yl)phenyl)urea;
1-(1-(5-chloropyridin-2-yl)ethyl)-3-(4-(2-methylpyridin-4-yl)phenyl)urea;
1-(1-(6-methoxypyridin-3-yl)ethyl)-3-(4-(2-methylpyridin-4-yl)phenyl)urea;
1-(3-fluorophenyl)-3-(4-(2-methyl-4-pyridinyl)phenyl)urea;
1-(1-(3-(difluoromethoxy)phenyl)ethyl)-3-(4-(2-methylpyridin-4-yl)phenyl)urea;
1-(1-(isoquinolin-3-yl)ethyl)-3-(4-(2-methylpyridin-4-yl)phenyl)urea;
1-(4-(2-methylpyridin-4-yl)phenyl)-3-(1-(quinolin-7-yl)ethyl)urea;
1-(2-fluoro-1-phenylethyl)-3-(4-(2-methylpyridin-4-yl)phenyl)urea;
1-(2-methoxy-1-phenyl ethyl)-3-(4-(2-methylpyridin-4-yl)phenyl)urea;
1-(2-fluoro-2-methyl-1-phenylpropyl)-3-(4-(2-methylpyridin-4-yl)phenyl)urea;
1-(1-(2,4-dichlorophenyl)ethyl)-3-(4-(2-methylpyridin-4-yl)phenyl)urea;
1-(1-(4-methoxyphenyl)ethyl)-3-(4-(2-methylpyridin-4-yl)phenyl)urea;
(S)-1-(3-methyl-2-oxo-2,3,4,5-tetrahydro-1H-benzo[d]azepin-1-yl)-3-(4-(2-methylpyridin-4-yl)phenyl)urea;
1-(2-bromophenyl)-3-(4-(2-methyl-4-pyridinyl)phenyl)urea;
1-(3′-fluoro-3-biphenylyl)-3-(4-(2-methyl-4-pyridinyl)phenyl)urea;
1-(4′-methoxy-2-biphenylyl)-3-(4-(2-methyl-4-pyridinyl)phenyl)urea;
1-(3-(2-furanyl)phenyl)-3-(4-(2-methyl-4-pyridinyl)phenyl)urea;
1-(3-butoxyphenyl)-3-(4-(2-methyl-4-pyridinyl)phenyl)urea;
1-(3-methylphenyl)-3-(4-(2-methyl-4-pyridinyl)phenyl)urea;
1-(3′-methoxy-3-biphenylyl)-3-(4-(2-methyl-4-pyridinyl)phenyl)urea;
1-(4-(2-methyl-4-pyridinyl)phenyl)-3-(2-(propylsulfanyl)phenyl)urea;
1-(4-(2-methyl-4-pyridinyl)phenyl)-3-(3-(3-thiophenyl)phenyl)urea;
1-(4-(2-methyl-4-pyridinyl)phenyl)-3-(3-(4-pyridinyl)phenyl)urea;
1-(5-cyano-2-fluorophenyl)-3-(4-(2-methyl-4-pyridinyl)phenyl)urea;
1-(5-cyano-2-methylphenyl)-3-(4-(2-methyl-4-pyridinyl)phenyl)urea;
1-(4-(2-methyl-4-pyridinyl)phenyl)-3-(2-(2-thiophenyl)phenyl)urea;
1-(4-(2-methyl-4-pyridinyl)phenyl)-3-(3-(3-pyridinyl)phenyl)urea;
1-(4-(2-methyl-4-pyridinyl)phenyl)-3-(3-(trifluoromethyl)phenyl)urea;
1-(5-chloro-2-fluorophenyl)-3-(4-(2-methyl-4-pyridinyl)phenyl)urea;
1-(2-(1-methylpropyl)phenyl)-3-(4-(2-methyl-4-pyridinyl)phenyl)urea;
1-(2-methoxyphenyl)-3-(4-(2-methyl-4-pyridinyl)phenyl)urea;
1-(3-(methoxymethyl)phenyl)-3-(4-(2-methyl-4-pyridinyl)phenyl)urea;
1-(4-(2-methyl-4-pyridinyl)phenyl)-3-(3-(2-thiophenyl)phenyl)urea;
1-(4-(2-methyl-4-pyridinyl)phenyl)-3-(2-methyl-5-(trifluoromethyl)phenyl)urea;
1-(4-(2-methyl-4-pyridinyl)phenyl)-3-(3-propoxyphenyl)urea;
1-(4-(2-methyl-4-pyridinyl)phenyl)-3-(2-propoxyphenyl)urea;
1-(5-tert-butyl-2-methoxyphenyl)-3-(4-(2-methyl-4-pyridinyl)phenyl)urea;
1-(3-tert-butylphenyl)-3-(4-(2-methyl-4-pyridinyl)phenyl)urea;
1-(2-(benzyloxy)phenyl)-3-(4-(2-methyl-4-pyridinyl)phenyl)urea;
1-(4-(2-methyl-4-pyridinyl)phenyl)-3-(2-(3-pyridinyl)phenyl)urea;
1-(2-(1-methylethyl)phenyl)-3-(4-(2-methyl-4-pyridinyl)phenyl)urea;
1-(5-bromo-2-methylphenyl)-3-(4-(2-methyl-4-pyridinyl)phenyl)urea;
1-(4-(2-methyl-4-pyridinyl)phenyl)-3-(2-propylphenyl)urea;
1-(5-chloro-2-methylphenyl)-3-(4-(2-methyl-4-pyridinyl)phenyl)urea;
1-(3-ethoxyphenyl)-3-(4-(2-methyl-4-pyridinyl)phenyl)urea;
1-(2-fluoro-5-(trifluoromethyl)phenyl)-3-(4-(2-methyl-4-pyridinyl)phenyl)urea;
1-(2-ethylphenyl)-3-(4-(2-methyl-4-pyridinyl)phenyl)urea;
1-(4-(2-methyl-4-pyridinyl)phenyl)-3-(3-(methylsulfanyl)phenyl)urea;
1-(2,5-difluorophenyl)-3-(4-(2-methyl-4-pyridinyl)phenyl)urea;
1-(5-chloro-2-cyanophenyl)-3-(4-(2-methyl-4-pyridinyl)phenyl)urea;
1-(2-ethoxyphenyl)-3-(4-(2-methyl-4-pyridinyl)phenyl)urea;
1-(4′-fluoro-3-biphenylyl)-3-(4-(2-methyl-4-pyridinyl)phenyl)urea;
1-(5-fluoro-2-methoxyphenyl)-3-(4-(2-methyl-4-pyridinyl)phenyl)urea;
1-(2-methoxy-5-(trifluoromethyl)phenyl)-3-(4-(2-methyl-4-pyridinyl)phenyl)urea;
1-(4-(2-methyl-4-pyridinyl)phenyl)-3-(3-(2-methyl-1,3-thiazol-4-yl)phenyl)urea;
1-(2-biphenylyl)-3-(4-(2-methyl-4-pyridinyl)phenyl)urea;
1-(2-cyanophenyl)-3-(4-(2-methyl-4-pyridinyl)phenyl)urea;
1-(4-(2-methyl-4-pyridinyl)phenyl)-3-(2-(phenylcarbonyl)phenyl)urea;
1-(4-(2-methyl-4-pyridinyl)phenyl)-3-(2-(trifluoromethyl)phenyl)urea;
1-(5-fluoro-2-methylphenyl)-3-(4-(2-methyl-4-pyridinyl)phenyl)urea;
1-(2-chloro-5-methoxyphenyl)-3-(4-(2-methyl-4-pyridinyl)phenyl)urea;
1-(2-chloro-5-methylphenyl)-3-(4-(2-methyl-4-pyridinyl)phenyl)urea;
1-(4-(2-methyl-4-pyridinyl)phenyl)-3-(2-phenoxyphenyl)urea;
1-(3-cyanophenyl)-3-(4-(2-methyl-4-pyridinyl)phenyl)urea;
1-(3-acetylphenyl)-3-(4-(2-methyl-4-pyridinyl)phenyl)urea;
1-1-(4-methoxybiphenyl-3-yl)-3-(4-(2-methylpyridin-4-yl)phenyl)urea;
1-(2-cyano-5-methylphenyl)-3-(4-(2-methyl-4-pyridinyl)phenyl)urea;
1-(3-(1-methylethoxy)phenyl)-3-(4-(2-methyl-4-pyridinyl)phenyl)urea;
1-(4-(2-methyl-4-pyridinyl)phenyl)-3-(2-(methylsulfanyl)phenyl)urea;
1-(4-(2-methyl-4-pyridinyl)phenyl)-3-(3-(phenylcarbonyl)phenyl)urea;
1-(2-chloro-5-cyanophenyl)-3-(4-(2-methyl-4-pyridinyl)phenyl)urea;
1-(4′-cyano-3-biphenylyl)-3-(4-(2-methyl-4-pyridinyl)phenyl)urea;
1-(2-fluoro-5-methylphenyl)-3-(4-(2-methyl-4-pyridinyl)phenyl)urea;
1-(4-(2-methyl-4-pyridinyl)phenyl)-3-(3-phenoxyphenyl)urea;
1-(2-methoxy-5-methylphenyl)-3-(4-(2-methyl-4-pyridinyl)phenyl)urea;
1-(2-chloro-5-(trifluoromethyl)phenyl)-3-(4-(2-methyl-4-pyridinyl)phenyl)urea;
1-(2,5-dichlorophenyl)-3-(4-(2-methyl-4-pyridinyl)phenyl)urea;
1-(2-fluorophenyl)-3-(4-(2-methyl-4-pyridinyl)phenyl)urea;
1-(3-methoxyphenyl)-3-(4-(2-methyl-4-pyridinyl)phenyl)urea;
1-(4-(2-methylpyridin-4-yl)phenyl)-3-((1R,2S)-2-phenylcyclopropyl)urea;
1-(4-(2-methyl-4-pyridinyl)phenyl)-3-((1R)-1-phenylpropyl)urea;
1-(4-(2-methyl-4-pyridinyl)phenyl)-3-((1S)-1-phenylpropyl)urea;
1-(2-(4-chlorophenyl)ethyl)-3-(4-(2-methyl-4-pyridinyl)phenyl)urea;
1-(4-(2-methylpyridin-4-yl)phenyl)-3-phenyl)urea;
1-(furan-2-ylmethyl)-3-(4-(2-methylpyridin-4-yl)phenyl)urea;
1-(5-methyl-2-(trifluoromethyl)furan-3-yl)-3-(4-(2-methylpyridin-4-yl)phenyl)urea;
1-(5-methyl-1-phenyl-1H-pyrazol-4-yl)-3-(4-(2-methylpyridin-4-yl)phenyl)urea;
1-(5-methyl-3-phenylisoxazol-4-yl)-3-(4-(2-methylpyridin-4-yl)phenyl)urea;
1-(3-methyl-5-phenylisoxazol-4-yl)-3-(4-(2-methylpyridin-4-yl)phenyl)urea;
1-(3,5-dimethyl-1-phenyl-1H-pyrazol-4-yl)-3-(4-(2-methylpyridin-4-yl)phenyl)urea;
1-(2,6-dichlorophenyl)-3-(4-(2-methylpyridin-4-yl)phenyl)urea;
1-(3-chloro-4-methoxyphenyl)-3-(4-(2-methyl-4-pyridinyl)phenyl)urea;
1-(5-chloro-2-methoxyphenyl)-3-(4-(2-methyl-4-pyridinyl)phenyl)urea;
1-(5-chloro-2,4-dimethoxyphenyl)-3-(4-(2-methyl-4-pyridinyl)phenyl)urea;
1-(3,5-difluorophenyl)-3-(4-(2-methyl-4-pyridinyl)phenyl)urea;
1-(4-cyanophenethyl)-3-(4-(2-methylpyridin-4-yl)phenyl)urea;
1-(1-(2-bromophenyl)-3-methylbutyl)-3-(4-(2-methylpyridin-4-yl)phenyl)urea;
1-(4-(2-methylpyridin-4-yl)phenyl)-3-(1-(4-(pyridin-3-yl)phenyl)ethyl)urea;
1-(1-(3-cyanophenyl)ethyl)-3-(4-(2-methylpyridin-4-yl)phenyl)urea;
1-(1-(4-cyanophenyl)ethyl)-3-(4-(2-methylpyridin-4-yl)phenyl)urea;
1-(4-(2-methylpyridin-4-yl)phenyl)-3-(1-m-tolylethyl)urea;
1-(4-(2-methylpyridin-4-yl)phenyl)-3-(1-(quinolin-6-yl)ethyl)urea;
1-(1-(3-methoxyquinolin-6-yl)ethyl)-3-(4-(2-methylpyridin-4-yl)phenyl)urea;
1-(3-methyl-1-phenylbutyl)-3-(4-(2-methylpyridin-4-yl)phenyl)urea;
1-(2,2-difluoro-1-phenyl)ethyl)-3-(4-(2-methylpyridin-4-yl)phenyl)urea;
1-(1-(isoquinolin-6-yl)ethyl)-3-(4-(2-methylpyridin-4-yl)phenyl)urea;
1-(1-(3,5-bis(trifluoromethyl)phenyl)ethyl)-3-(4-(2-methylpyridin-4-yl)phenyl)urea;
1-(4-ethoxy-5-isopropyl-2-methylphenyl)-3-(4-(2-methylpyridin-4-yl)phenyl)urea;
1-(4-(2-methylpyridin-4-yl)phenyl)-3-(4-phenylthiazol-2-yl)urea;
1-(4-(2-methylpyridin-4-yl)phenyl)-3-(1-phenylcyclopropyl)urea;
1-(4-(2-cethylpyridin-4-yl)phenyl)-3-(1-phenylcyclobutyl)urea;
1-(4-(2-methylpyridin-4-yl)phenyl)-3-((1-phenylcyclopentyl)methyl)urea;
1-(4-(2-methylpyridin-4-yl)phenyl)-3-((1-phenylcyclobutyl)methyl)urea;
1-(2-methyl-2-phenylpropyl)-3-(4-(2-methylpyridin-4-yl)phenyl)urea;
1-(4-(2-methylpyridin-4-yl)phenyl)-3-(4-phenyltetrahydro-2H-pyran-4-yl)methyl)urea;
1-(4-(2-methylpyridin-4-yl)phenyl)-3-((1-phenylcyclopropyl)methyl)urea;
1-(3-(4-chlorophenyl)tetrahydrofuran-3-yl)-3-(4-(2-methylpyridin-4-yl)phenyl)urea;
1-(1-(4-bromophenyl)cyclobutyl)-3-(4-(2-methylpyridin-4-yl)phenyl)urea;
1-(2-(2-fluorophenyl) propan-2-yl)-3-(4-(2-methylpyridin-4-yl)phenyl)urea;
1-(1-(4-fluorophenyl)cyclohexyl)-3-(4-(2-methylpyridin-4-yl)phenyl)urea;
1-(1-(3-fluorophenyl)cyclohexyl)-3-(4-(2-methylpyridin-4-yl)phenyl)urea;
1-(1-(3-fluorophenyl)cyclohexyl)-3-(3-methoxy-4-(2-methylpyridin-4-yl)phenyl)urea;
1-(1-(2-fluorophenyl)cyclohexyl)-3-(4-(2-methylpyridin-4-yl)phenyl)urea;
1-((1-(6-chloropyridin-2-yl) cyclopentyl)methyl)-3-(4-(2-methylpyridin-4-yl)phenyl)urea;
1-(4-(2-methylpyridin-4-yl)phenyl)-3-((1-(pyridin-2-yl)cyclopentyl)methyl)urea;
1-(1-(3-bromophenyl)cyclobutyl)-3-(4-(2-methylpyridin-4-yl)phenyl)urea;
1-((1-(2-chloropyridin-4-yl)cyclopentyl)methyl)-3-(4-(2-methylpyridin-4-yl)phenyl)urea;
1-(4-(2-methyl pyridin-4-yl)phenyl)-3-4′-(pyridin-4-yl)cyclopentyl)methyl)urea;
1-((1-(2-cyanopyridin-4-yl)cyclopentyl)-methyl)-3-(4-(2-methylpyridin-4-yl)phenyl)urea;
1-(1-(4-cyanophenyl)cyclobutyl)-3-(4-(2-methylpyridin-4-yl)phenyl)urea;
1-(1-(6-chloropyridin-2-yl)cyclopentyl)-3-(4-(2-methylpyridin-4-yl)phenyl)urea1-(4-(2-methylpyridin-4-yl)phenyl)-3-(1-(pyridin-2-yl)cyclopentyl)urea;
1-(1-(2-chloropyridin-4-yl)cyclopentyl)-3-(4-(2-methylpyridin-4-yl)phenyl)urea;
1-(4-(2-methylpyridin-4-yl)phenyl)-3-(1-(pyridin-4-yl)cyclopentyl)urea;
1-(4-(2-methylpyridin-4-yl)phenyl)-3-(1-(pyridin-4-yl)cyclopentyl)urea;
1-(1-(6-fluoropyridin-2-yl)cyclopentyl)-3-(4-(2-methylpyridin-4-yl)phenyl)urea;
1-(1-(6-chloropyridin-2-yl)cyclobutyl)-3-(4-(2-methylpyridin-4-yl)phenyl)urea;
1-(4-(2-methylpyridin-4-yl)phenyl)-3-(1-(pyridin-3-yl)cyclopentyl)urea;
1-(4-(2-methylpyridin-4-yl)phenyl)-3-(2-phenylbutan-2-yl)urea;
1-(1-(6-methoxypyridin-2-yl)cyclopentyl)-3-(4-(2-methylpyridin-4-yl)phenyl)urea;
1-(4-hydroxy-1-phenylcyclohexyl)-3-(4-(2-methylpyridin-4-yl)phenyl)urea;
1-(4,4-difluoro-1-phenylcyclohexyl)-3-(4-(2-methylpyridin-4-yl)phenyl)urea;
1-(2-(3-fluorophenyl)propan-2-yl)-3-(4-(2-methylpyridin-4-yl)phenyl)urea;
1-(4-(2-methylpyridin-4-yl)phenyl)-3-(2-(thiophen-3-yl)propan-2-yl)urea;
1-(2-(benzofuran-5-yl)propan-2-yl)-3-(4-(2-methylpyridin-4-yl)phenyl)urea;
1-(2-(benzo thiophen-5-yl)propan-2-yl)-3-(4-(2-methylpyridin-4-yl)phenyl)urea;
1-(2-(5-bromothiophen-2-yl)propan-2-yl)-3-(4-(2-methylpyridin-4-yl)phenyl)urea;
1-(2-(4-bromothiophen-2-yl)propan-2-yl)-3-(4-(2-methylpyridin-4-yl)phenyl)urea; or
1-((1-(2-methoxyphenyl)cyclobutyl)methyl)-3-(4-(2-methylpyridin-4-yl)phenyl)urea; or a pharmaceutically acceptable salt thereof.

19. A pharmaceutical composition comprising a compound of any of the claims 1-18 and a pharmaceutically acceptable excipient or a mixture of a compound of any of the claims 1-18 and a pharmaceutically acceptable salt thereof and a pharmaceutically acceptable excipient.

20. A method of treating Alzheimer's disease by modulation of γ-secretase in a patient which method comprises administering to the patient a pharmaceutical composition comprising a compound of any of the claims 1-18 and a pharmaceutically acceptable excipient or a mixture of a compound of any of the claims 1-18 and a pharmaceutically acceptable salt thereof and a pharmaceutically acceptable excipient.

21. (canceled)
